(19) United States
(12) Patent Application Publication
(10) Pub. No.: US 2012/0077737 A1
(43) Pub. Date: Mar. 29, 2012

(54) HEPATITIS C INHIBITOR COMPOUNDS

(75) Inventors: Montse Llinas-Brunet, Dollard-des-Ormeaux (CA); Josée Bordeleau, Laval (CA); Cédrickx Godbout, Mascouche (CA); Mélissa Leblanc, Laval (CA); Benoît Moreau, Laval (CA); Jeffrey O'Meara, Boisbriand (CA)

(73) Assignee: BOEHRINGER INGELHEIM INTERNATIONAL GMBH, Ingelheim am Rhein (DE)

(21) Appl. No.: 12/952,777

(22) Filed: Nov. 23, 2010

Related U.S. Application Data
(60) Provisional application No. 61/264,067, filed on Nov. 24, 2009.

(51) Int. Cl. A61K 38/07; A61P 31/14; C07K 5/117

(52) U.S. Cl. 514/4.3; 530/330

(57) ABSTRACT

Compounds of the invention, which are macrocyclic peptide analogs containing an acylsulfonamide moiety, maintain good activity against NS3 proteases containing clinically relevant resistance mutations for this class as represented by genotype 1a R155K, genotype 1b D168V and genotype 1a D168V resistance mutations. The compounds of the invention are useful as inhibitors of HCV NS3 protease for the treatment of hepatitis C viral infection.

### FIELD OF THE INVENTION

The present invention relates to macrocycle peptide analogs and their use as inhibitors of hepatitis C virus (HCV) NS3 protease activity, pharmaceutical composition containing the same, and methods of using the same for the treatment of HCV infection.

### BACKGROUND OF THE INVENTION

It is estimated that at least 170 million persons worldwide are infected with the hepatitis C virus (HCV). Acute HCV infection progresses to chronic infection in a high number of cases, and, in some infected individuals, chronic infection leads to serious liver diseases such as cirrhosis and hepatocellular carcinoma.

HCV replicates to very high levels and the HCV polymerase is error-prone resulting in a wide variety of new sequence variants (Science 1998, 282, 103-107). Some new sequence variants confer resistance to drug candidates currently undergoing clinical trials. The emergence of such resistance mutations is one cause of treatment failure in HCV antiviral trials (New England Journal of Medicine 2009, 360, 1827-1838 and New England Journal of Medicine 2009, 360, 1839-1850). Resistance mutations observed in the clinical trials can also be selected for by in vitro experiments, with correlation between clinical resistance mutations and those from in vitro experiments (New England Journal of Medicine 2009, 360, 1827-1838).

The impact of emergence of any one resistance mutant on the outcome of therapy is determined not only by the effect of the particular resistance mutant on drug potency, but also by the fitness of the resulting virus variant. A resistance mutation which results in a virus with poor fitness will be more difficult to select under drug pressure, even if it results in a large decrease in potency for the drug. As a result not all resistance mutations observed are equally relevant to clinical therapy. (Antimicrobial Agents for Chemotherapy 2008, 52, 1101-1110)

There is a need for new antivirals with activity against known, relatively fit, resistance mutations for their target. In addition, a patient who has previously failed treatment with a HCV drug of a particular class (e.g. an HCV protease inhibitor) may be treated with another drug of that same class (e.g. another HCV protease inhibitor) if activity of the second drug is not affected by the resistance mutations selected on the earlier treatment. This has been demonstrated for HIV antiviral therapies (Journal of Medical Virology 2008, 80, 565-576).

HCV NS3 protease inhibitors currently in the clinic primarily target HCV genotype 1 infection. The vast majority of HCV genotype 1 infections are of either subtype 1a or subtype 1b (Clinics in Liver Disease 2003, 7, 45-66). The NS3 proteases from HCV-1a and HCV-1b subtypes have very similar but not identical sequences. HCV protease inhibitors currently in clinical trials can be divided into two classes based on their chemical structure, and these classes have distinct but overlapping resistance mutation profiles (Journal of Viral Hepatitis 2009, 16, 377-387). A first class, as exemplified by the inhibitors telaprevir and boceprevir, contain an α-ketoamide moiety as the active site binding group; characteristic mutations for these compounds result in substitutions at amino acids 36, 41, 54, 155, 156, and 170 of the NS3 protease. Compounds containing an acylsulfonamide in place of the α-ketoamide, for example ITMN-191, TMC435, and MK-7009, are recognized as being part of a second class.

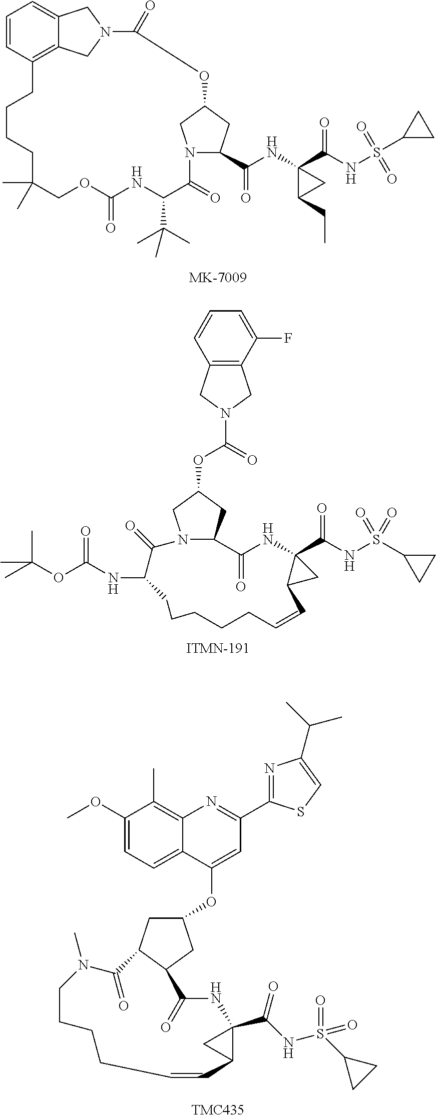

Resistance against this second class of protease inhibitors is primarily due to substitutions at amino acid 155, typically Arg to Lys (R155K), at amino acid 156, typically Ala to Val (A156V) or Ala to Thr (A156T), and at amino acid 168, typically Asp to Val (D168V) or Asp to Ala (D168A). Arg-155 and Ala-156 substitutions are observed for both classes (Antimicrobial Agents and Chemotherapy 2009, 53 (4) 1377-85, Antimicrobial Agents and Chemotherapy 2008, 52 (12) 4432-41, Antimicrobial Agents and Chemotherapy 2009, published online 19 Oct. 2009 doi:10.1128/AAC.00677-09, Journal of Viral Hepatitis 2009, 16, 377-387). There is increasing evidence that virus containing the R155K mutation is quite fit and can persist for long periods of time (Journal of Infectious Diseases 2009, 199:737-41). Virus containing the A156V or T mutations is not very fit and even though these are observed in the clinic they are transient and revert to wildtype in a short period of time. Virus containing the D168A mutation has relatively poor fitness. D168V appears to have intermediate fitness, though there is limited clinical data on the persistence of this variant in patients. (Expert Opinion on Investigational Drugs 2008 17(3):303-319, Antimicrobial Agents for Chemotherapy 2008, 52, 1101-1110, Hepatology 2007, 46, 631-639, Gastroenterology, 2007, 132, 1767-1777)

R155K has relatively greater fitness, whereas A156TN and D168A have relatively poor fitness. (He et al. Antimicrobial Agents for Chemotherapy, 2008, 52, 1101-1110, Kieffer et al. Hepatology, 2007, 46, 631-639, Sarrazin et al. Gastroenterology, 2007, 132, 1767-1777). D168V appears to have intermediate fitness, though there is limited clinical data (He et al. Antimicrobial Agents for Chemotherapy, 2008, 52, 1101-1110).

An R155K substitution appears in genotype 1a patients as the R155K mutation results from a single-base mutation but more rarely in genotype 1b patients which require a two-base mutation for the same substitution to occur. D168V can occur via a single-base mutation in either subtype 1a or 1b, but in clinical trials disclosed to date, it occurs more commonly in genotype 1b patients, (Marcellin et al., Antiviral activity and safety of TMC435 combined with peginterferon alpha-2A and ribavirin in patients with genotype 1 hepatitis C infection who failed previous IFN-based therapy, 44th Annual Meeting of the European Association for the Study of the Liver, Apr. 22-26, 2009, Copenhagen, Denmark, Lenz et al., In vitro resistance profile of the HCV NS3/4A inhibitor TMC435350, 15^{th} International Symposium on Hepatitis C Virus & Related Viruses, San Antonio, Tex., USA, Oct. 5-9, 2008) probably because for genotype 1a patients the more fit R155K is typically observed instead.

Even though in genotype 1a infections R155K is more fit than D168V, there is a risk that resistance due to D168V could occur in these patients, so it is preferred to identify inhibitors which are also active against genotype 1a D168V. Accordingly, clinically relevant resistance mutations for the second class of HCV protease inhibitors are considered genotype 1a R155K, genotype 1b D168V, and genotype 1a D168V.

Activity of HCV protease inhibitors is most effectively measured using the subgenomic replicon system, in which inhibition of the physiologically relevant HCV replication complex can be directly measured (Journal of Viral Hepatitis, 2007, 14 (Suppl. 1) 64-67). Inhibition in this system has translated into clinical efficacy as shown for all the clinical candidates described above (Antimicrobial Agents and Chemotherapy 2009, 53(4) 1377-85, Antimicrobial Agents and Chemotherapy 2008, 52(12) 4432-41, Antimicrobial Agents and Chemotherapy 2009, published online 19 Oct. 2009 doi:10.1128/AAC.00677-09).

Accordingly, there is a need to provide novel compounds as drug candidates that are active against clinically relevant resistance mutations as represented by genotype 1a R155K, genotype 1b D168V and genotype 1a D168V.

WO 2007/056120 describes macrocyclic peptides that are useful for inhibiting HCV.

### SUMMARY OF THE INVENTION

We have unexpectedly found that compounds of the invention maintain good activity against NS3 proteases containing clinically relevant resistance mutations for this class as represented by genotype 1a R155K, genotype 1b D168V and genotype 1a D168V resistance mutations.

Further objects of this invention arise for the one skilled in the art from the following description and the examples.

One aspect of the invention provides a compound or a salt thereof according to the following structures:

| Compound # | Structure |
|---|---|
| 1001 | 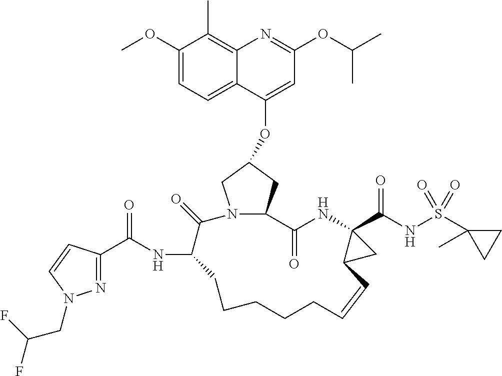 |
| 1002 | 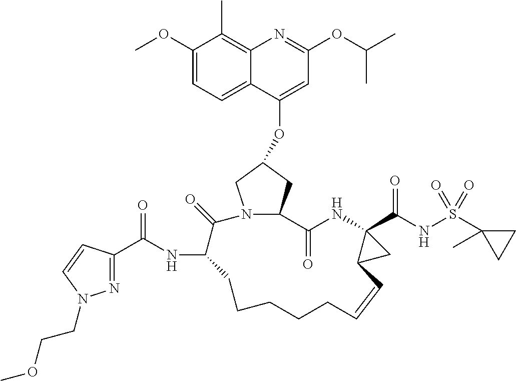 |
| 1003 | 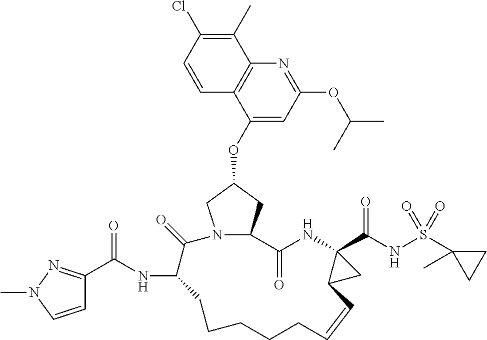 |
| 1004 | 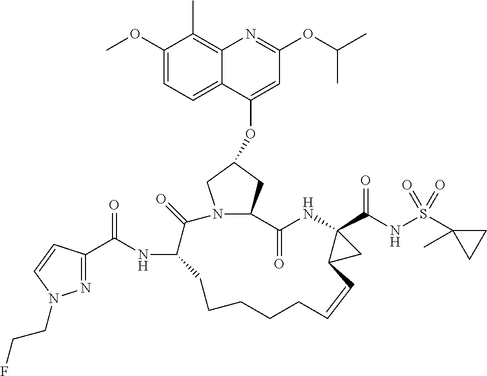 |
| 1005 | 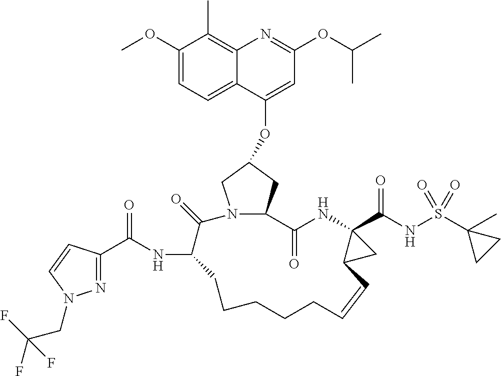 |
| 1006 | 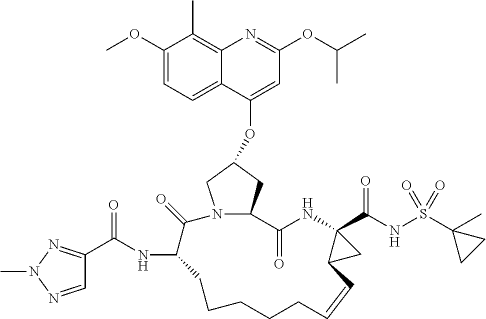 |
| 1007 | 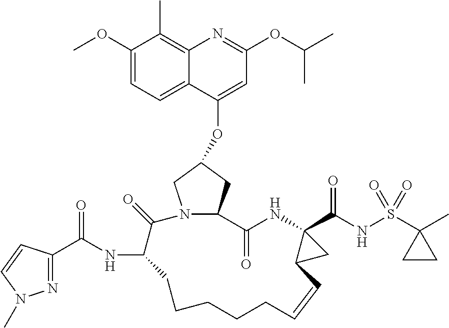 |
| 1008 | 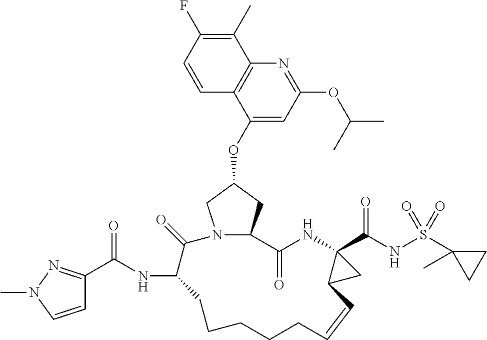 |
| 1009 | 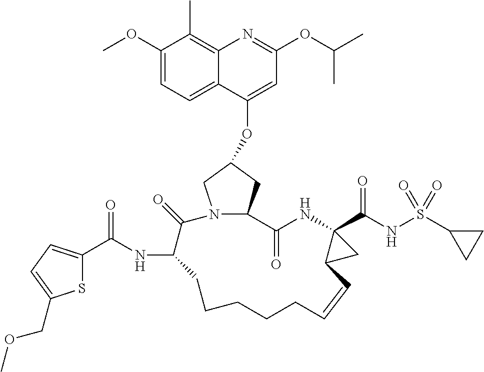 |
| 1010 | 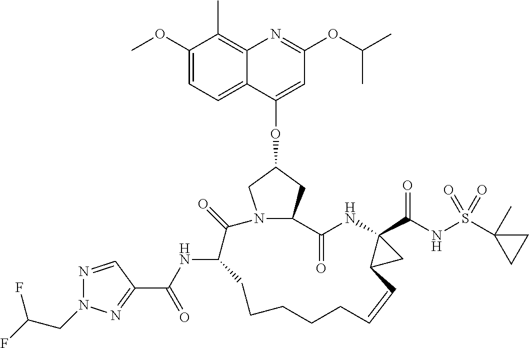 |
| 1011 | 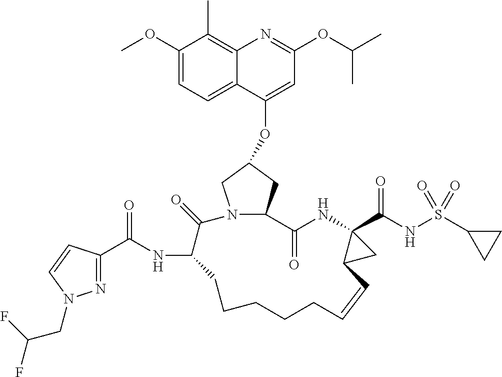 |
| 1012 | 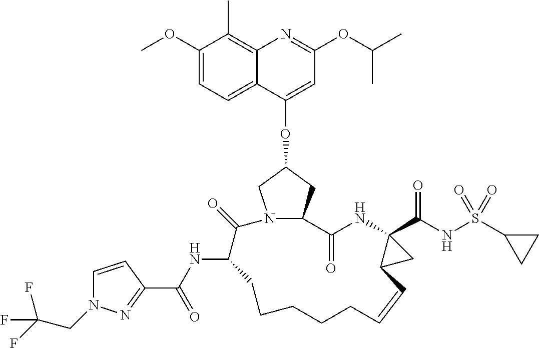 |
| 1013 | 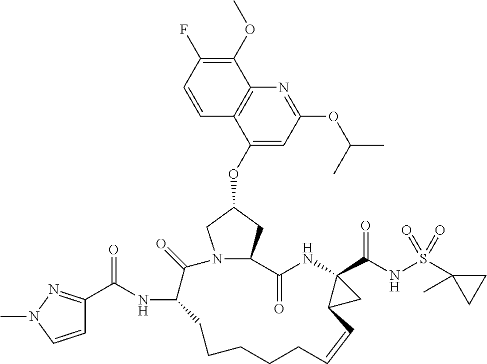 |
| 1014 | 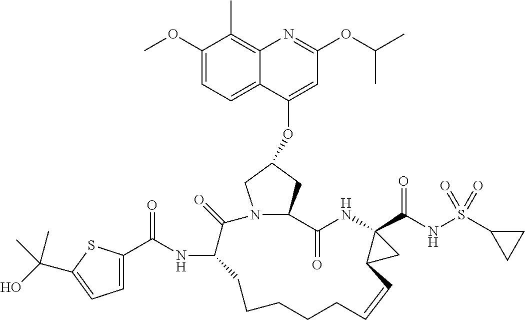 |
| 1015 | 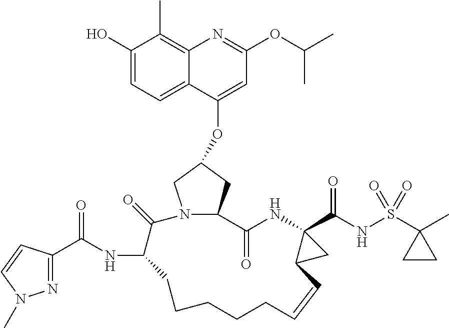 |
| 1016 | 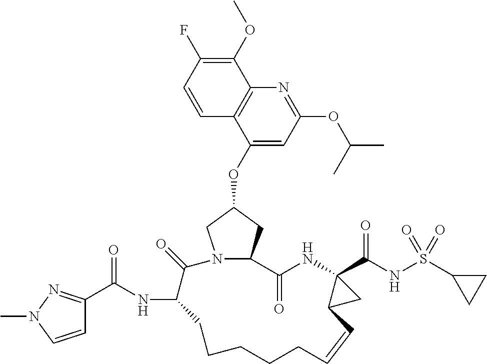 |
| 1017 | 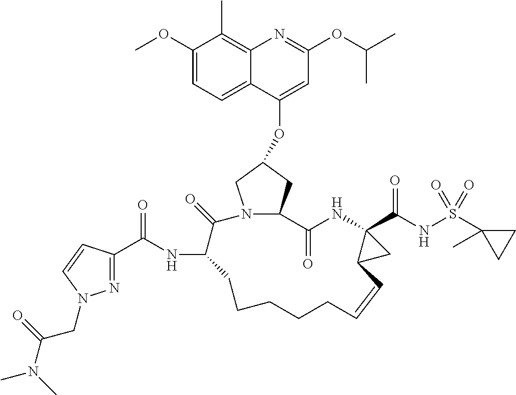 |
| 1018 | 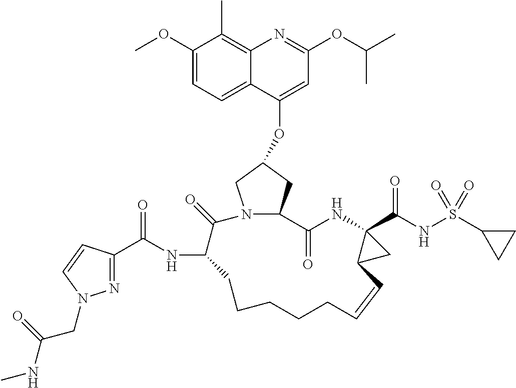 |
| 1019 | 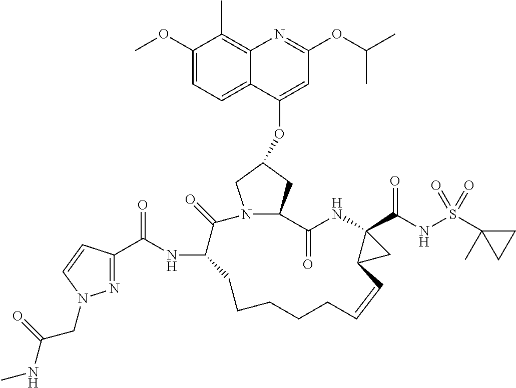 |
| 1020 | 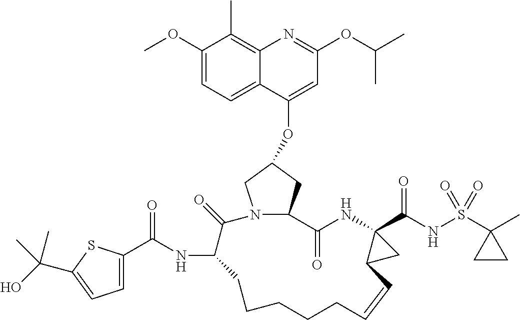 |
| 1021 | 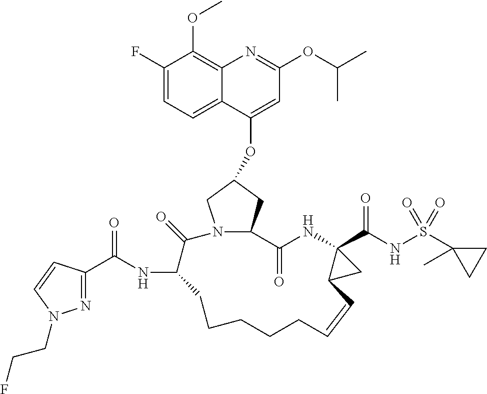 |
| 1022 | 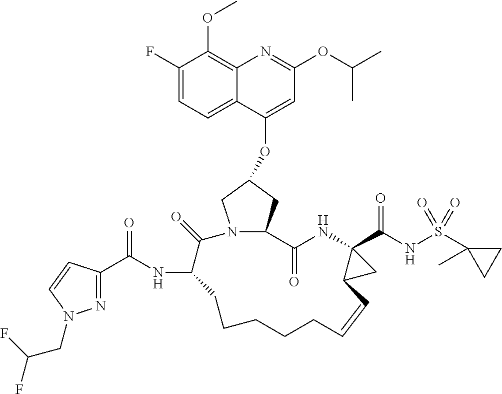 |
| 1023 | 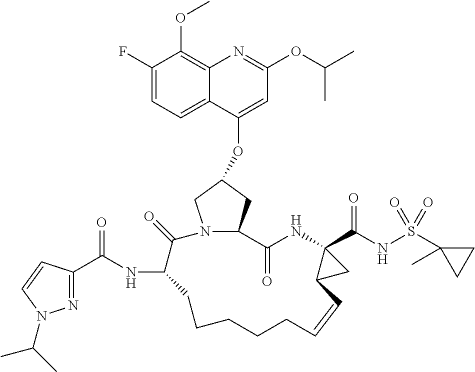 |
| 1024 | 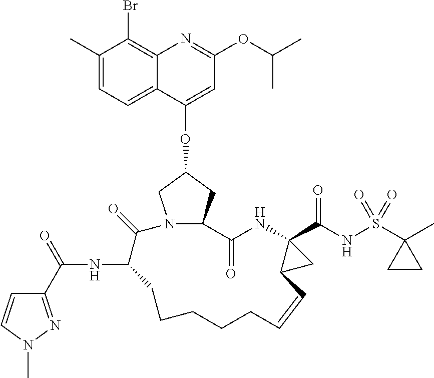 |
| 1025 | 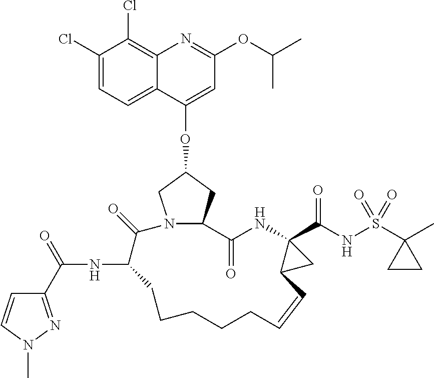 |
| 1026 | 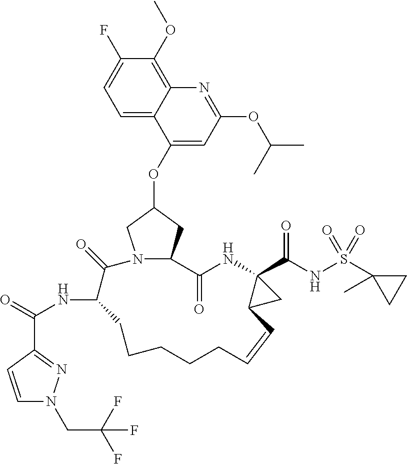 |
| 1027 | 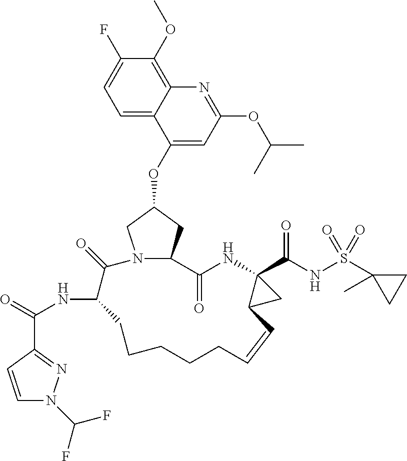 |
| 1028 | 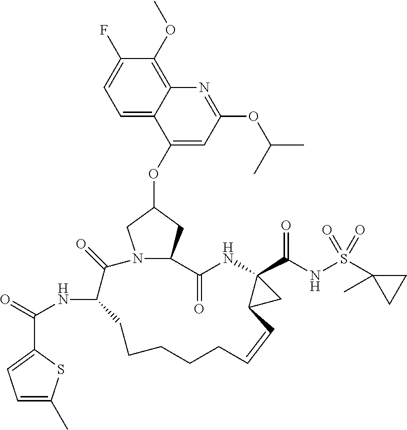 |
| 1029 | 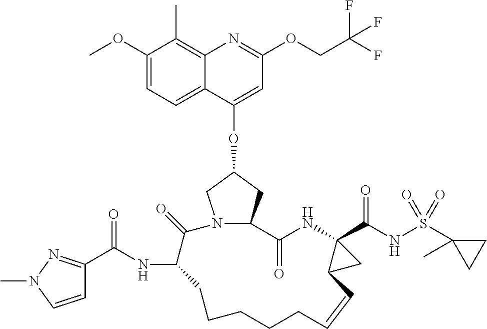 |

Another aspect of this invention provides compounds 1001-1029 that exhibit unexpectedly good cell-based potency against genotype 1a R155K, genotype 1b D168V and genotype 1a D168V resistance mutations.

Another aspect of this invention provides any one of compounds 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029 and their pharmaceutically acceptable salts forms that exhibit unexpectedly good cell-based potency against genotype 1a R155K, genotype 1b D168V and genotype 1a D168V resistance mutations.

Another aspect of this invention provides compounds of the invention, or a pharmaceutically acceptable salt thereof, as a medicament.

Included within the scope of this invention is a pharmaceutical composition comprising an anti-hepatitis C virally effective amount of a compound of the invention, or a pharmaceutically acceptable salt thereof, in admixture with at least one pharmaceutically acceptable carrier medium or auxiliary agent.

According to a further aspect of this embodiment the pharmaceutical composition according to this invention further comprises a therapeutically effective amount of at least one other antiviral agent.

The invention also provides the use of a pharmaceutical composition as described hereinabove for the treatment of a hepatitis C viral infection in a human being having or at risk of having the infection.

Another important aspect of the invention involves a method of treating or preventing a hepatitis C viral infection in a human being by administering to the human being an anti-hepatitis C virally effective amount of a compound of the invention, a pharmaceutically acceptable salt thereof, or a composition as described above, alone or in combination with at least one other antiviral agent, administered together or separately.

Also within the scope of this invention is the use of a compound of the invention, or a pharmaceutically acceptable salt thereof, as described herein, for the manufacture of a medicament for the treatment or prevention of hepatitis C viral infection in a human being.

An additional aspect of this invention refers to an article of manufacture comprising a composition effective to treat a hepatitis C viral infection; and packaging material comprising a label which indicates that the composition can be used to treat infection by the hepatitis C virus; wherein the composition comprises a compound according to this invention or a pharmaceutically acceptable salt thereof.

Still another aspect of this invention relates to a method of inhibiting the replication of hepatitis C virus comprising exposing the virus to an effective amount of the compound of the invention, or a salt thereof, under conditions where replication of hepatitis C virus is inhibited.

Further included in the scope of the invention is the use of a compound of the invention, or a salt thereof, to inhibit the replication of hepatitis C virus.

Yet another aspect of this invention provides a method of inhibiting HCV NS3 protease activity in a human being by administering a compound of the invention, including a pharmaceutically acceptable salt thereof.

Another aspect of this invention provides a method of decreasing the NS3 protease activity of the hepatitis C virus infecting a human being by administering a compound of the invention, including a pharmaceutically acceptable salt thereof.

### DETAILED DESCRIPTION OF PREFERRED EMBODIMENTS

### Definitions

Terms not specifically defined herein should be given the meanings that would be given to them by one of skill in the art in light of the disclosure and the context.

Unless specifically indicated, throughout the specification and the appended claims, a given chemical formula or name shall encompass salts, including pharmaceutically acceptable salts thereof and solvates thereof, such as for instance hydrates, including solvates of the free compounds or solvates of a salt of the compound. For example, the compounds of the present invention can exist in unsolvated as well as solvated forms with pharmaceutically acceptable solvents such as water, ethanol, and the like. In general, the solvated forms are considered equivalent to the unsolvated forms for the purpose of the present invention.

The term “salt thereof” as used herein is intended to mean any acid and/or base addition salt of a compound according to the invention, including but not limited to a pharmaceutically acceptable salt thereof.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. For example, such salts include acetates, ascorbates, aspartates, benzenesulfonates, benzoates, besylates, bicarbonates, bitartrates, bromides/hydrobromides, Caedetates/edetates, camsylates, carbonates, chlorides/hydrochlorides, citrates, cyclamates, edisylates, ethane disulfonates, estolates, esylates, fumarates, gentisates (salt of 2,5-dihydroxy benzoic acid), gluceptates, gluconates, glutamates, glycinates, glycolates, glycollylarsnilates, hexylresorcinates, hydrabamines, hydroxymaleates, hydroxynaphthoates, iodides, isethionates, lactates, lactobionates, malates, maleates, malonates, mandelates, methanesulfonates, mesylates, methylbromides, methylnitrates, methylsulfates, mucates, napsylates, nitrates, oxalates, pamoates, pantothenates, phenylacetates, phosphates/diphosphates, polygalacturonates, propionates, saccharinates, salicylates, stearates subacetates, succinates, sulfamides, sulfates, tannates, tartrates, teoclates, toluenesulfonates, triethiodides, xinafoates (salt of 1-hydroxy-2-naphthoicacid), ammonium, arginine, benzathines, chloroprocaines, cholines, diethanolamines, ethylenediamines, lysine, meglumines, TRIS (C,C,C-tris(hydroxymethyl)-aminomethan or Trometamol) and procaines. Further pharmaceutically acceptable salts can be formed with cations from metals like aluminium, calcium, lithium, magnesium, potassium, sodium, zinc and the like. (also see Pharmaceutical salts, Birge, S. M. et al., J. Pharm. Sci., (1977), 66, 1-19).

The pharmaceutically acceptable salts of the present invention can be synthesized from the parent compound which contains a basic or acidic moiety by conventional chemical methods. Generally, such salts can be prepared by reacting the free acid or base forms of these compounds with a sufficient amount of the appropriate base or acid in water or in an organic diluent like ether, ethyl acetate, ethanol, isopropanol, or acetonitrile, or a mixture thereof.

Salts of other acids than those mentioned above which for example are useful for purifying or isolating the compounds of the present invention also comprise a part of the invention.

The term “antiviral agent” as used herein means an agent (compound or biological) that is effective to inhibit the formation and/or replication of a virus in a human being. This includes agents that interfere with either host or viral mechanisms necessary for the formation and/or replication of a virus in a human being. Such agents can be selected from: another anti-HCV agent, HIV inhibitor, HAV inhibitor and HBV inhibitor.

As used herein, the term “treatment” means the administration of a compound or composition according to the present invention to alleviate or eliminate symptoms of the hepatitis C disease and/or to reduce viral load in a patient.

As used herein, the term “prevention” means the administration of a compound or composition according to the present invention post-exposure of the individual to the virus but before the appearance of symptoms of the disease, and/or prior to the detection of the virus in the blood, to prevent the appearance of symptoms of the disease.

The term “therapeutically effective amount” means an amount of a compound according to the invention, which when administered to a patient in need thereof, is sufficient to effect treatment for disease-states, conditions, or disorders for which the compounds have utility. Such an amount would be sufficient to elicit the biological or medical response of a tissue system, or patient that is sought by a researcher or clinician. The amount of a compound according to the invention which constitutes a therapeutically effective amount will vary depending on such factors as the compound and its biological activity, the composition used for administration, the time of administration, the route of administration, the rate of excretion of the compound, the duration of the treatment, the type of disease-state or disorder being treated and its severity, drugs used in combination with or coincidentally with the compounds of the invention, and the age, body weight, general health, sex and diet of the patient. Such a therapeutically effective amount can be determined routinely by one of ordinary skill in the art having regard to their own knowledge, the state of the art, and this disclosure.

### Pharmaceutical Composition

Suitable preparations for administering the compounds of the invention will be apparent to those with ordinary skill in the art and include for example tablets, pills, capsules, suppositories, lozenges, troches, solutions, syrups, elixirs, sachets, injectables, inhalatives and powders. The content of the pharmaceutically active compound(s) should be in the range from 0.05 to 90 wt.-%, preferably 0.1 to 50 wt.-% of the composition as a whole.

Suitable tablets may be obtained, for example, by mixing one or more compounds according to the invention with known excipients, for example inert diluents, carriers, disintegrants, adjuvants, surfactants, binders and/or lubricants. The tablets may also consist of several layers.

According to an alternate embodiment, the pharmaceutical composition of this invention may additionally comprise at least one other anti-HCV agent.

The term “other anti-HCV agent” as used herein means those agents that are effective for diminishing or preventing the progression of hepatitis C related symptoms of disease. Such agents can be selected from: immunomodulatory agents, inhibitors of HCV NS3 protease, inhibitors of HCV polymerase or inhibitors of another target in the HCV life cycle. Examples of anti-HCV agents include, α—(alpha), β—(beta), δ—(delta), γ—(gamma), ω—(omega) or τ—(tau) interferon, pegylated α-interferon, ribavirin, amantadine, taribavirin (Viramidine), Nitazoxannide and BMS-791325.

The term “immunomodulatory agent” as used herein includes those agents (compounds or biologicals) that are effective to enhance or potentiate the immune system response in a human being. Immunomodulatory agents include, but are not limited to, inosine monophosphate dehydrogenase inhibitors, class I interferons, class II interferons, consensus interferons, asialo-interferons pegylated interferons and conjugated interferons, including but not limited to interferons conjugated with other proteins including but not limited to human albumin. Class I interferons are a group of interferons that all bind to receptor type I, including both naturally and synthetically produced class I interferons, while class II interferons all bind to receptor type II. Examples of class I interferons include, but are not limited to, α-, β-, δ-, ω-, and τ-interferons, while examples of class II interferons include, but are not limited to, γ-interferons.

The term “inhibitor of HCV NS3 protease” as used herein means an agent (compound or biological) that is effective to inhibit the function of HCV NS3 protease in a human being. Inhibitors of HCV NS3 protease include, for example, those compounds described in WO 99/07733, WO 99/07734, WO 00/09558, WO 00/09543, WO 00/59929, WO 03/064416, WO 03/064455, WO 03/064456, WO 2004/039970, WO 2004/037855, WO 2004/039833, WO 2004/101602, WO 2004/101605, WO 2004/103996, WO 2005/028501, WO 2005/070955, WO 2006/000085, WO 2006/007700, WO 2006/007708, WO 2007/009227, WO 2004/093915, WO 2004/009121 (all by Boehringer Ingelheim), all of which are herein incorporated by reference; and the candidates ABT-450, ACH-1625, BMS-650032, PHX1766, VX-813, VX-950, AVL-181, SCH-503034, SCH-900518, ITMN-191, TMC 435350, MK7009 and BI 201335.

The term “inhibitor of HCV polymerase” as used herein means an agent (compound or biological) that is effective to inhibit the function of an HCV polymerase in a human being. This includes, for example, inhibitors of HCV NS5B polymerase.

Inhibitors of HCV polymerase include for example, those compounds described in: WO 03/007945, WO 03/010140, WO 03/010141, U.S. Pat. No. 6,448,281, WO 02/04425, WO 2008/019477, WO 2007/087717, WO 2006/007693, WO 2005/080388, WO 2004/099241, WO 2004/065367, WO 2004/064925 (all by Boehringer Ingelheim), all of which are herein incorporated by reference. Specific examples of inhibitors of an HCV polymerase, include RG-7128, GS9190, IDX184, PSI-7851, MK-3281, PF868554, VCH-222, VCH-759, ANA598, ABT-333 and ABT-072.

The term “inhibitor of another target in the HCV life cycle” as used herein means an agent (compound or biological) that is effective to inhibit the formation and/or replication of HCV in a human being other than by inhibiting the function of the HCV NS3 protease. This includes agents that interfere with either host or HCV viral targets necessary for the HCV life cycle or agents which specifically inhibit in HCV cell culture assays through an undefined or incompletely defined mechanism.

Inhibitors of another target in the HCV life cycle include, for example, agents that inhibit viral targets such as Core, E1, E2, p7, NS2/3 protease, NS3 helicase, NS4A, NS5A, NS5B polymerase, and internal ribosome entry site (IRES), or host targets such as cyclophilin B, phosphatidylinositol 4-kinase IIIα, CD81, SR-B1, Claudin 1, VAP-A, VAP-B. Specific examples of inhibitors of another target in the HCV life cycle include SCY-635, ITX5061, NOV-205, AZD7295, BIT-225, NA808, MK-1220, PF-4878691, MX-3253, GS 9450, BMS-790052, ISIS-14803, GS9190, NIM-811, and DEBIO-025.

The term “HIV inhibitor” as used herein means an agent (compound or biological) that is effective to inhibit the formation and/or replication of HIV in a human being. This includes agents that interfere with either host or viral mechanisms necessary for the formation and/or replication of HIV in a human being. HIV inhibitors include, for example, nucleoside inhibitors, non-nucleoside inhibitors, protease inhibitors, fusion inhibitors and integrase inhibitors.

The term “HAV inhibitor” as used herein means an agent (compound or biological) that is effective to inhibit the formation and/or replication of HAV in a human being. This includes agents that interfere with either host or viral mechanisms necessary for the formation and/or replication of HAV in a human being. HAV inhibitors include Hepatitis A vaccines, for example, Havrix® (GlaxoSmithKline), VAQTA® (Merck) and Avaxim® (Aventis Pasteur).

The term “HBV inhibitor” as used herein means an agent (compound or biological) that is effective to inhibit the formation and/or replication of HBV in a human being. This includes agents that interfere with either host or viral mechanisms necessary for the formation and/or replication of HBV in a human being. HBV inhibitors include, for example, agents that inhibit HBV viral DNA polymerase or HBV vaccines. Specific examples of HBV inhibitors include Lamivudine (Epivir-HBV®), Adefovir Dipivoxil, Entecavir, FTC (Coviracil®), DAPD (DXG), L-FMAU (Clevudine®), AM365 (Amrad), Ldt (Telbivudine), monoval-LdC (Valtorcitabine), ACH-126,443 (L-Fd4C) (Achillion), MCC478 (Eli Lilly), Racivir (RCV), Fluoro-L and D nucleosides, Robustaflavone, ICN 2001-3 (ICN), Bam 205 (Novelos), XTL-001 (XTL), Imino-Sugars (Nonyl-DNJ) (Synergy), HepBzyme; and immunomodulator products such as: interferon alpha 2b, HE2000 (Hollis-Eden), Theradigm (Epimmune), EHT899 (Enzo Biochem), Thymosin alpha-1 (Zadaxin®), HBV DNA vaccine (PowderJect), HBV DNA vaccine (Jefferon Center), HBV antigen (OraGen), BayHep B® (Bayer), Nabi-HB® (Nabi) and Anti-hepatitis B (Cangene); and HBV vaccine products such as the following: Engerix B, Recombivax HB, GenHevac B, Hepacare, Bio-Hep B, TwinRix, Comvax, Hexavac.

Specific preferred examples of some of these agents are listed below:
antiviral agents: ribavirin or amantadine;
immunomodulatory agents: class I interferons, class II interferons or pegylated forms thereof;
HCV polymerase inhibitors: nucleoside analogs or non-nucleosides;
inhibitor of another target in the HCV life cycle that inhibits a target selected from: NS3 helicase, N52/3 protease, internal ribosome entry site (IRES), NS4A, NS5A, NS5B polymerase, or host targets such as cyclophilin A or B;
HIV inhibitors: nucleosidic inhibitors, non-nucleosidic inhibitors, protease inhibitors, fusion inhibitors or integrase inhibitors; or
HBV inhibitors: agents that inhibit viral DNA polymerase or is an HBV vaccine.

As discussed above, combination therapy is contemplated wherein a compound of the invention, or a pharmaceutically acceptable salt thereof, is co-administered with at least one additional agent selected from: an antiviral agent, an immunomodulatory agent, another inhibitor of HCV NS3 protease, an inhibitor of HCV polymerase, an inhibitor of another target in the HCV life cycle, an HIV inhibitor, an HAV inhibitor and an HBV inhibitor. These additional agents may be combined with the compounds of this invention to create a single pharmaceutical dosage form. Alternatively these additional agents may be separately administered to the patient as part of a multiple dosage form, for example, using a kit. Such additional agents may be administered to the patient prior to, concurrently with, or following the administration of a compound of the invention, or a pharmaceutically acceptable salt thereof.

According to another alternate embodiment, the pharmaceutical composition of this invention may additionally comprise at least one other inhibitor of HCV NS3 protease.

According to another alternate embodiment, the pharmaceutical composition of this invention may additionally comprise at least one inhibitor of HCV polymerase.

According to yet another alternate embodiment, the pharmaceutical composition of this invention may additionally comprise at least one inhibitor of other targets in the HCV life cycle, including but not limited to, helicase, NS5A protease, NS2/3 protease or internal ribosome entry site (IRES).

The dose range of the compounds of the invention applicable per day is usually from 0.01 to 100 mg/kg of body weight, preferably from 0.1 to 50 mg/kg of body weight. Each dosage unit may conveniently contain from 5% to 95% active compound (w/w). Preferably such preparations contain from 20% to 80% active compound.

The actual pharmaceutically effective amount or therapeutic dosage will of course depend on factors known by those skilled in the art such as age and weight of the patient, route of administration and severity of disease. In any case the combination will be administered at dosages and in a manner which allows a pharmaceutically effective amount to be delivered based upon patient's unique condition.

When the composition of this invention comprises a combination of a compound of the invention and one or more additional therapeutic or prophylactic agent, both the compound and the additional agent should be present at dosage levels of between about 10 to 100%, and more preferably between about 10 and 80% of the dosage normally administered in a monotherapy regimen.

### EXAMPLES

Temperatures are given in degrees Celsius. Solution percentages express a weight to volume relationship, and solution ratios express a volume to volume relationship, unless stated otherwise. Retention times (t_{R}) for each compound are measured using the standard analytical HPLC conditions described in the Examples. As is well known to one skilled in the art, retention time values are sensitive to the specific measurement conditions. Therefore, even if identical conditions of solvent, flow rate, linear gradient, and the like are used, the retention time values may vary when measured, for example, on different HPLC instruments. Even when measured on the same instrument, the values may vary when measured, for example, using different individual HPLC columns, or, when measured on the same instrument and the same individual column, the values may vary, for example, between individual measurements taken on different occasions.

Abbreviations used in the examples include:

Ac: acetyl; ACCA: 1-Aminocyclopropyl-carboxylic acid; BOC or Boc: tert-butyloxycarbonyl; DCM: dichloromethane; DIAD: diisopropylazodicarboxylate; DIPEA: diisopropylethylamine; DMF: N,N-dimethylformamide; DMSO: dimethylsulfoxide; equiv: equivalent; Et: ethyl; EtOAc: ethyl acetate; HATU: [O-7-azabenzotriazol-1-yl)-1,1,3,3-tetramethyluronium hexafluorophosphate]; hex: hexanes; Het: heterocycle; HPLC: high performance liquid chromatography; LiHMDS: lithium bis(trimethylsilyl) amide; M: mole/liter; Me: methyl; MeOH: methanol; mins: minutes; mmol: millimole; MS: mass spectrometry (FIA MS—flow injection analysis mass spectrometry; HPLC: Ultraperformance Liquid Chromatography); NMP: N-methylpyrrolidinone; NMR: nuclear magnetic resonance; Ph: phenyl; prep HPLC: preparative high performance liquid chromatography; RT: room temperature (18 to 22° C.); sat: saturated; SM: starting material; SNAr: Nucleophilic aromatic substitution; tert-butyl or t-butyl: 1,1-dimethylethyl; t-BME: tert-butyl methyl ether; TBTU: 2-(1H-benzotriazole-1-yl)-1,1,3,3-tetramethyl uronium tetrafluoroborate; TEA: triethylamine; TFA: trifluoroacetic acid; and THF: tetrahydrofuran.

NMR: Chemical shifts are reported in parts per million from tetramethylsilane with the solvent resonance as the internal standard. Data are reported as follows: chemical shift, multiplicity (s=singlet, d=doublet, t=triplet, q=quartet, qn=quintet, S=septuplet, m=multiplet and b=broad), integration and coupling constant in Hz. Flash chromatography is carried out on silica gel (SiO₂) according to Still's flash chromatography technique (W. C. Still et al., J. Org. Chem. 1978, 43, 2923). Alternatively compounds and intermediates can be purified on a Teledyne ISCO Combiflash R₁ System at 254 nm using commercial normal phase silica 4-120 g Redisep Rf or Silicycle columns eluting in 0-100% EtOAc/Hexane or 0-10% MeOH/DCM at a flow rate of 18-85 mL/min depending on column size. Mass spectral analyses are recorded using flow injection analysis mass spectrometry or Waters Acquity Ultraperformance LC System consisting of a sample organizer, PDA detector, column manager, sample manager, binary solvent manager and SQ detector. Analytical HPLC is carried out under standard conditions using a SunFire™ C18 3.5 μM reverse phase column, 4.6×30 mm and a linear gradient (0 to 100% over 8 mins with 2.5 mL/min) employing 0.1% TFA/acetonitrile and 0.1% TFA/water as solvents. Preparative chromatography purification is carried out using a Waters Autopurify Chromatography System consisting of the following components: 1) Sample Manager Model 2767; 2) Pump Model 2525 or 2545); 3) PDA Detector Model 2996 or 2998; 4) System Fluidics Organizer (SFO) or Column Fluidics Organizer (CFO) with or without the additional component 5) Mass Spec Model 3100.

### Purification Columns:

Sunfire Prep C₁₈ Column OBD; 19×50 mm, 5 μM (Part No. 186002566) using a 0-100% Gradient at 30 mL/min 0.1% TFA/Acetonitrile or Ammonium Formate/MeOH at pH 3.8 (for Ammonium Formate Gradient Conditions: see below).

X-Bridge Prep C₁₈ Column OBD; 19×50 mm, 5 μM (Part No. 186002977) using Ammonium Bicarbonate/MeOH at pH 10; (for Gradient Conditions: see below).

Gradient Program for Ammonium Formate or Ammonium Bicarbonate (A)/MeOH (B) are as follows:

At 30 mL/min:

| Time, min | % A | % B | Curve |
|---|---|---|---|
| 0 | 100 − X | X | — |
| 1.00 | 100 − X | X | 6 |
| 11.00 | 100 − (X + 20) | X + 20 | 6 |
| 11.10 | 0 | 100 | 6 |
| 13.10 | 0 | 100 | 6 |

where X is a predetermined value dependant on the obtained analytical HPLC retention time particular to each product.

### Methodology

Compounds 1001-1029 of the present invention are prepared according to the general procedures described in Scheme 1 and/or Scheme 2.

### General Scheme 1

In Scheme 1, the Boc protected amines Aa-h and Ba-h are deprotected under standard acidic conditions to provide Ca-h and Da-h. A coupling reaction with the corresponding heterocyclic acids or sodium salts R2a-n from Table A1 and appropriate reagents provide the desired products.

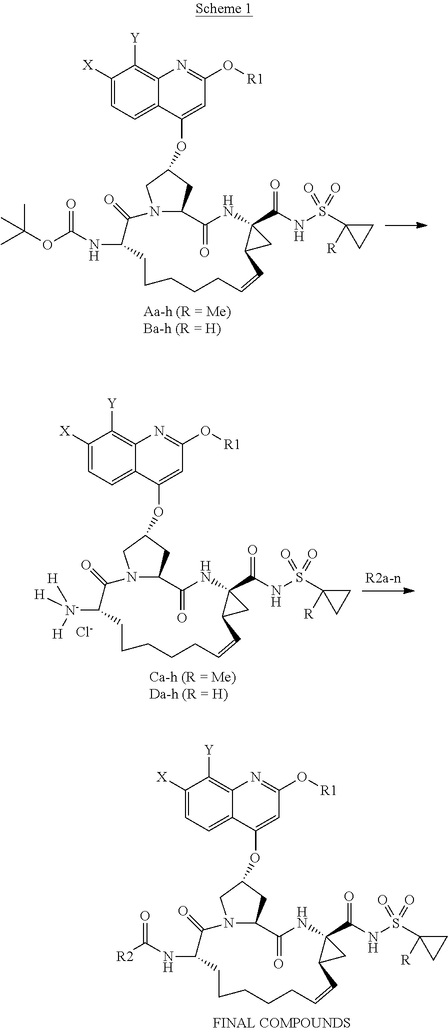

### General Scheme 2

Alternatively, in Scheme 2, the appropriate heterocyclic capping group (R2a-n from Table A1) is installed first on the macrocyclic brosylates (E and/or F) after removal of the Boc-protecting group under standard acidic conditions and then appropriately substituted quinoline (Qa-h from Table A2) is installed via S_{N}Ar to provide the desired products.

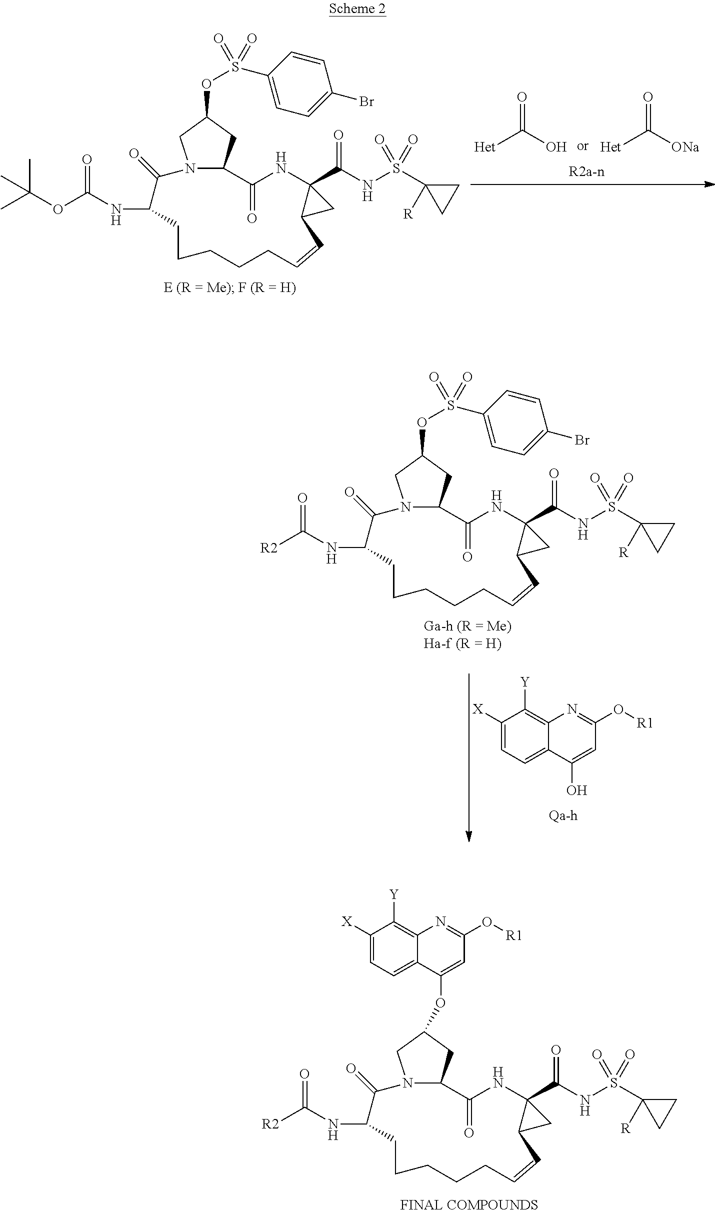

### Definition of Intermediates

### Capping Groups R

TABLE A1
Definition of R2a-R2n

| | |
|---|---|
| 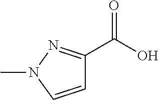 | R2a |
| 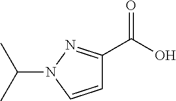 | R2b |
| 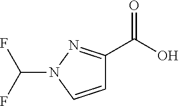 | R2c |
| 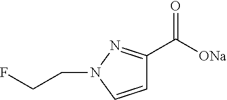 | R2d |
| 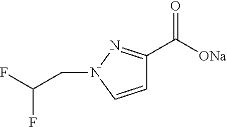 | R2e |
| 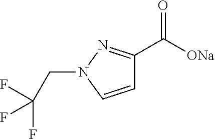 | R2f |
| 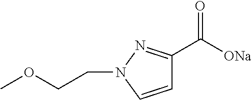 | R2g |
| 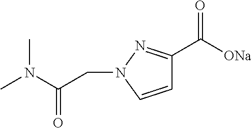 | R2h |
| 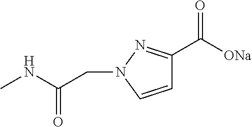 | R2i |
| 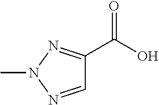 | R2j |
| 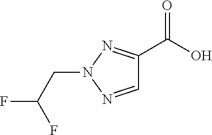 | R2k |
| 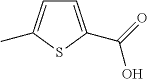 | R2l |
| 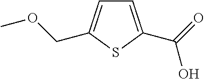 | R2m |
| 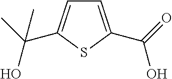 | R2n |

### Quinolines Q

TABLE A2
Definition of Qa-Qh

| | |
|---|---|
| 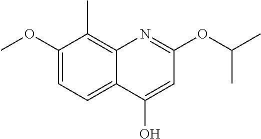 | Qa |
| 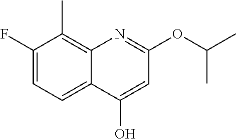 | Qb |
| 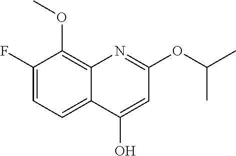 | Qc |
| 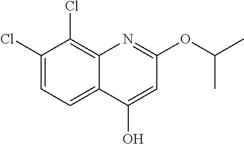 | Qd |
| 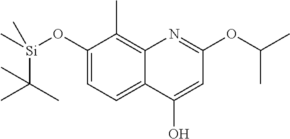 | Qe |
| 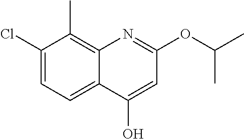 | Qf |
| 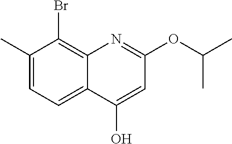 | Qg |
| 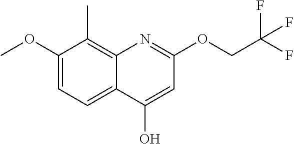 | Qh |

### Macrocyclic Intermediates A and B

The synthesis of Aa-h and Ba-h is realized by utilizing the general procedures to described in either Scheme 3 and/or Scheme 4. In Scheme 3, the common intermediate I is submitted to S_{N}Ar conditions to incorporate the appropriately substituted quinolines (Qa-h). These intermediates are then converted to the corresponding acylsulfonamides Aa-h and Ba-h via the azalactones La-h through well documented procedures, such as WO 2006/000085, WO 2006/007700, WO 2006/007708, WO 2007/014922, Heterocycles 2009, 79, 985-1005, Synthesis 2009, 4, 620-626 and European Journal of Medicinal Chemistry 2009, 44(2), 891-900.

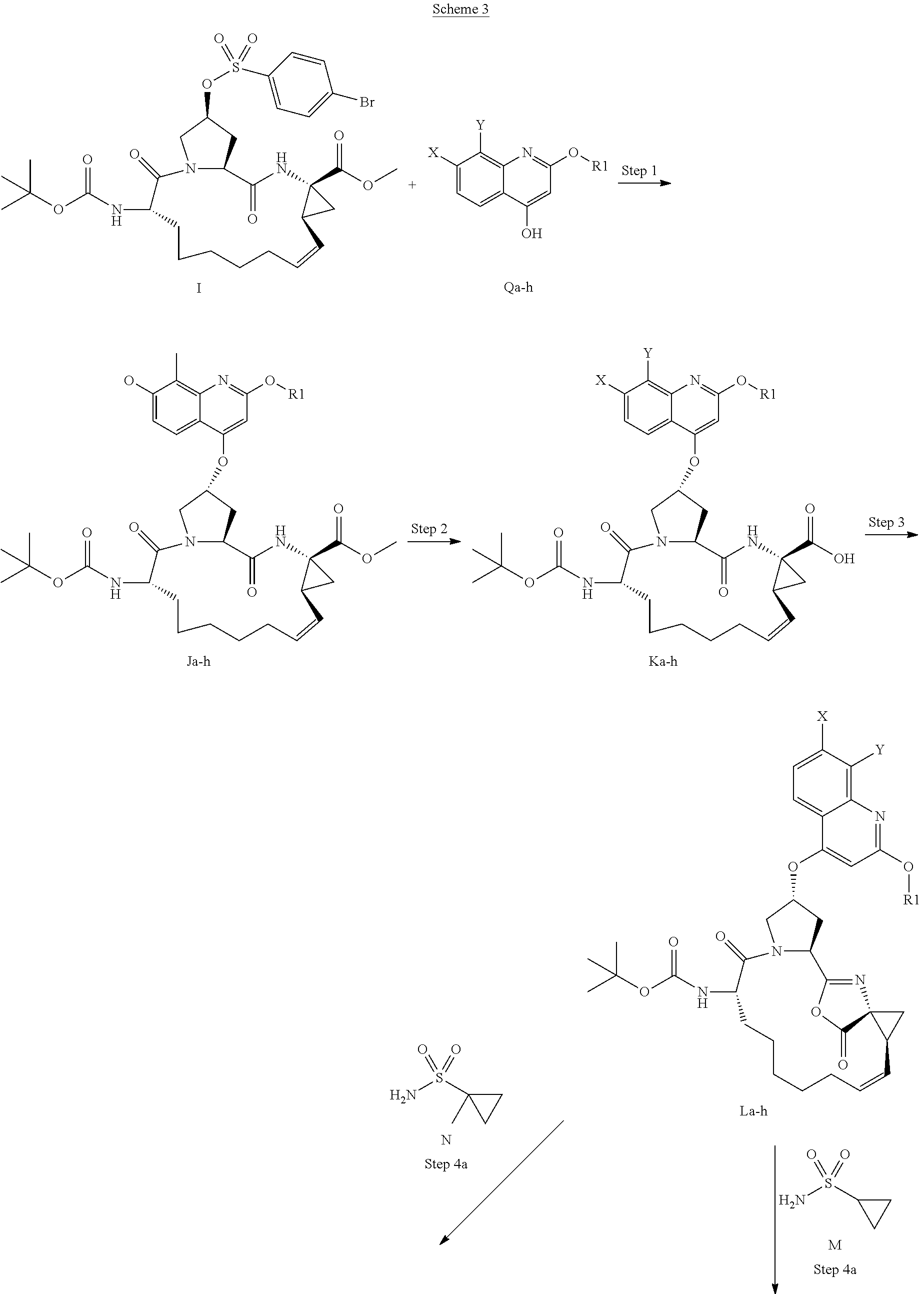

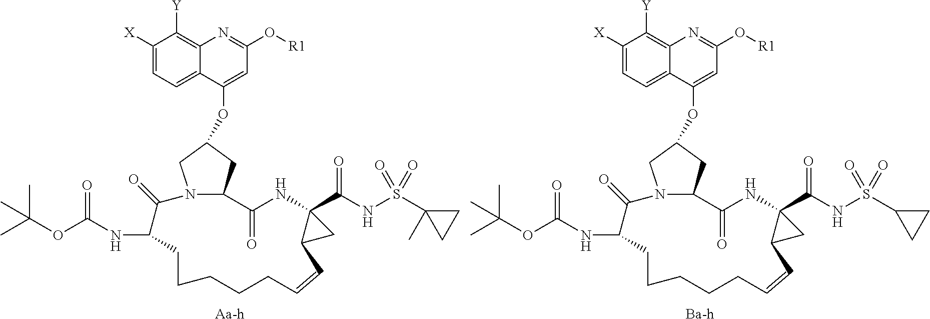

Alternatively, in Scheme 4, the azalactone O is formed first from the brosylate intermediate I which is then reacted with sulfonamides M or N to form intermediates E or F followed by incorporation of the appropriately substituted hydroxyquinoline (Qa-Qh) via SNAr.

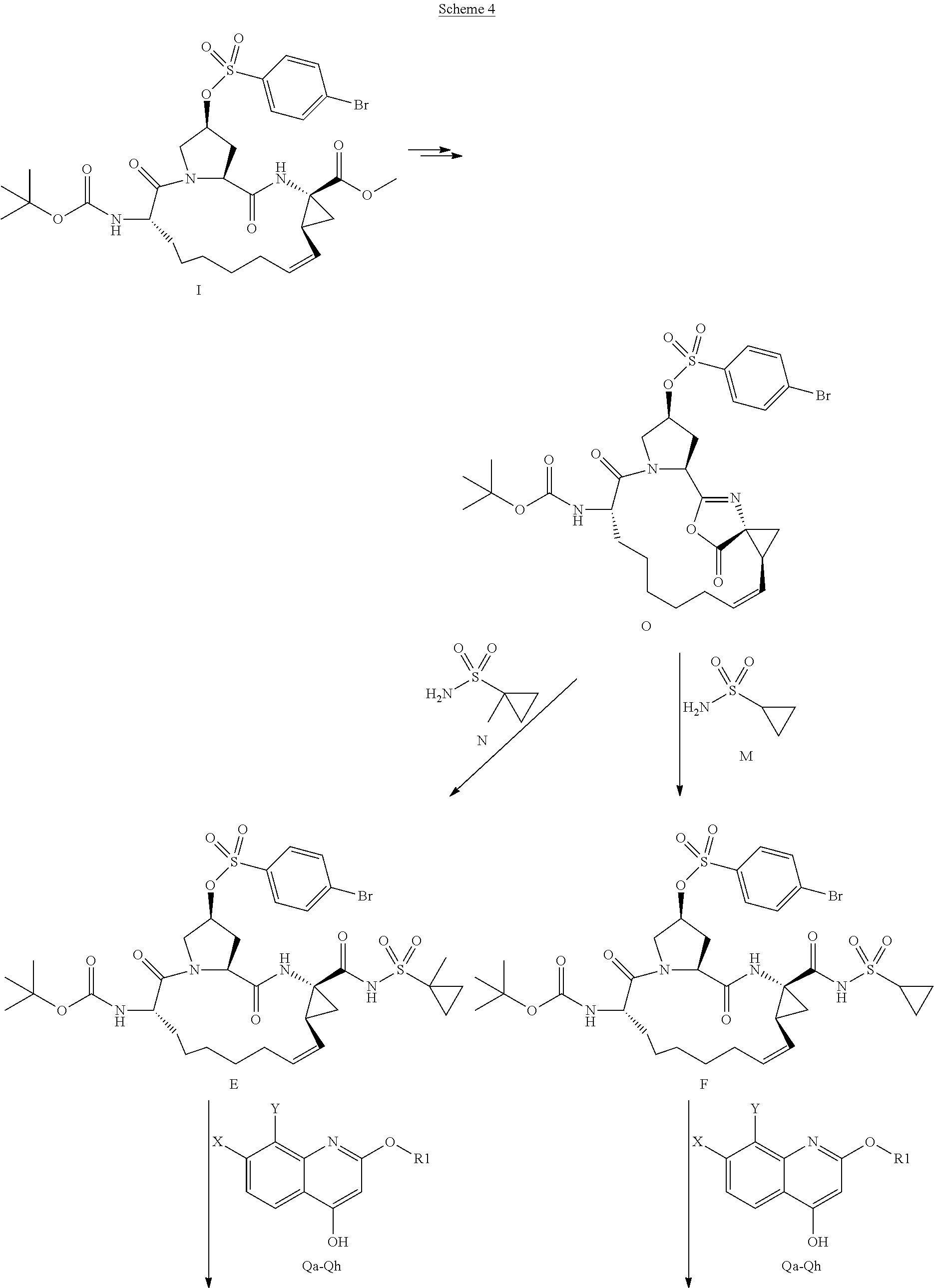

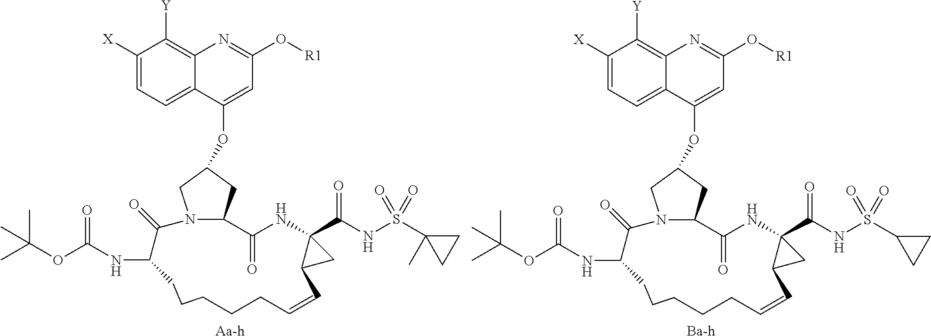

### Synthesis of Intermediates

### Synthesis of Capping Groups R2a-n (from Table A1)

R2a, R2b, R2c, R2f, R2l and R2m are available from commercial sources and are used as received without further purification. (Commercial sources: R2a, R2m: Chembridge BB; R2b, R2c: Artchem BB; R2f, AKOS; R2l: Aldrich)

### Synthesis of R2d,e,g

The synthesis is done as described in scheme 5:

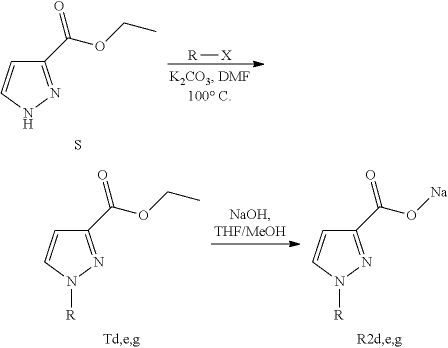

### Synthesis of Sodium 1-(2-fluoro-ethyl)-1H-pyrazole-3-carboxylate (R2d)

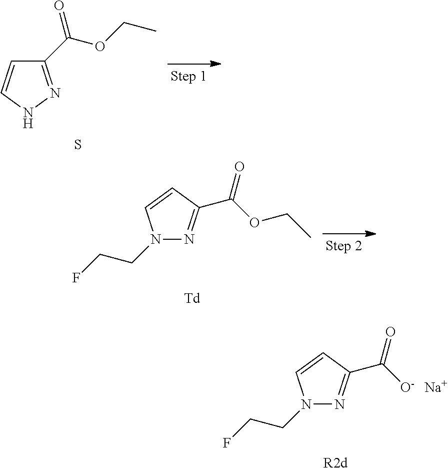

### Step 1

To a solution of 1H-pyrazole-3-carboxylic acid ethyl ester S (500 mg, 3.57 mmol) in DMF (6.5 mL) is added K₂CO₃ (542 mg, 3.93 mmol, 1.10 equiv), NaI (1.07 g, 7.14 mmol, 2 equiv) and 1-bromo-2-fluoroethane (928 mg, 7.14 mmol, 2.00 equiv). The reaction mixture is stirred at 100° C. for 48 h in a closed vial. The reaction is quenched with HCl 1 N (pH ˜5-6), water is added and the mixture is extracted with EtOAc (5×). The organics are washed with brine, dried with anhydrous MgSO₄, filtered and concentrated. The crude mixture containing the 2 regioisomers is purified by prep HPLC. The appropriate fractions are combined, frozen and lyophilized to give Td.

UPLC-MS: 186.8 (M+H)⁺

¹H NMR (400 MHz, DMSO-d₆) δ (ppm): 7.90 (d, J=2.4 Hz, 1H), 6.76 (d, J=2.4 Hz, 1H), 4.79 (dt, J=47.3, 4.7 Hz, 2H), 4.53 (dt, J=27.8, 4.7 Hz, 2H), 4.26 (q, J=7.1 Hz, 2H), 1.28 (t, J=7.1 Hz, 3H).

### Step 2

1-(2-Fluoro-ethyl)-1H-pyrazole-3-carboxylic acid ethyl ester (Td) (337 mg, 1.81 mmol) is dissolved in THF/MeOH (6 mL, 3:1 mixture). NaOH 1 M (1.99 mL, 1.99 mmol, 1.10 equiv) is added and the reaction is stirred at RT for ˜12 h. The crude mixture is evaporated to dryness, diluted in H₂O/MeCN, frozen and lyophilized to give R2d.

UPLC-MS: 158.9 (M+H)⁺ (for the corresponding acid)

¹H NMR (400 MHz, DMSO-d₆) δ (ppm): 7.58 (d, J=1.9 Hz, 1H), 6.33 (d, J=1.9 Hz, 1H), 4.75 (dt, J=47.3, 4.7 Hz, 2H), 4.41 (dt, J=27.4, 4.7 Hz, 2H).

### Synthesis of Sodium 1-(2,2-difluoro-ethyl)-1H-pyrazole-3-carboxylate (R2e)

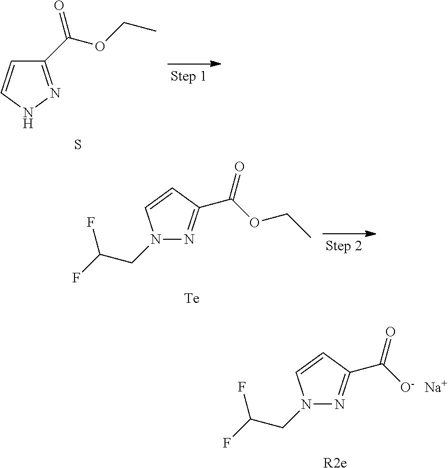

### Step 1

Compound Te is prepared analogously to the procedure described for the preparation of 1-(2-Fluoro-ethyl)-1H-pyrazole-3-carboxylic acid ethyl ester (Td) using 1H-pyrazole-3-carboxylic acid ethy ester S (300 mg, 2.14 mmol) and 1,1-difluoro-2-iodoethane (150 μL, 1.70 mmol, 0.80 equiv) as the alkylating agent. NaI is not added. The crude mixture containing the 2 regioisomers is purified by prep HPLC. The appropriate fractions are combined, frozen and lyophilized to give Te.

UPLC-MS: 191.1 (M+H)⁺

¹H NMR (400 MHz, DMSO-d₆) δ (ppm): 7.92 (d, J=2.3 Hz, 1H), 6.80 (d, J=2.3 Hz, 1H), 6.41 (tt, J=54.6, 3.8 Hz, 1H), 4.75 (td, J=15.1, 3.8 Hz, 2H), 4.27 (q, J=7.1 Hz, 2H), 1.28 (t, J=7.1 Hz, 3H).

### Step 2

Compound R2e is synthesized analogously to the procedure for the hydrolysis of sodium 1-(2-fluoro-ethyl)-1H-pyrazole-3-carboxylate (R2d) using 166 mg (0.82 mmol) of Te.

UPLC-MS: 176.9 (M+H)⁺ (for the corresponding acid)

¹H NMR (400 MHz, DMSO-d₆) δ (ppm): 7.61 (d, J=2.3 Hz, 1H), 6.35 (d, J=2.3 Hz, 1H), 6.35 (tt, J=55.5, 4.1 Hz, 1H), 4.55 (td, J=14.7, 4.1 Hz, 2H).

### Synthesis of Sodium 1-(2-methoxy-ethyl)-1H-pyrazole-3-carboxylate (R2g)

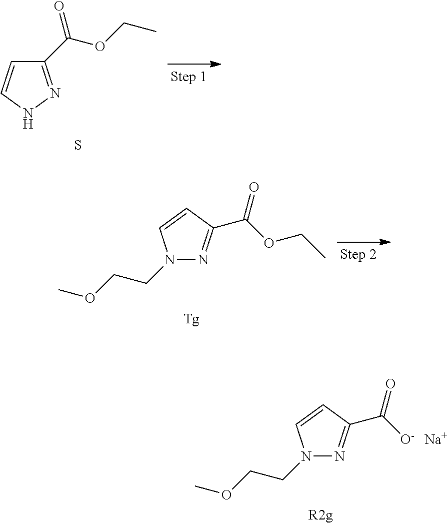

### Step 1

Compound Tg is synthesized analogously to the procedure used for the preparation of 1-(2-Fluoro-ethyl)-1H-pyrazole-3-carboxylic acid ethyl ester (Td) using 1H-pyrazole-3-carboxylic acid ethyl ester S (500 mg, 3.57 mmol) and 2-bromomethyl methyl ether (685 μL, 7.28 mmol, 2.00 equiv) as the alkylating agent. The crude mixture containing the 2 regioisomers is purified by prep HPLC. The appropriate fractions are combined, frozen and lyophilized to give Tg.

UPLC-MS: 199.4 (M+H)⁺

¹H NMR (400 MHz, DMSO-d₆) δ (ppm): 7.83 (d, J=2.3 Hz, 1H), 6.72 (d, J=2.3 Hz, 1H), 4.34 (t, J=5.3 Hz, 2H), 4.26 (q, J=7.1 Hz, 2H), 3.69 (t, J=5.3 Hz, 2H), 3.22 (s, 3H), 1.28 (t, J=7.1 Hz, 3H).

### Step 2

Compound R2g is synthesized analogously to the procedure for the hydrolysis of sodium 1-(2-fluoro-ethyl)-1H-pyrazole-3-carboxylate (R2d) using 426 mg (2.15 mmol) of Tg.

UPLC-MS: 171.1 (M+H)⁺ (for the corresponding acid)

¹H NMR (400 MHz, DMSO-d₆) δ (ppm): 7.52 (d, J=2.4 Hz, 1H), 6.30 (d, J=2.4 Hz, 1H), 4.21 (t, J=5.5 Hz, 2H), 3.65 (t, J=5.5 Hz, 2H), 3.21 (s, 3H).

### Synthesis of R2h and R2i

The synthesis of these compounds is done according to scheme 6:

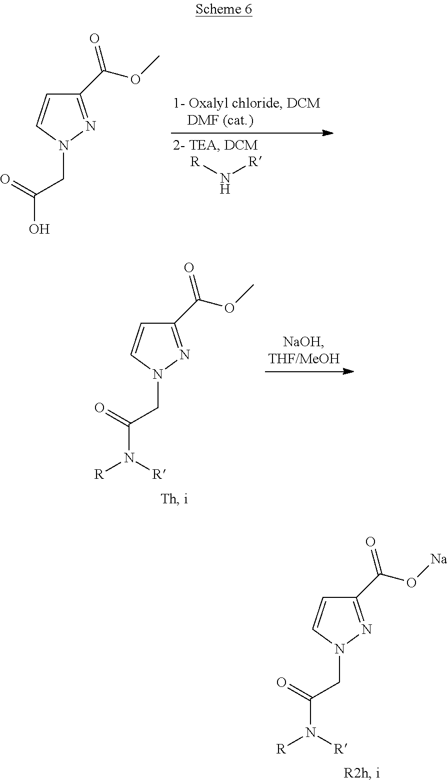

### Synthesis of Sodium 1-dimethylcarbamoylmethyl-1H-pyrazole-3-carboxylate (R2h)

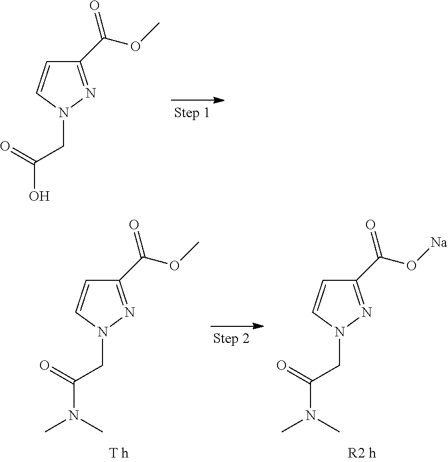

### Step 1

To a solution of 1-carboxymethyl-1H-pyrazole-3-carboxylic acid methyl ester (200 mg, 1.09 mmol) in DCM (3.6 mL) at 0° C. is added DMF (10 μL, 0.13 mmol, 0.12 equiv) followed by oxalyl chloride 2 M in DCM (0.71 mL, 1.41 mmol, 1.30 equiv). The reaction is stirred at RT for 2 h. The reaction mixture is evaporated to dryness and the acyl chloride is used as such. To the acyl chloride (220 mg, 1.09 mmol) in DCM (3.6 mL) is added dimethylamine 2 M in THF (0.81 mL, 1.63 mmol, 1.50 equiv) and TEA (0.45 mL, 3.26 mmol, 3.00 equiv). The reaction is stirred at RT for 12 h.

The crude mixture is evaporated to dryness and purified by flash chromatography to give Th.

UPLC-MS: 212.1 (M+H)⁺

¹H NMR (400 MHz, DMSO-d₆) δ (ppm): 7.76 (d, J=2.3 Hz, 1H), 6.75 (d, J=2.3 Hz, 1H), 5.23 (s, 2H), 3.78 (s, 3H), 3.03 (s, 3H), 2.85 (s, 3H).

### Step 2

Compound R2h is made analogously to the procedure used for the hydrolysis of sodium 1-(2-fluoro-ethyl)-1H-pyrazole-3-carboxylate (R2d) using 179 mg (0.85 mmol) of Th.

UPLC-MS: 197.9 (M+H)⁺ (for the corresponding acid)

¹H NMR (400 MHz, DMSO-d₆) δ (ppm): 7.43 (d, J=2.2 Hz, 1H), 6.30 (d, J=2.2 Hz, 1H), 5.04 (s, 2H), 3.02 (s, 3H), 2.85 (s, 3H).

### Synthesis of Sodium 1-methylcarbamoylmethyl-1H-pyrazole-3-carboxylate (R2i)

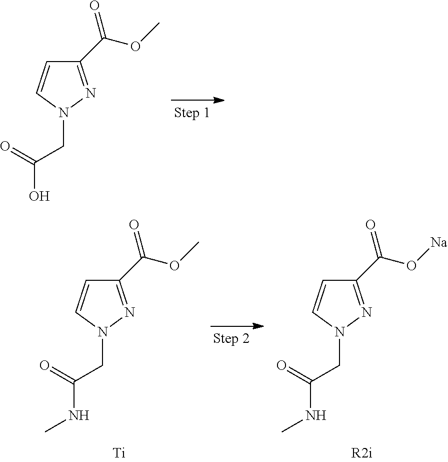

### Step 1

Compound Ti is made analogously to the procedure used for the preparation of 1-dimethylcarbamoylmethyl-1H-pyrazole-3-carboxylic acid methyl ester (Th) using methyl amine 2 M in THF (0.81 mL, 1.63 mmol, 1.30 equiv) to form the amide. The crude mixture is evaporated to dryness and purified by flash chromatography to give Ti.

HPLC-MS: 197.9 (M+H)⁺

¹H NMR (400 MHz, DMSO-d₆) δ (ppm): 8.10 (b, 1H), 7.83 (d, J=2.3 Hz, 1H), 6.75 (d, J=2.3 Hz, 1H), 4.86 (s, 2H), 3.78 (s, 3H), 2.62 (d, J=4.3 Hz, 3H).

### Step 2

Compound R2I is made analogously to the procedure used for the hydrolysis of sodium 1-(2-fluoro-ethyl)-1H-pyrazole-3-carboxylate (R2d) using 171 mg (0.87 mmol) of Ti.

UPLC-MS: 184.1 (M+H)⁺ (for the corresponding acid)

¹H NMR (400 MHz, DMSO-d₆) δ (ppm): 8.08 (b, 1H), 7.51 (d, J=1.9 Hz, 1H), 6.31 (d, J=1.9 Hz, 1H), 2.15 (s, 2H), 2.61 (d, J=4.7 Hz, 3H).

### Synthesis of 2-methyl 2H-1,2,3-triazole 4-carboxylic acid (R2j)

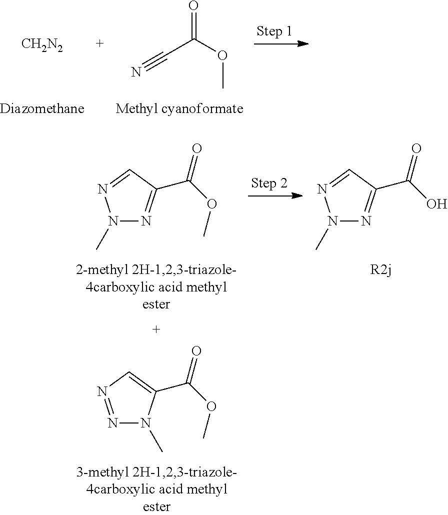

### Step 1

Methyl cyanoformate (1.00 g, 11.7 mmol) is charged in a flask, dissolved in THF (40 mL), then a 0.6 M diazomethane solution in Et₂O (58.8 mL, 35.3 mmol, 3.00 equiv) is added. This solution is stirred at RT for 16 h. Water (40 mL) and EtOAc (40 mL) are added and then the layers are separated. The solvent is evaporated and purification is performed on Combiflash (20-100% hexane) to provide the 3,4-regioisomer and the desired 2,4-regioisomer methyl esters.

2,4-regioisomer: FIA M.S. (electrospray): 142.2 (M+H)⁺

¹H NMR (400 MHz, CDCl₃): δ 8.05 (s, 1H), 4.28 (s, 3H) 3.96 (s, 3H).

3,4-regioisomer:'H NMR (400 MHz, CDCl₃): δ 8.14 (s, 1H), 4.35 (s, 3H) 3.95 (s, 3H).

### Step 2

2-methyl 2H-1,2,3-triazole-4-carboxylic acid methyl ester (263 mg, 1.86 mmol) is charged in a round-bottom flask, then THF (15 mL), 1 M solution NaOH (9.3 mL, 9.3 mmol, 5.0 equiv) and MeOH (5 mL) are measured and mixed in a graduated cylinder, then added to the flask. The solution is stirred at RT. After 4 h, 1 M HCl is added (10 mL) and the solvent is evaporated. EtOAc is added and the layers are separated. The solvent is evaporated which affords the product R2j.

¹H NMR (400 MHz, DMSO-d₆): δ 8.17 (s, 1H), 4.22 (s, 3H).

### Synthesis of 2-(2,2-difluoro-ethyl)-2H-1,2,3-triazole-4-carboxylic acid (R2k)

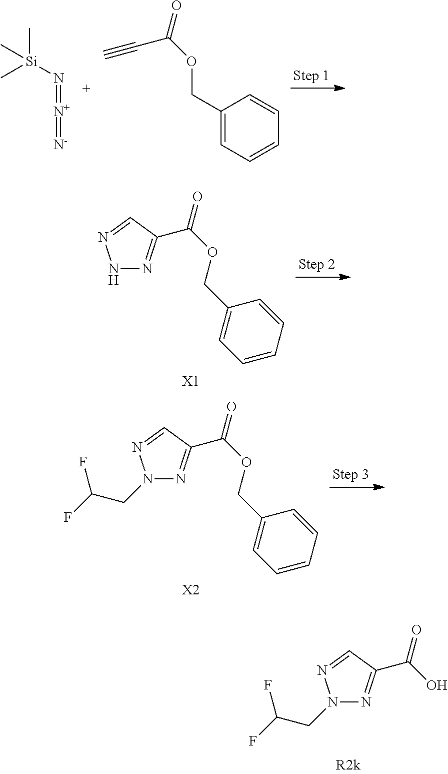

### Step 1

In a 150 mL sealed flask is added benzylated propiolic acid (1.00 g, 6.24 mmol) and azidotrimethylsilane (2.20 mL, 15.6 mmol) in MeOH (2 mL). The mixture is warmed at 80° C. for 16 h. The mixture is then concentrated and purified by CombiFlash eluting with hexane/EtOAc (15% to 100%) to afford triazole X1.

### Step 2

In a flask is charged triazole X1 (300 mg, 1.48 mmol), 2,2-difluoroethanol (182 mg, 2.22 mmol) and triphenylphosphine (426 mg, 1.62 mmol) in THF (5 mL). This solution is cooled to 0° C. prior to slowly adding DIAD (0.32 mL, 1.62 mmol) over a 10 mins period. This mixture is stirred at RT for 2.5 h, then the solvent is removed in vacuo. The resulting mixture is purified by Combiflash eluting with hexane/EtOAc (10% to 100%). The first pure fractions coming from the column contain pure triazole X2 and the later fractions contain a mixture of triazole X2 along with another regioisomer (not characterized).

### Step 3

In a 50 mL-flask equipped with a 3-way adaptor is incorporated triazole X2 (25.0 mg, 0.094 mmol) and a spatula tip of 10% palladium on charcoal in MeOH (5 mL). The flask is purged with hydrogen (3×) and the reaction is kept under a hydrogen atmosphere (balloon) for 1.5 h. When the reaction is completed, the flask is purged with argon (3×) and the mixture is filtered over celite and rinsed thoroughly with MeOH. The filtrate is concentrated to afford R2k.

### Synthesis of 5-(1-hydroxy-1-methyl-ethyl)-thiophene 2-carboxylic acid (R2n)

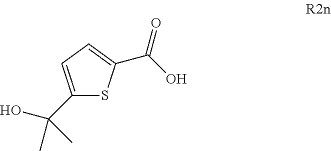

To a solution of 5-acetylthiophene-2-carboxylic acid (200 mg, 1.17 mmol) in THF (4 mL) at −20° C. is added a 1 M solution of methyl magnesium bromide in THF (2.94 mL, 2.94 mmol, 2.5 equiv). The reaction mixture is allowed to warm to RT slowly and is stirred at RT for 3 h. 10% citric acid (aq. solution) is added slowly, then the solution is concentrated. DCM is added followed by an extraction with DCM (3×). The combined organic phases are dried by passing trough a phase separator column to afford R2n.

¹H NMR (400 MHz, CDCl₃): δ 7.74 (d, 1H, J=3.9 Hz), 6.99 (d, 1H, J=3.9 Hz), 1.69 (s, 6H).

### Synthesis of Quinolines (Qa-Qh) from Table A2

Scheme 7 describes the general synthetic pathway to producing quinolines Qa-Qg.

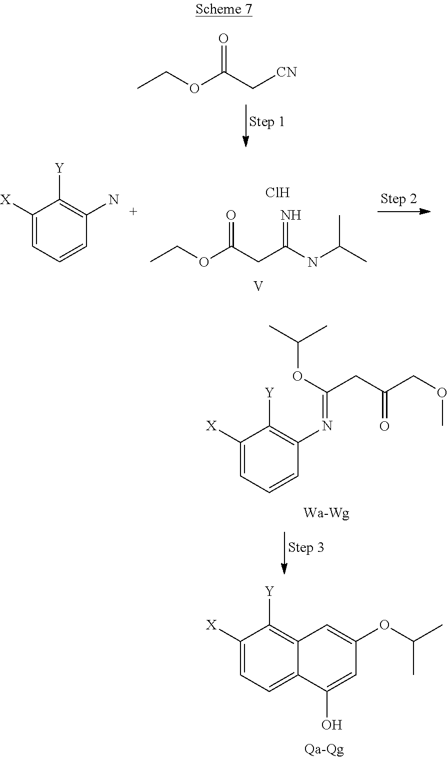

The synthesis of quinolines Qa to Qg requires the following anilines Ya-g:

| | |
|---|---|
| 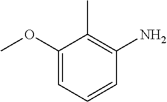 | Ya |
| 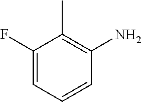 | Yb |
| 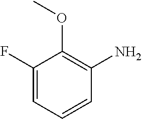 | Yc |
| 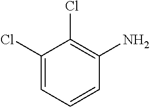 | Yd |
| 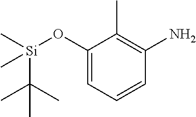 | Ye |
| 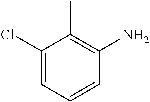 | Yf |
| 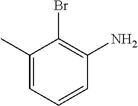 | Yg |

Anilines Ya, b, c, d, f and g are commercially available (Ya from TCI-US; Yb, Yd, Yf, Yg from Aldrich; Yc from Chontech)

Aniline Ye is prepared as follows:

### Synthesis of Aniline for Ye

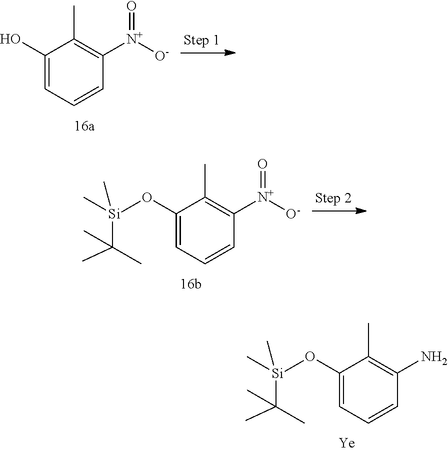

### Step 1

To a solution of the 2-methyl-3-nitrophenol 16a (5.00 g, 32.6 mmol) in DCM (60 mL) and DMF (15 mL) is added imidazole (4.45 g, 65.3 mmol) followed by tert-butyldimethylchlorosilane (6.40 g, 42.4 mmol) slowly. The solution is left to stir at RT for the night. DCM is removed under vacuum. The solution is taken up in EtOAc, washed with 0.1 N HCl, sat NaHCO₃ and brine (2×). The organic phase is dried over MgSO₄, filtered and concentrated. The crude material is purified by combiflash (80 g column starting with 1% EtOAc/hexane to 10% over 20 min) to afford 16b.

### Step 2

16b (5.30 g, 19.8 mmol) is dissolved in EtOH and the flask is purged with nitrogen. Palladium on carbon (400 mg) is added and the flask is evacuated and backfilled with hydrogen (3×). The reaction mixture is stirred at RT for 16 h. The flask is evacuated and backfilled with nitrogen (3×). The product is filtered through a celite pad, rinsed well with EtOAc and MeOH. The solution is evaporated to dryness to obtain an oil that is passed through a silica pad on a large fritted glass funnel with 50% EtOAc/hexane to obtain Ye.

Retention time (min)=3.5 min

¹H NMR (400 MHz, CDCl₃): 8, 6.88 (t, 1H, J=7.8 Hz), 6.34 (d, 1H, J=8.3 Hz), 6.29 (d, 1H, J=8.2 Hz), 3.60, (bs, 2H), 2.05 (s, 3H), 1.03 (s, 9H), 0.22 (s, 6H).

### Synthesis of Quinoline Qa

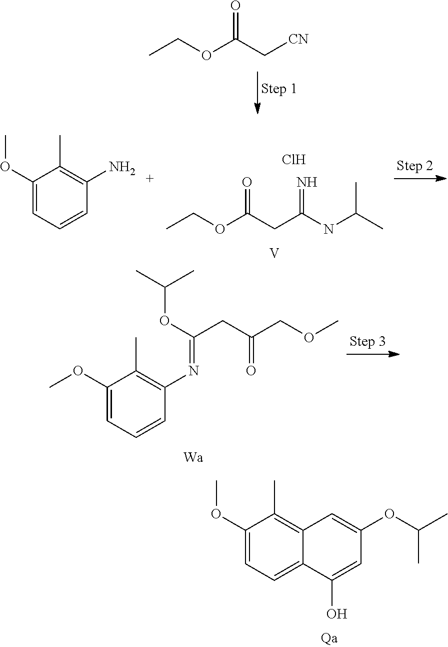

### Step 1: Synthesis of Imidate V

A solution of ethyl cyanoacetate (120 g, 1.06 mol) and isopropanol (70.1 g, 1.16 mol, 1.1 equiv) in anhydrous diethyl ether (1 L) is cooled to 0° C. This solution is purged with HCl gas for 45 mins and then the reaction mixture is warmed to ambient temperature and stirred for 19.5 h. The solvent is removed in vacuo, leaving a solid, which is triturated with hexanes, collected by suction filtration and dried in vacuo to yield imidate hydrochloride V. This intermediate is used in the next synthetic step without further purification.

### Step 2

In a 2 L round bottom flask, 3-methoxy-2-methyl aniline Ya (53.0 g, 386 mmol), intermediate V (81.0 g, 386 mmol, 1.00 equiv) and isopropanol (800 mL) are stirred at 40° C. for 3.5 h. The solvent is removed in vacuo, and the remaining residue is dissolved in EtOAc (1.5 L) and washed with brine (500 mL). The organic phase is dried over anhydrous sodium sulfate and concentrated to give Wa which is used in the next step without further purification.

### Step 3: Cyclization

In a 1 L round bottom flask, intermediate Wa (128 g) is dissolved in diphenyl ether (600 mL), and this mixture is quickly heated (heating mantle) to 230° C. The temperature is kept between 230° C. and 245° C. for 8 mins. The reaction mixture is cooled to RT, passed through a pad of silica gel (˜1 kg) and washed with hexanes to remove the diphenyl ether. The column is then eluted with a 20% to 80% EtOAc in hexanes to isolate Qa.

¹H NMR (400 MHz, DMSO-d₆) δ (ppm): 11.07 (s, 1H), 7.84 (d, 1H, J=9.2 Hz), 7.14 (d, 1H, J=9.2 Hz), 6.04 (s, 1H), 5.44 (S, J=6.3 Hz, 1H), 3.89 (s, 3H), 2.41 (s, 3H), 1.34 (d, 6H, J=6.3 Hz).

### Synthesis of Quinoline Qb

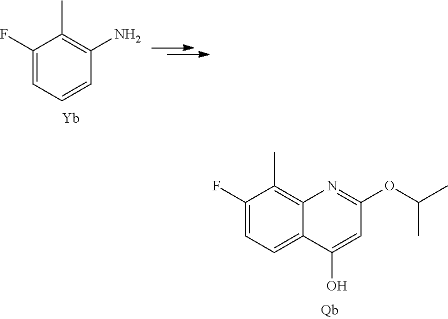

Quinoline Qb is prepared analogously to Qa by starting with aniline Yb.

¹H NMR (400 MHz, DMSO-d₆) δ (ppm): 11.44-11.25 (m, 1H), 7.85 (dd, 1H, J=9.3, 6.8 Hz), 7.13 (t, 1H, J=9.3 Hz), 6.16 (s, 1H), 5.44 (S, J=6.2 Hz, 1H), 2.44 (d, 3H, J=2.2 Hz), 1.33 (d, 6H, J=6.2 Hz).

### Synthesis of Quinoline Qc

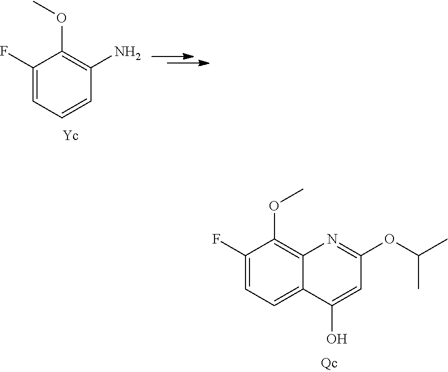

Quinoline Qc is prepared analogously to Qa by starting with aniline Yc.

### Synthesis of Quinoline Qd

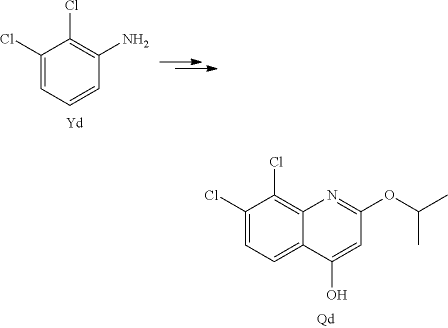

Quinoline Qd is prepared analogously to Qa by starting with aniline Yd.

¹H NMR (400 MHz, CDCl₃) δ (ppm): 9.06-8.98 (m, 1H), 8.16 (d, 1H, J=9.2 Hz), 7.38 (d, 1H, J=9.2 Hz), 5.97-5.87 (m, 1H), 4.84-4.70 (m, 1H), 1.46 (d, 6H, J=6.1 Hz).

### Synthesis of Quinoline Qe

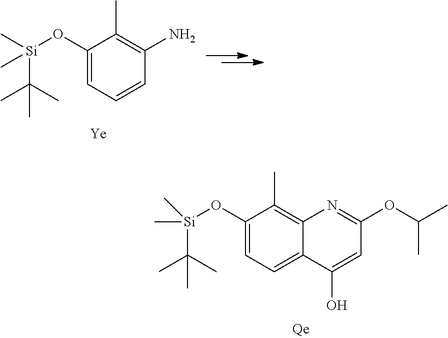

Quinoline Qe is prepared analogously to Qa by starting with aniline Ye.

¹H NMR (400 MHz, CDCl₃) δ (ppm): 8.06 (d, 1H, J=9.2 Hz), 7.61-7.52 (m, 1H), 6.78 (d, 1H, J=9.2 Hz), 5.69 (d, 1H, J=1.6 Hz), 4.69 (S, J=6.3 Hz, 1H), 2.27 (s, 3H), 1.42 (d, 6H, J=6.3 Hz), 1.04 (s, 9H), 0.26 (s, 6H).

### Synthesis of Quinoline Qf

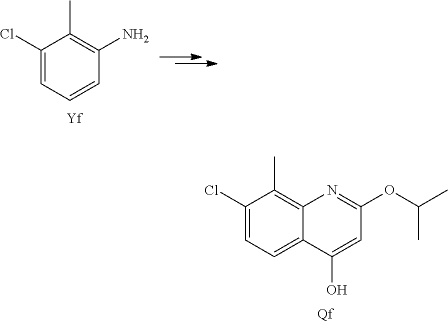

Quinoline Qf is prepared analogously to Qa by starting with aniline Yf.

¹H NMR (400 MHz, DMSO-d₆) δ (ppm): 11.43 (s, 1H), 7.82 (d, 1H, J=8.8), 7.32 (d, 1H, J=8.8 Hz), 6.21 (s, 1H), 5.44 (S, J=6.3 Hz, 1H), 2.96 (s, 3H), 1.35 (d, 6H, J=6.3 Hz).

### Synthesis of Quinoline Qa

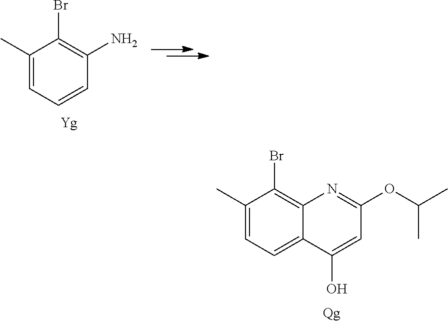

Quinoline Qg is prepared analogously to Qa by starting with aniline Yg.

¹H NMR (400 MHz, CDCl₃) δ (ppm): 8.35-8.19 (m, 1H), 8.16 (d, 1H, J=8.3 Hz), 7.19 (d, 1H, J=8.3 Hz), 5.82-5.74 (m, 1H), 4.78-4.67 (m, 1H), 2.55 (s, 3H), 1.45 (d, 6H, J=6.1 Hz).

### Synthesis of Quinoline Qh

Qh is prepared via an S_{N}Ar reaction with previously published intermediate 10a (WO 2009/014730).

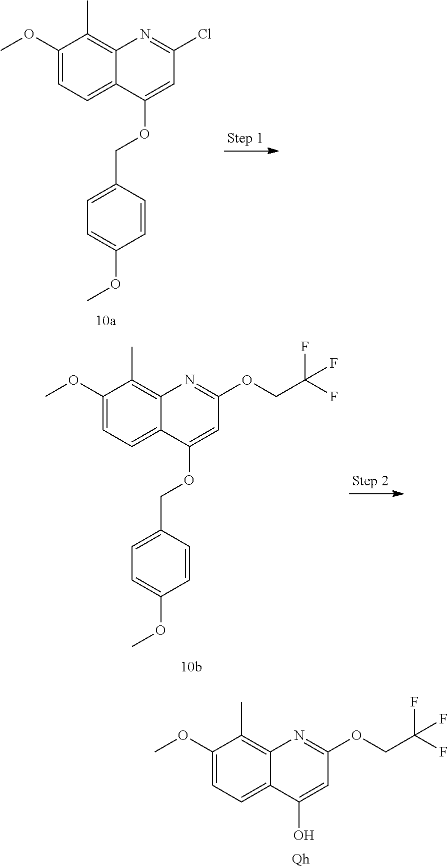

### Step 1

2,2,2-trifluoroethanol (2.182 g, 21.82 mmol) is added dropwise to NaH powder 60% (872 mg, 218.2 mmol, 10 equiv) as a suspension in DMF (5 mL) at 0° C. The mixture is stirred for 1 h at RT, then cooled to 0° C. 10a (750 mg, 2.18 mmol) is added in DMF (5 mL). The resulting mixture is stirred at 60° C. overnight. EtOAc is added and the organic phase is washed with NaHCO₃ (sat.), H₂O and brine; dried over MgSO₄, filtered and concentrated under reduced pressure. The crude material is purified by combiflash (silica gel 40 g, 2 to 10% EtOAc/hexanes) to give 10b.

### Step 2

10b (774 mg) is dissolved in CH₂Cl₂ (5 mL) and treated with TFA (3 mL) for 1 h. The reaction mixture is concentrated in vacuo to provide Qh.

¹H NMR (400 MHz, DMSO-d₆) δ (ppm): 11.48 (s, 1H), 7.91 (d, 1H, J=9.0 Hz), 7.23 (d, 1H, J=9.0 Hz), 6.21 (s, 1H), 5.10 (q, J=8.7 Hz, 2H), 3.91 (s, 3H), 2.43 (s, 3H).

### Synthesis of Macrocyclic Intermediate I from Schemes 3 and 4

The synthesis is performed as described in Scheme 8.

The synthesis of a close related analog (cyclopentyl carbamate instead of t-butyl carbamate) has been previously described in several patent applications and literature: (see for example: WO 2007030656; Tsantrizos et al., J. Organometallic Chem. 2006, 691, 5163-5171; Yee et al., J. Org. Chem. 2006, 71, 7133-7145). In particular, the synthesis of intermediate 1a and of 1c has been extensively described in the literature (see above references and references cited within).

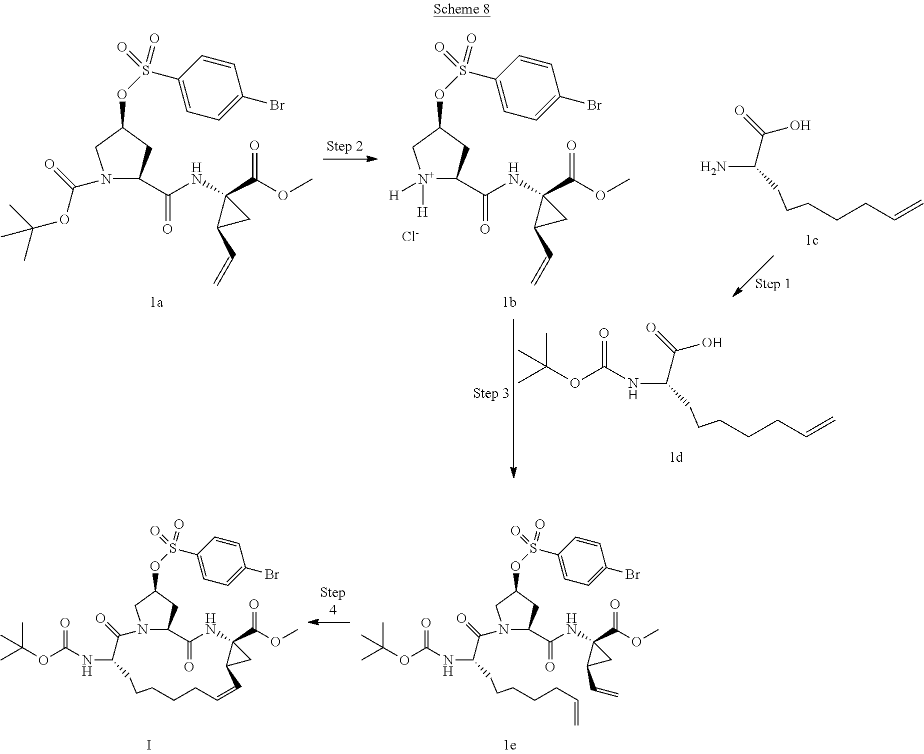

### Step 1

To a suspension of 1c (50.0 g, 292 mmol) in dioxane (300 mL) and water (200 mL) is added NaOH (12.8 g, 321 mmol) as a solution in water (150 mL). Boc₂O (76.6 g, 218 mmol) is dissolved in dioxane (50 mL) and added dropwise over a period of 30 mins. Addition of the reagent causes a suspension to form and there is a slight exotherm which is controlled with the use of a RT water bath. The reaction is left to stir overnight. The reaction mixture is transferred to a 2 L round-bottom flask using water (250 mL) for the transfer. The dioxane is evaporated at 40° C. Water is added to bring the volume to 1 L and 1 M NaOH (aq., ˜50 mL) is added to adjust the pH to −12. Any remaining solids are filtered and discarded. The aqueous solution is washed with a 50/50 mixture of t-BME/hexane (200 mL, 2×). The organic portions are discarded and the aqueous portion is transferred to a 2 L Erlenmeyer flask. t-BME (600 mL) is added and the mixture is cooled in an ice/water bath. 4 M HCl is added slowly until the pH is approximately 3. During the addition, a solid forms which causes the mixture to become an emulsion. The solids are filtered over a glass fiber filter disc and discarded. The filtrate is collected and the aqueous portion removed and extracted with t-BME (200 mL). The organic portions are combined and washed with 0.2 M KHSO₄ (200 mL, 2×), brine (200 mL), dried over Na₂SO₄, filtered and evaporated to give 1d.

### Step 2

Intermediate 1a (50.2 g, 87.5 mmol, 1.00 equiv) is suspended in EtOAc (225 mL). To the resulting slurry is added a 4 M HCl/dioxane solution (90 mL, 360 mol, 4.1 equiv) slowly from an addition funnel over about 30 mins, with vigorous stirring. The reaction is very slightly exothermic—the temperature of the reaction mixture rose from 20° C. to 25° C. (no cooling bath). At the end of the addition, all solids are dissolved. The reaction mixture is stirred for 3 h. The reaction mixture is concentrated in vacuo. The residual viscous liquid is diluted with EtOAc (500 mL) and re-concentrated. The residue is re-dissolved in EtOAc (500 mL), and then further diluted with Et₂O (500 mL). The solution is stored in the fridge (+5° C.) overnight. The precipitate which forms is collected using a sintered glass funnel and rinsed with EtOAc (500 mL, 2×) to give 1b.

### Step 3

The carbamate 1d (24.5 g, 90.3 mmol, 1.00 equiv) and HBTU (41.1 g, 108 mmol) are suspended in DCM (220 mL) and the suspension is stirred rapidly. DIPEA (15.7 mL, 90.4 mmol, 1.00 equiv) is added at ambient temperature and after 20 mins, a cloudy solution forms. A solution of 1b (47.9 g, 93.9 mmol, 1.04 equiv) in anhydrous DCM (330 mL) containing DIPEA (16.36 mL, 93.9 mmol 1.04 equiv) is then poured into the reaction. The resulting solution is allowed to stir for 16 h. The solvent is then evaporated yielding a syrup which is taken up in EtOAc (1.2 L) and washed with 0.05 N HCl (2×500 mL), sat Na₂CO₃ (800 mL) and brine (500 mL). The combined extracts are dried over NaSO₄ filtered and concentrated in vacuo. The material is purified by flash chromatography to yield the tripeptide 1e.

### Step 4

The tripeptide 1e (25.0 g, 34.4 mmol, 1.00 equiv) is dissolved in toluene (2.1 L). The reaction is heated to 80° C. While the mixture is heated, Ar is bubbled through the solution for 1 h. The catalyst (Hoveyda-Grubbs 2^{nd} generation catalyst from Aldrich, 0.3 g×4) is added in 4 equal portions, 30 mins apart. After complete addition, HPLC indicates that the ratio of product to starting material is about 35-40 to 1. The reaction is cooled to 50° C. and a solution of trihydroxymethyl phosphine (see below) is added and the mixture stirred at this temperature for 1 h. The mixture is cooled to RT and silica gel (21 g) is added and the mixture stirred a further 30 mins. The solids are filtered and washed with EtOAc, the filtrate and washings are combined, then washed with 0.5 M KHSO₄ (500 mL), sat NaHCO₃ (500 mL), water (500 mL) and brine (500 mL). The organic portion is dried over a combination of MgSO₄, silica gel and activated charcoal with stirring for 30 mins. The solids are filtered through a bed of celite and silica, and washed with small portions of EtOAc. The filtrate and washings are combined and evaporated. The residue is co-evaporated with t-BME (300 mL). t-BME (108 mL) is added, followed by the rapid addition of hexane (320 mL). A gummy solid forms which is stirred for about 48 h. A suspension forms which is further diluted with t-BME (40 mL) and the volume is adjusted to 800 mL with hexanes. The suspension is stirred for 30 mins. The solids are collected and washed with hexanes and air-dried to obtain macrocycle I.

### Trihydroxymethyl Phosphine Preparation

13 g of Tetrakishydroxymethylphosphoniumchloride (80% w/w H₂O) is dissolved in i-PrOH (21.30 mL) under a nitrogen atmosphere. 6.5 g of 45% w/w KOH in water (8 mL) is then added dropwise at RT. After stirring the suspension for 30 mins under nitrogen, the mixture is filtered and the solids washed with degassed i-PrOH (20 mL). The solids are discarded, and the filtrate and washings are combined and stored under nitrogen until used.

### Synthesis of Macrocyclic Intermediates via Scheme 3

### Synthesis of Macrocyclic Intermediates Aa and Ba

The synthesis of sulfonamides M and N are described in WO 2007009227.

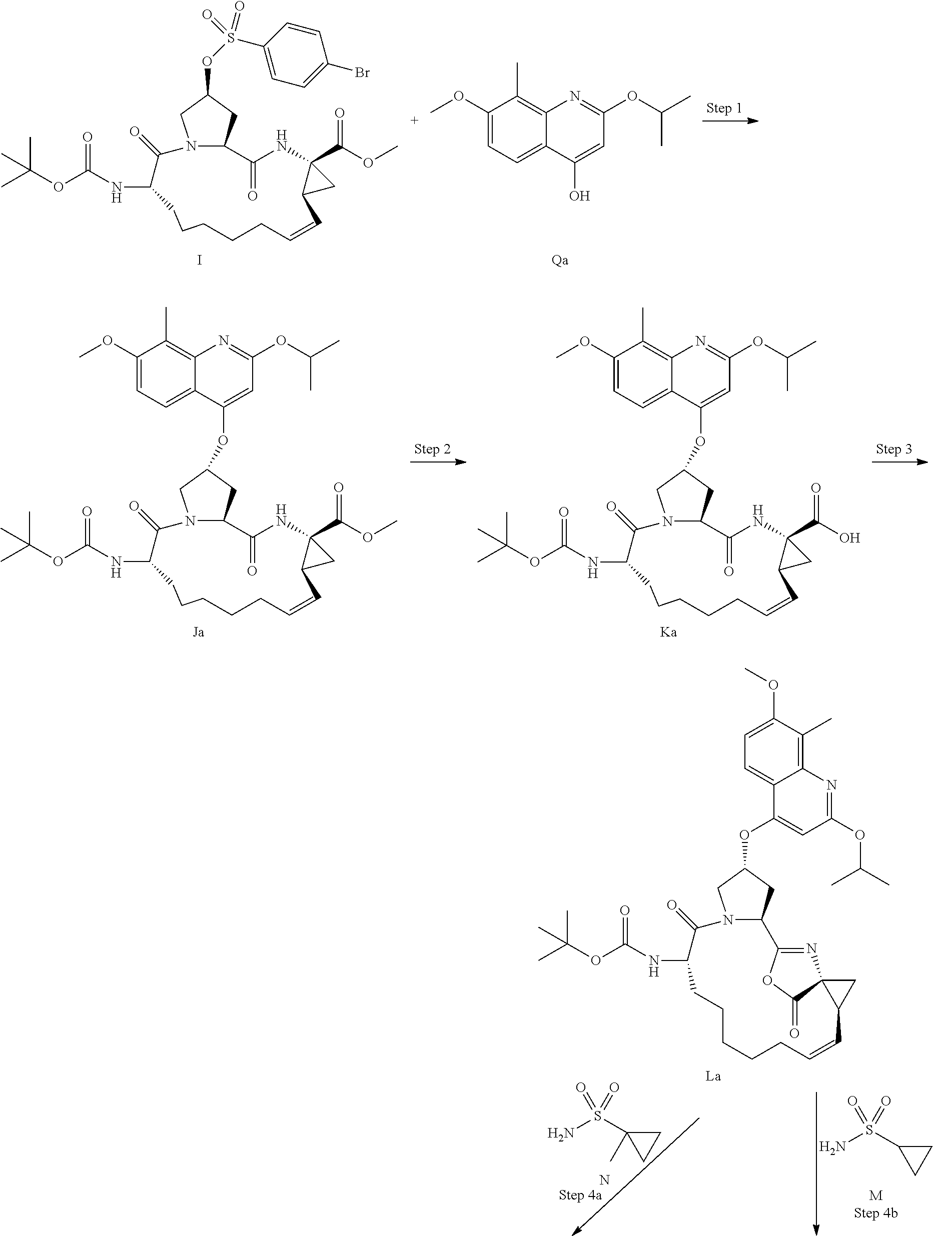

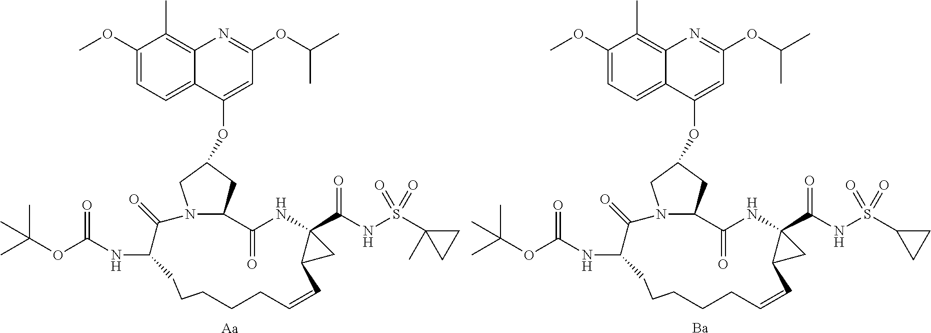

### Step 1: Brosylate Displacement

Macrocyclic Brosylate I (10.0 g, 14.3 mmol) and hydroxy quinoline Qa (3.90 g, 15.7 mmol) are dissolved in NMP (150 mL). Cs₂CO₃ (9.33 g, 28.6 mmol) is added and the mixture is heated to 70° C. for 8 h. The solution is cooled to RT and stirred an additional 8 h. The mixture is diluted with EtOAc and washed with H₂O (3×), NaHCO₃ (sat.) (2×), 1.0 N NaOH (1×), H₂O (2×) and brine (1×). The organics are dried over MgSO₄, filtered and concentrated in vacuo. The material is purified by flash chromatography using 30-40% EtOAc/hexanes as the eluent. The product containing fractions are combined and concentrated in vacuo to give Ja.

### Step 2: Hydrolysis

Ja (5.94 g, 8.38 mmol) is dissolved in THF/MeOH (2/1-120 mL) and 1 N NaOH (67 mL, 67 mmol) is added. The reaction mixture is stirred overnight at RT and then concentrated to dryness. The residue is then taken up in EtOAc/H₂O. The two phase mixture is acidified to pH ˜5 with 10% citric acid. The aqueous phase is extracted with EtOAc (3×) and the combined organics are washed with H₂O (3×), brine (1×), dried over MgSO₄, filtered and concentrated in vacuo to give the acid Ka which is used without further purification.

### Step 3: Azalactone Formation

The acid Ka (5.90 g, 8.38 mmol) is dissolved in DCM (55 mL). TEA (3.85 mL, 27.6 mmol) is added and the solution is cooled to 0° C. in an ice bath.

Isobutylchloroformate (1.63 mL, 12.5 mmol) is added dropwise and the mixture is stirred at 0° C. for 1 h and then allowed to warm slowly to RT and stirred overnight. The mixture is concentrated in vacuo and the residue taken up in THF/Et₂O (1:1, 100 mL). The mixture is filtered through celite to remove the salts. The mother liquor is concentrated to dryness to provide azalactone La which is used as such without further purification.

### Synthesis of Aa

### Step 4a: Acyl Sulfonamide Formation with Sulfonamide N

Sulfonamide N (2.40 g, 25.1 mmol) is dissolved in THF (100 mL) and cooled to −20° C. LiHMDS (1.0 N solution in THF, 21.8 mL, 21.8 mmol) is added all at once. The reaction is stirred at −20° C. for 5 mins and then allowed to warm to RT for 20 mins. The mixture is then recooled to −20° C. The azalactone La (5.67 g, 8.38 mmol) is dissolved in THF (40 mL) and added dropwise over 1 h to the sulfonamide anion solution. After the addition is complete, the mixture is allowed to warm to RT and the solution stirred overnight. Glacial HOAc (2.0 mL) is added and the reaction mixture is concentrated to dryness. The material is purified by flash chromatography using 25-55% EtOAc/hexanes as the eluent. The pure fractions are combined and concentrated in vacuo to provide Aa.

### Synthesis of Ba

### Step 4b: Synthesis of Ba—Acyl Sulfonamide Formation with Sulfonamide M

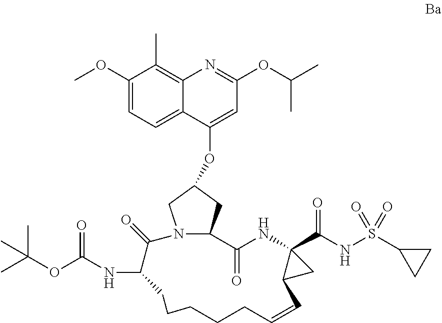

Intermediate Ba is prepared analogously to that of Aa by substituting sulfonamide M in place of sulfonamide N in step 4a of Scheme 3.

### Synthesis of Intermediate Ab

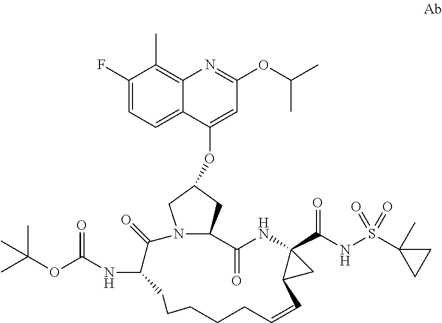

Intermediate Ab is prepared analogously to the procedure described for intermediate Aa with hydroxy quinoline Qb replacing Qa in step 1.

### Synthesis of Intermediate Ae

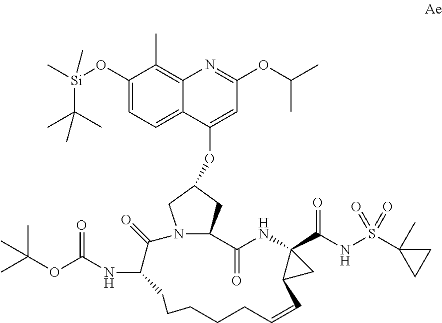

Intermediate Ae is prepared analogously to the procedure described for intermediate Aa with hydroxy quinoline Qe replacing Qa in step 1.

### Synthesis of Intermediate Af

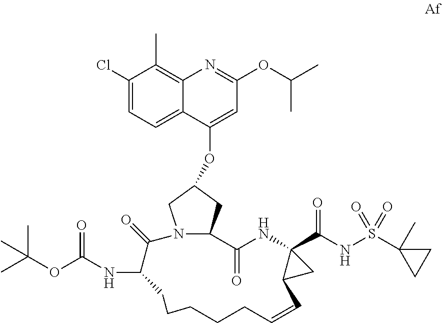

Intermediate Af is prepared analogously to the procedure described for intermediate Aa with hydroxy quinoline Qf replacing Qa in step 1.

### Synthesis of Intermediate Ah

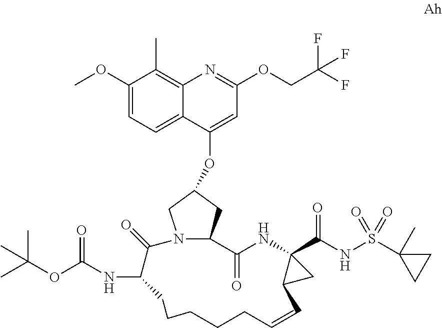

Intermediate Ah is prepared analogously to the procedure described for intermediate Aa with hydroxy quinoline Qh replacing Qa in step 1.

### Synthesis of Macrocyclic Intermediates via Scheme 4

### Synthesis of Intermediate Ac

Intermediate Ac is synthesized analogously to the general procedure described in Scheme 4.

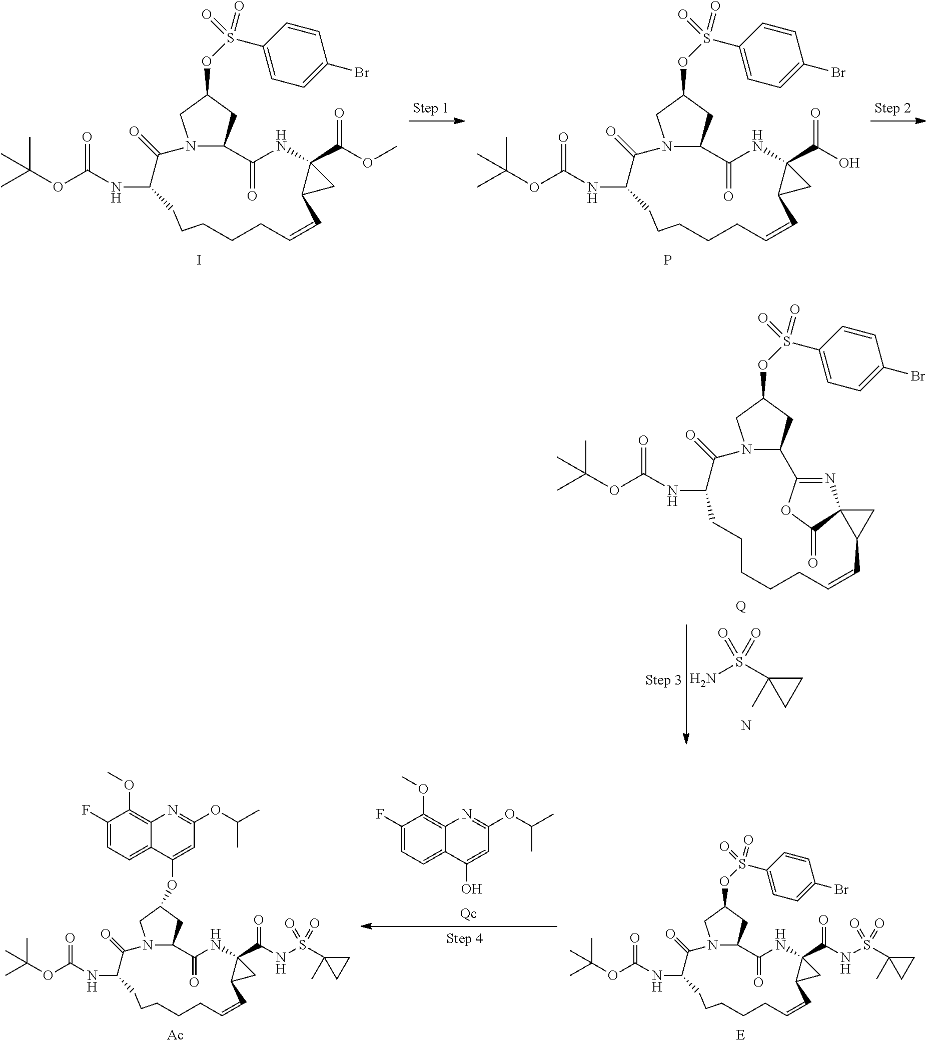

### Step 1: Hydrolysis

Intermediate I (3.0 g, 4.3 mmol) is dissolved in THF/MeOH (3/1, 28 mL) and 1.0 N NaOH (12.9 mL, 12.9 mmol) is added and the reaction is stirred overnight at RT. The reaction mixture is concentrated in vacuo, acidified with 10% citric acid to pH ˜6 and extracted with EtOAc (3×). The combined organic extracts are washed with H₂O (3×), brine (1×), dried over MgSO₄, filtered and concentrated in vacuo to yield carboxylic acid P.

### Step 2: Azalactone formation

Carboxylic acid P (11.5 g, 16.8 mmol) is dissolved in DCM (160 mL) and TEA (7.73 mL, 55.4 mmol) is added. The reaction mixture is cooled to 0° C. in an ice bath. Isobutylchloroformate (3.70 mL, 28.6 mmol) is added dropwise and the mixture is stirred at 0° C. for 1 h and then allowed to warm slowly to RT and stirred overnight. The mixture is concentrated in vacuo and purified by flash chromatography using 35-100% EtOAc/hexanes as the eluent, the pure fractions are combined and concentrated in vacuo to give azalactone Q.

### Step 3: Acylsulfonamide Formation

Sulfonamide N (2.96 g, 21.9 mmol) is dissolved in THF (80 mL) and cooled to −20° C. LiHMDS (1.0 N solution in THF, 18.8 mL, 18.8 mmol) is added all at once. The reaction is stirred at −20° C. for 5 mins and then allowed to warm to RT for 20 mins.

The mixture is then recooled to −20° C. The azalactone Q (5.66 g, 8.49 mmol) is dissolved in THF (40 mL) and added dropwise over 1 h to the sulfonamide anion solution. After the addition is complete, the mixture is allowed to warm to RT and the solution stirred overnight. Glacial HOAc (2.0 mL) is added and the reaction mixture is concentrated to dryness. The material is purified by flash chromatography using 30-85% EtOAc/hexanes as the eluent. The pure fractions are combined and concentrated in vacuo to provide E.

### Step 4: Brosylate Displacement with Hydroxy Quinoline (Qc)

Intermediate E (1.34 g, 1.67 mmol), and hydroxy quinoline Qc (400 mg, 1.59 mmol) are dissolved in NMP (10 mL), Cs₂CO₃ (2.08 g, 6.37 mmol) is added and the mixture is heated to 70° C. for 16 h. The solution is cooled to RT and the mixture is diluted with EtOAc and washed with H₂O (2×), 10% citric acid (1×) and brine (1×). The organics are dried over MgSO₄, filtered and concentrated in vacuo. The material is purified by flash chromatography using 20-60% EtOAc/hexanes as the eluent. The product containing fractions are combined and concentrated in vacuo to give Ac.

### Synthetic Examples of the Preparation of Compounds 1001-1029

### Synthesis of Compounds Prepared from Intermediate Aa

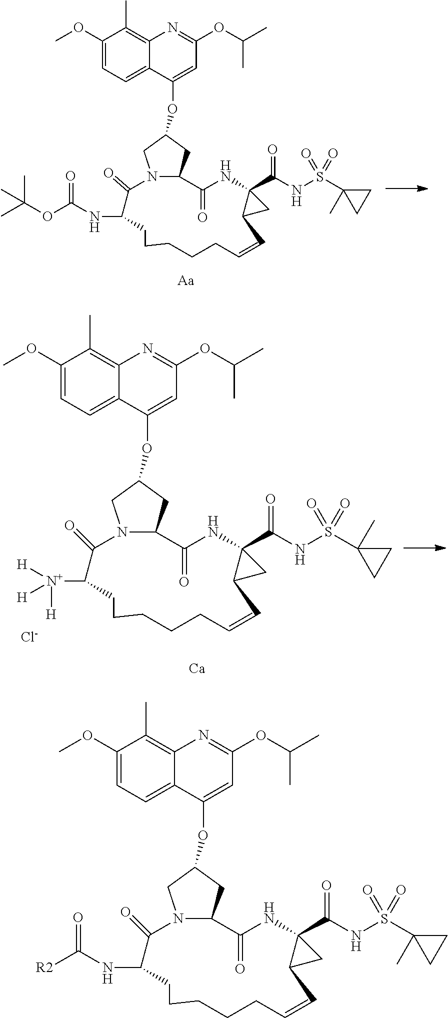

Boc protected macrocyclic amine Aa (1.50 g, 1.85 mmol) is charged in a vial with a 4 M solution of HCl in dioxane (37 mL, 148 mmol, 80 equiv). The solution is stirred at RT for 1 h, after which the solution is evaporated to dryness to provide compound Ca.

### Compound 1007

(2R,6S,12Z,13aS,14aR,16aS)-2-{[7-methoxy-8-methyl-2-(propan-2-yloxy)quinolin-4-yl]oxy}-N-[(1-methylcyclopropyl)sulfonyl]-6-{[(1-methyl-1H-pyrazol-3-yl)carbonyl]amino}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide

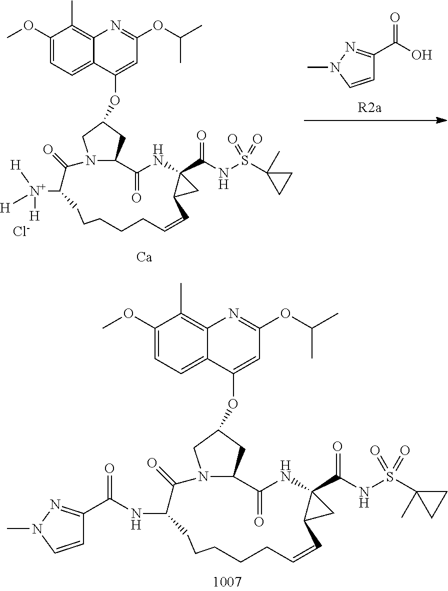

Acid R2a (6.6 mg, 0.052 mmol, 1.3 equiv) is dissolved in DMF (0.5 mL), then TEA (28 μL, 0.20 mmol, 5.0 equiv) is added followed by TBTU (15.4 mg, 0.048 mmol, 1.2 equiv). The solution is stirred for 15 mins, after which the amine hydrochloride Ca is added in DMF (0.5 mL) and this solution is stirred at RT for 16 h. Water (2 mL) is added to the solution, and then the organic layer is extracted with EtOAc (3×5 mL) and dried over MgSO₄. The solvent is evaporated and the residue is purified on prep HPLC (MeCN:H₂O, 0.1% TFA). The pure fractions are combined, concentrated, frozen and lyophilized to provide compound 1007.

FIA M.S. (electrospray): 820.3 (M+H)⁺

Retention time (min): 5.7 min

¹H NMR (400 MHz, DMSO-d₆): δ 10.83 (s, 1H), 8.93 (s, 1H), 7.83 (d, 1H, J=8.8 Hz), 7.79 (d, 1H, J=7 Hz), 7.77 (d, 1H, J=2.1 Hz), 7.08 (d, 1H, J=9 Hz), 6.60 (d, 1H, J=2.3 Hz), 6.37 (s, 1H), 5.66-5.58 (m, 1H), 5.62 (S, 1H, J=6.2 Hz), 5.47-5.44 (m, 1H), 5.07 (dd, 1H, J=9.5, 9.2 Hz), 4.64-4.54 (m, 1H) 4.52 (d, 1H, J=11.6 Hz), 4.40 (dd, 1H, J=9.7, 7.1 Hz), 4.02 (dd, 1H, J=11.8, 3.5 Hz), 3.89 (s, 3H), 3.88 (s, 3H), 2.66-2.57 (m, 1H), 2.51 (s, 3H), 2.43 (s, 3H), 2.38-2.29 (m, 2H), 2.02-1.90 (m, 1H), 1.88-1.77 (m, 1H), 1.58 (dd, 1H, J=8.2, 5.1 Hz), 1.52 (dd, 1H, J=9.3, 5.2 Hz), 1.45-1.35 (m, 12H), 1.34-1.20 (m, 4H), 0.93-0.84 (m, 2H).

### Compound 1001

(2R,6S,12Z,13aS,14aR,16aS)-6-({[1-(2,2-difluoroethyl)-1H-pyrazol-3-yl]carbonyl}amino)-2-{[7-methoxy-8-methyl-2-(propan-2-yloxy)quinolin-4-yl]oxy}-N-[(1-methylcyclopropyl)sulfonyl]-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide

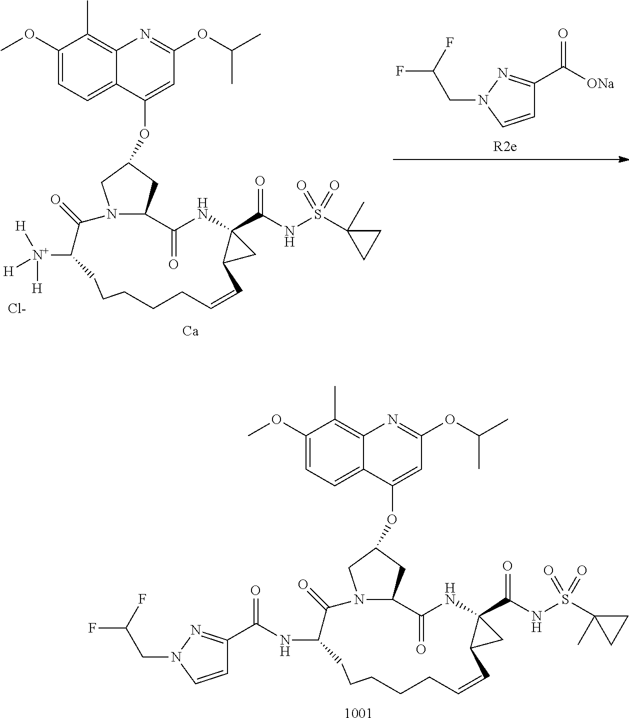

The crude acid R2e (27.0 mg, 0.14 mmol) is dissolved in DMF (1 mL) along with diisopropylethyl amine (71 μL, 0.41 mmol) and HATU (51.8 mg, 0.14 mmol). After stirring for 15 mins, the macrocyclic amine hydrochloride salt Ca (50 mg, 0.068 mmol) is transferred as a 1 mL DMF solution and stirred at RT for 16 h. The crude mixture is filtered with a Millex filter and purified directly by prep HPLC (MeOH, ammonium formate). The appropriate fractions are combined, frozen and lyophilized to give compound 1001.

FIA M.S. (electrospray): 870.4 (M+H)⁺, 868.5 (M−H)⁻

Retention time (min): 5.9 min

¹H NMR (400 MHz, DMSO-d₆): δ 10.82 (bs, 1H), 8.91 (bs, 1H), 7.88 (bs, 1H), 7.86 (d, 1H, J=2.4 Hz), 7.82 (d, 1H, J=9.0 Hz), 7.07 (d, 1H, J=9.0 Hz), 6.69 (d, 1H, J=2.4 Hz), 6.42 (tt, 1H, ¹J_{1-F}=54.8 Hz, J=3.6 Hz), 6.35 (s, 1H), 5.65-5.56 (m, 1H), 5.48 (S, 1H, J=6.3 Hz), 5.44 (bs, 1H), 5.11-5.03 (m, 1H), 4.70 (td, 2H, J=14.9, 3.6 Hz), 4.64-4.57 (m, 1H), 4.56-4.48 (m, 1H), 4.40 (t, 1H, J=8.2 Hz), 4.07-4.00 (m, 1H), 3.87 (s, 3H), 2.64-2.54 (m, 2H), 2.43 (s, 3H), 2.38-2.29 (m, 2H), 2.01-1.91 (m, 1H), 1.87-1.78 (m, 1H), 1.59-1.48 (m, 4H), 1.46-1.35 (m, 5H), 1.39 (d, 3H, J=6.3 Hz), 1.38 (s, 3H), 1.37 (d, 3H, J=6.2 Hz), 1.32-1.21 (m, 2H), 0.90-0.84 (m, 2H).

### Compound 1002

(2R,6S,12Z,13aS,14aR,16aS)-6-({[1-(2-methoxyethyl)-1H-pyrazol-3-yl]carbonyl}amino)-2-{[7-methoxy-8-methyl-2-(propan-2-yloxy)quinolin-4-yl]oxy}-N-[(1-methylcyclopropyl)sulfonyl]-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide

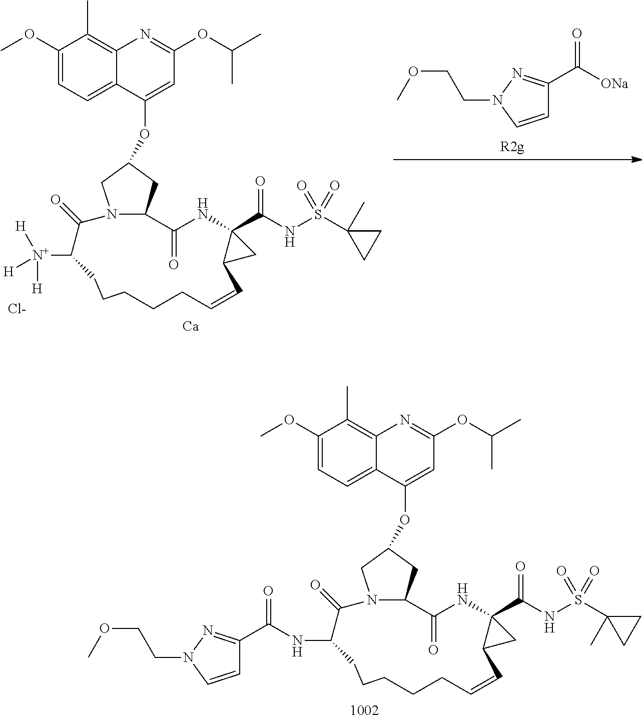

### Compound 1002 is synthesized analogously to compound 1001 by using the macrocyclic amine hydrochloride salt Ca (53.9 mg, 0.072 mmol) in the presence of the crude intermediate R2g (27.7 mg, 0.14 mmol).

UPLC M.S. (electrospray): 864.4 (M+H)⁺, 862.5 (M−H)⁻

Retention time (min): 5.7 min

¹H NMR (400 MHz, DMSO-d₆): δ 10.83 (bs, 1H), 8.92 (bs, 1H), 7.83 (d, 1H, J=9.0 Hz), 7.80 (bs, 1H), 7.78 (d, 1H, J=2.4 Hz), 7.08 (d, 1H, J=9.0 Hz), 6.60 (d, 1H, J=2.4 Hz), 6.35 (s, 1H), 5.65-5.54 (m, 1H), 5.48 (S, 1H, J=6.1 Hz), 5.44 (s, 1H), 5.16-5.02 (m, 1H), 4.69-4.47 (m, 2H), 4.41 (t, 1H, J=8.2 Hz), 4.31 (t, 2H, J=5.3 Hz), 4.09-4.00 (m, 1H), 3.87 (s, 3H), 3.70 (t, 2H, J=5.3 Hz), 3.22 (s, 3H), 2.94-2.87 (m, 1H), 2.63-2.53 (m, 2H), 2.43 (s, 3H), 2.38-2.29 (m, 2H), 2.00-1.80 (m, 2H), 1.61-1.49 (m, 3H), 1.46-1.39 (m, 4H), 1.39 (d, 3H, J=6.2 Hz), 1.38 (s, 3H), 1.38 (d, 3H, J=6.0 Hz), 1.32-1.19 (m, 4H), 0.91-0.80 (m, 2H).

### Compound 1004

(2R,6S,12Z,13aS,14aR,16aS)-6-({[1-(2-fluoroethyl)-1H-pyrazol-3-yl]carbonyl}amino)-2-{[7-methoxy-8-methyl-2-(propan-2-yloxy)quinolin-4-yl]oxy}-N-[(1-methylcyclopropyl)sulfonyl]-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide

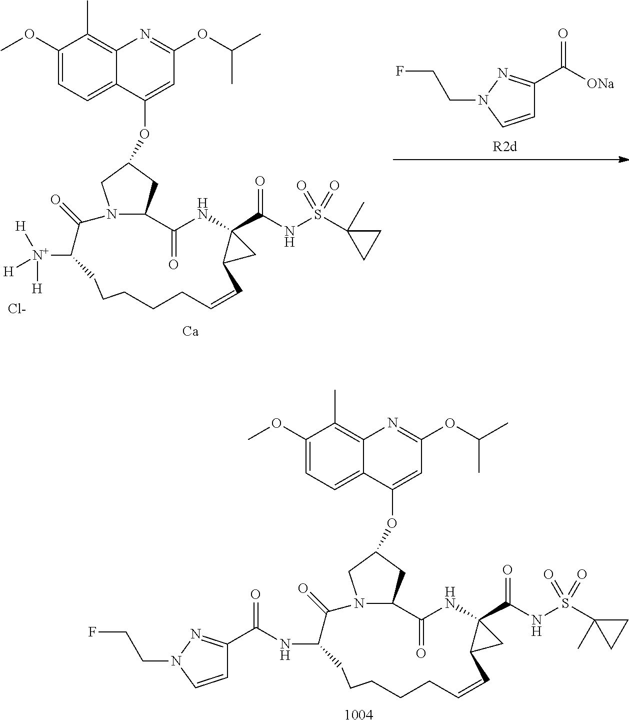

### Compound 1004 is synthesized analogously to compound 1001 by using the macrocyclic amine hydrochloride salt Ca (50 mg, 0.067 mmol) in the presence of the crude intermediate R2d (24.1 mg, 0.13 mmol).

FIA M.S. (electrospray): 852.4 (M+H)⁺, 850.5 (M−H)

Retention time (min): 5.8 min

¹H NMR (400 MHz, DMSO-d₆): δ 10.83 (bs, 1H), 8.91 (bs, 1H), 7.85 (bs, 1H), 7.85 (d, 1H, J=2.4 Hz), 7.82 (d, 1H, J=9.0 Hz), 7.07 (d, 1H, J=9.0 Hz), 6.64 (d, 1H, J=2.4 Hz), 6.35 (s, 1H), 5.64-5.54 (m, 1H), 5.48 (S, 1H, J=6.2 Hz), 5.44 (bs, 1H), 5.11-5.04 (m, 1H), 4.80 (dt, 2H, ¹J_{1-F}=47.4 Hz, J=4.7 Hz), 4.67-4.56 (m, 1H), 4.49 (dt, 2H, J=27.8, 4.7 Hz), 4.45-4.38 (m, 2H), 4.09-3.99 (m, 1H), 3.87 (s, 3H), 2.62-2.54 (m, 2H), 2.43 (s, 3H), 2.39-2.29 (m, 2H), 2.00-1.91 (m, 1H), 1.88-1.75 (m, 1H), 1.59-1.49 (m, 3H), 1.46-1.39 (m, 4H), 1.39 (d, 3H, J=6.2 Hz), 1.38 (s, 3H), 1.37 (d, 3H, J=6.2 Hz), 1.30-1.20 (m, 4H), 0.91-0.84 (m, 2H).

### Compound 1005

(2R,6S,12Z,13aS,14aR,16aS)-2-{[7-methoxy-8-methyl-2-(propan-2-yloxy)quinolin-4-yl]oxy}-N-[(1-methylcyclopropyl)sulfonyl]-5,16-dioxo-6-({[1-(2,2,2-trifluoroethyl)-1H-pyrazol-3-yl]carbonyl}amino)-1,2,3,6,7,8,9,10,11,13a,14,15,16,16a-tetradecahydrocyclopropa[e]pyrrolo[1,2-a][1,4]diazacyclopentadecine-14a(5H)-carboxamide

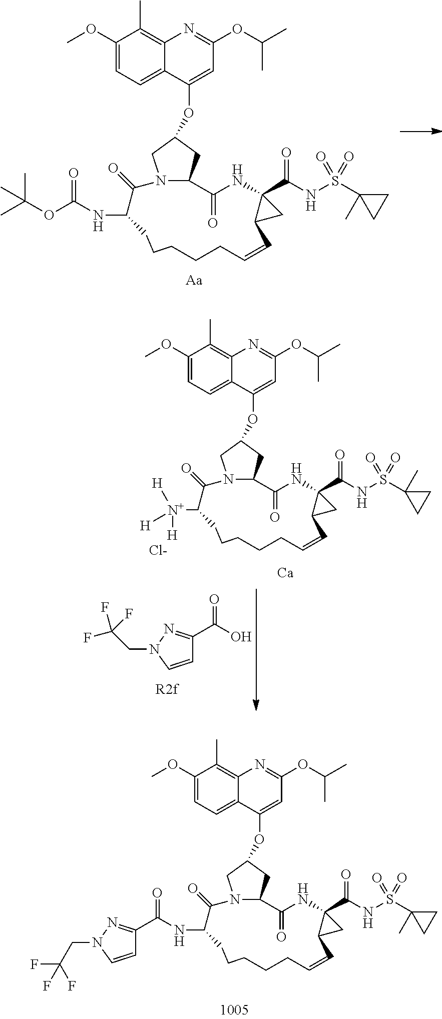

Boc protected amine Aa (40 mg, 0.049 mmol) is charged in a vial, then a 4 M solution of HCl in dioxane (1 mL, 4 mmol) is added. The solution is stirred at RT for 2 h, after which a precipitate forms. The solution is evaporated to dryness which affords the amine hydrochloride Ca.

Acid R2f (12 mg, 0.064 mmol, 1.3 equiv) is dissolved in DMF (0.5 mL), then TEA (34 μL, 0.25 mmol, 5.0 equiv) is added followed by TBTU (22 mg, 0.059 mmol, 1.2 equiv). The solution is stirred for 15 mins, after which the amine hydrochloride Ca is added in DMF (0.5 mL). The resulting solution is stirred at RT for 16 h. Water (2 mL) is added and the organic layer is extracted with EtOAc (3×5 mL). The solvent is evaporated and purification is performed by prep HPLC (MeCN:H₂O, 0.1% TFA). The pure fractions are combined, concentrated, frozen and lyophilized to provide compound 1005.

FIA M.S. (electrospray): 888.5 (M+H)⁺

Retention time (min): 6.1 min

¹H NMR (400 MHz, DMSO-d₆): δ 10.80 (s, 1H), 8.93 (s, 1H), 7.95-7.90 (m, 2H), 7.84 (d, 1H, J=8.9 Hz), 7.07 (d, 1H, J=8.9 Hz), 6.75 (d, 1H, J=2.7 Hz), 6.37 (s, 1H), 5.67-5.59 (m, 1H), 5.55-5.44 (m, 2H), 5.27-5.18 (q, 2H, J=9.2 Hz), 5.07 (dd, 1H, J=9.5, 9.0 Hz), 4.65-4.59 (m, 1H) 4.53 (d, 1H, J=11.5 Hz), 4.43 (dd, 1H, J=9.6, 7.1 Hz), 4.03 (dd, 1H, J=11.8, 3.7 Hz), 3.88 (s, 3H), 2.66-2.57 (m, 1H), 2.44 (s, 3H), 2.40-2.29 (m, 2H), 2.04-1.92 (m, 1H), 1.89-1.78 (m, 1H), 1.63-1.49, (m, 3H), 1.47-1.36 (m, 14H), 1.34-1.20 (m, 4H), 0.93-0.84 (m, 2H).

### Compound 1006

(2R,6S,12Z,13aS,14aR,16aS)-2-{[7-methoxy-8-methyl-2-(propan-2-yloxy)quinolin-4-yl]oxy}-N-[(1-methylcyclopropyl)sulfonyl]-6-{[(2-methyl-2H-1,2,3-triazol-4-yl)carbonyl]amino}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide

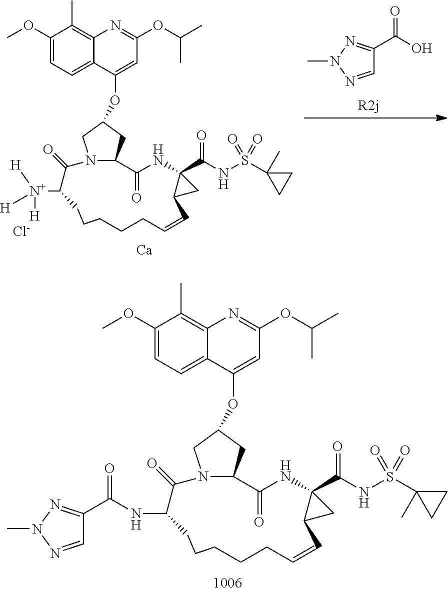

Acid R2j (17.7 mg, 0.140 mmol, 1.2 equiv) is dissolved in DMF (0.5 mL), then TEA (81 μL, 0.58 mmol, 5.0 equiv) is added followed by TBTU (52.9 mg, 0.140 mmol, 1.2 equiv). The solution is stirred for 15 mins, after which the amine hydrochloride Ca (87 mg, 0.116 mmol) is added in DMF (1 mL). The resulting solution is stirred at RT for 16 h. Water (2 mL) is added and the organic layer is extracted with EtOAc (3×5 mL). The solvent is evaporated and the residue is purified on CombiFlash (25-70% EtOAc/Hexane). The pure fractions are combined, concentrated, frozen and lyophilized to provide compound 1006.

FIA M.S. (electrospray): 821.5 (M+H)⁺; 819.3 (M−H)⁻

Retention time (min): 5.6 min

¹H NMR (400 MHz, DMSO-d₆): δ 10.80 (s, 1H), 8.92 (s, 1H), 8.07 (s, 1H), 8.35 (d, 1H, J=6.7 Hz), 7.85 (d, 1H, J=8.8 Hz), 7.10 (d, 1H, J=8.8 Hz), 6.35 (s, 1H), 5.67-5.59 (m 1H), 5.53-5.43 (m, 2H), 5.10-5.03 (m, 1H), 4.62-4.52 (m, 2H), 4.45-4.39 (m, 1H), 4.20 (s, 3H), 4.06-3.98 (m, 1H), 3.89 (s, 3H), 2.69-2.66 (m, 1H), 2.44 (s, 3H), 2.40-2.31 (m, 2H), 2.06-1.94 (m, 1H), 1.89-1.76 (m, 1H), 1.62-1.49 (m, 2H), 1.47-1.35 (m, 14H), 1.34-1.20 (m, 5H), 0.94-0.83 (m, 2H).

### Compound 1010

(2R,6S,12Z,13aS,14aR,16aS)-6-({[2-(2,2-difluoroethyl)-2H-1,2,3-triazol-4-yl]carbonyl}amino)-2-{[7-methoxy-8-methyl-2-(propan-2-yloxy)quinolin-4-yl]oxy}-N-[(1-methylcyclopropyl)sulfonyl]-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide

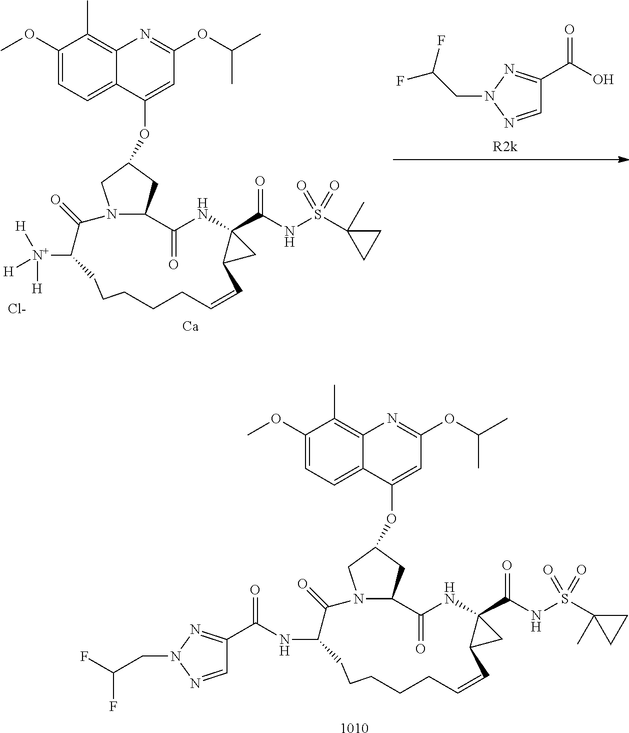

The crude macrocylic intermediate Ca (50.0 mg, 0.067 mmol) is dissolved in DMF (1 mL) along with crude carboxylic acid intermediate R2k (16.0 mg, 0.090 mmol), TEA (47 μL, 0.33 mmol) and TBTU (32.2 mg, 0.10 mmol). When the reaction is completed, the crude mixture is filtered with a Millex filter and purified directly by prep HPLC (MeOH, pH 10). The appropriate fractions are combined, frozen and lyophilized to give compound 1010.

FIA M.S. (electrospray): 871.4 (M+H)⁺, 869.5 (M−H)⁻

Retention time (min): 6.0 min

¹H NMR (400 MHz, DMSO-d₆): δ 10.82 (bs, 1H), 8.91 (bs, 1H), 8.54 (bs, 1H), 8.23 (bs, 1H), 7.85 (d, 1H, J=9.0 Hz), 7.09 (d, 1H, J=9.0 Hz), 6.51 (tt, 1H, ¹J_{1-F}=54.1 Hz, J=3.5 Hz), 6.35 (s, 1H), 5.62 (s, 1H), 5.49 (S, 1H, J=6.1 Hz), 5.44 (s, 1H), 5.08 (s, 1H), 5.04 (td, 2H, J=14.9, 3.3 Hz), 4.57 (bs, 1H), 4.40 (t, 1H, J=8.0 Hz), 4.06-3.98 (m, 1H), 3.88 (s, 3H), 2.65-2.54 (m, 2H), 2.43 (s, 3H), 2.39-2.29 (m, 2H), 2.04-1.93 (m, 1H), 1.87-1.71 (m, 1H), 1.60-1.47 (m, 4H), 1.49-1.37 (m, 5H), 1.39 (d, 3H, J=6.1 Hz), 1.38 (s, 3H), 1.38 (d, 3H, J=6.1 Hz), 1.32-1.20 (m, 3H), 0.91-0.83 (m, 2H).

### Compound 1017

(2R,6S,12Z,13aS,14aR,16aS)-6-[({1-[2-(dimethylamino)-2-oxoethyl]-1H-pyrazol-3-yl}carbonyl)amino]-2-{[7-methoxy-8-methyl-2-(propan-2-yloxy)quinolin-4-yl]oxy}-N-[(1-methylcyclopropyl)sulfonyl]-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide

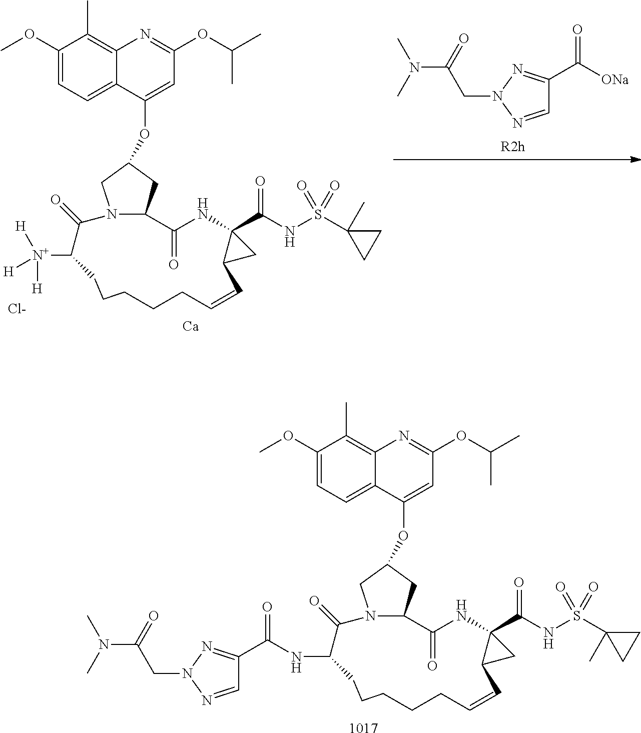

### Compound 1017 is synthesized analogously to compound 1001 by using the macrocyclic amine hydrochloride salt Ca (50 mg, 0.067 mmol) in the presence of the crude intermediate R2h (24.1 mg, 0.13 mmol).

FIA M.S. (electrospray): 891.5 (M+H)⁺

Retention time (min): 5.4 min

¹H NMR (400 MHz, DMSO-d₆): δ 10.83 (s, 1H), 8.89 (s, 1H), 7.81 (d, 1H, J=9.0 Hz), 7.74-7.69 (m, 1H), 7.71 (d, 1H, J=2.3 Hz), 7.09 (d, 1H, J=9.0 Hz), 6.63 (d, 1H, J=2.3 Hz), 6.35 (s, 1H), 5.63-5.53 (m, 1H), 5.48 (p, 1H, J=6.0 Hz), 5.44 (m, 1H), 5.20 (s, 2H), 5.14-5.05 (s, 1H), 4.72-4.60 (m, 1H), 4.55-4.38 (m, 2H), 4.10-4.00 (m, 1H), 3.87 (s, 3H), 3.02 (s, 3H), 2.85 (s, 3H), 2.62-2.54 (m, 1H), 2.42 (s, 3H), 2.40-2.26 (m, 2H), 1.99-1.89 (m, 1H), 1.85-1.77 (m, 1H), 1.57-1.51 (m, 3H), 1.45-1.19 (m, 12H), 1.38 (d, 3H, J=6.0 Hz), 1.37 (d, 3H, J=6.0 Hz), 0.91-0.78 (m, 2H).

### Compound 1019

(2R,6S,12Z,13aS,14aR,16aS)-2-{[7-methoxy-8-methyl-2-(propan-2-yloxy)quinolin-4-yl]oxy}-6-[({1-[2-(methylamino)-2-oxoethyl]-1H-pyrazol-3-yl}carbonyl)amino]-N-[(1-methylcyclopropyl)sulfonyl]-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide

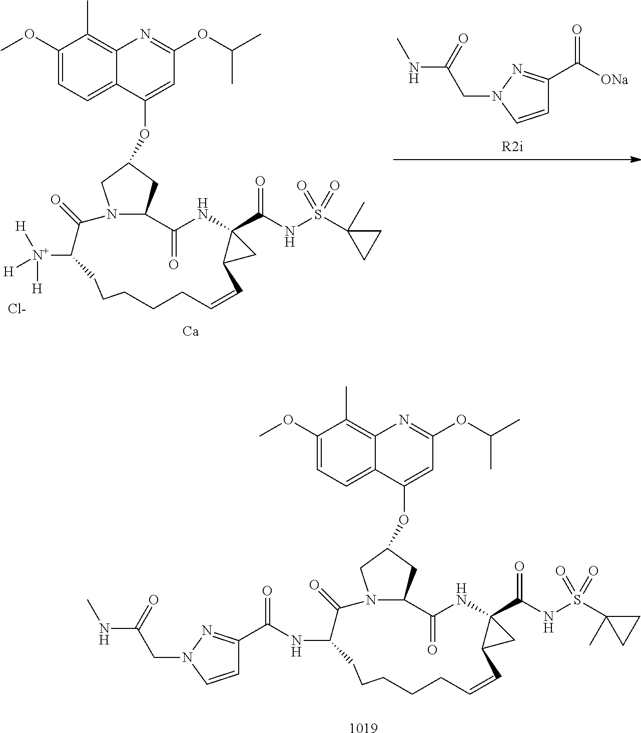

### Compound 1019 is synthesized analogously to compound 1001 by using the macrocyclic amine hydrochloride salt Ca (55 mg, 0.068 mmol) in the presence of the crude intermediate R2I (24.1 mg, 0.13 mmol).

FIA M.S. (electrospray): 877.3 (M+H)⁺

Retention time (min): 5.2 min

¹H NMR (400 MHz, DMSO-d₆): δ 10.80 (b, 1H), 8.91 (b, 1H), 8.09-8.05 (m, 1H), 7.81-7.78 (m, 3H), 7.14-7.01 (m, 1H), 7.08 (d, 1H, J=9.4 Hz), 6.63 (d, 1H, J=2.4 Hz), 6.35 (s, 1H), 5.57-5.39 (m, 3H), 5.17-5.11 (b, 1H), 4.89-4.82 (m, 1H), 4.85 (s, 2H), 4.73-4.60 (b, 1H), 4.50-4.38 (m, 2H), 3.87 (s, 3H), 2.62 (d, 3H, J=4.7 Hz), 2.60-2.54 (m, 1H), 2.42 (s, 3H), 2.39-2.30 (m, 2H), 1.99-1.90 (m, 2H), 1.58-1.51 (m, 3H), 1.44-1.15 (m, 12H), 1.38 (d, 3H, J=6.0 Hz), 1.37 (d, 3H, J=6.0 Hz), 0.89-0.78 (b, 1H).

### Compound 1020

(2R,6S,12Z,13aS,14aR,16aS)-6-({[5-(2-hydroxypropan-2-yl)thiophen-2-yl]carbonyl}amino)-2-{[7-methoxy-8-methyl-2-(propan-2-yloxy)quinolin-4-yl]oxy}-N-[(1-methylcyclopropyl)sulfonyl]-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide

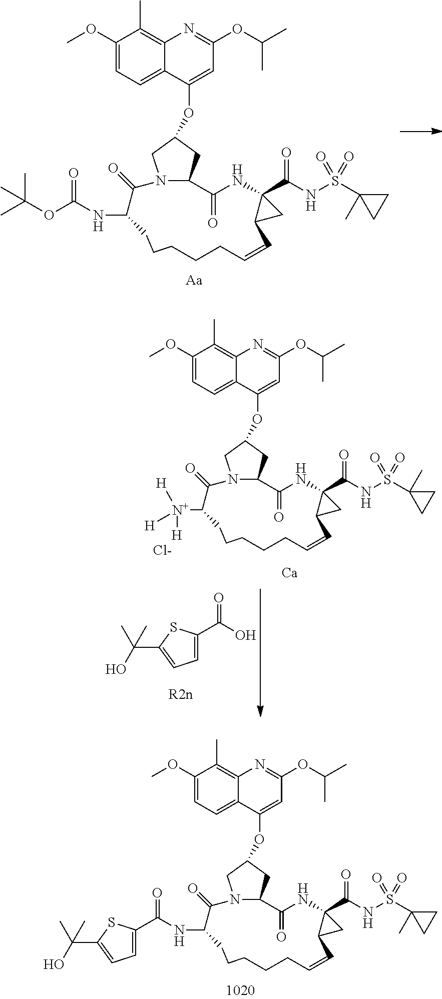

Boc protected amine macrocycle Aa (82 mg, 0.101 mmol) is charged in a flask then a 4 M solution of HCl in dioxane (0.75 mL, 3.03 mmol) is added. The solution is stirred at RT for 1.0 h, after which a precipitate forms. The solution is then evaporated to dryness which affords the amine hydrochloride Ca. Acid R2n (21.6 mg, 0.116 mmol) is dissolved in DCM (2 mL), then TEA (56 μL, 0.404 mmol) is added followed by TBTU (37 mg, 0.116 mmol). The solution is stirred for 15 mins, after then added to the amine hydrochloride Ca in solution in DCM (1 mL). The resulting solution is stirred at RT for 16 h and then concentrated. The residual is dissolved in DMSO, filtered through a Millex filter and purified by prep HPLC (ammonium formate/MeOH). The pure fractions are combined, concentrated, redissolved in MeCN and water, frozen and lyophilized to provide compound 1020.

FIA M.S. (electrospray): 880.4 (M+H)⁺

Retention time (min): 5.9 min

¹H NMR (400 MHz, DMSO-d₆): δ 10.84 (bs, 1H), 8.82 (bs, 1H), 8.60 (d, 1H, J=5.5 Hz), 8.04 (d, 1H, J=9.0 Hz), 7.71 (d, 1H, J=3.5 Hz), 7.15 (d, 1H, J=9.4 Hz), 6.92 (d, 1H, J=3.5 Hz), 6.35 (s, 1H), 5.64-5.56 (m 2H), 5.49 (S, 1H J=6.3 Hz), 5.44-5.40 (m, 1H), 5.17-5.03 (m, 1H), 4.77 (d, 1H, J=10.1 Hz), 4.55-4.46 (m, 1H), 4.32 (dd, 1H, J=7.4, 7.1 Hz), 3.96 (d, 1H, J=8.6 Hz), 3.89 (s, 3H), 2.68-2.55 (m, 2H), 2.44 (s, 3H), 2.36-2.28 (m, 2H), 2.07-1.96 (m, 1H), 1.84-1.68 (m, 1H), 1.57-1.35 (m, 23H), 1.31-1.20 (m, 3H), 0.91-0.78 (m, 2H).

### Synthesis of Compounds Prepared from Intermediate Ba

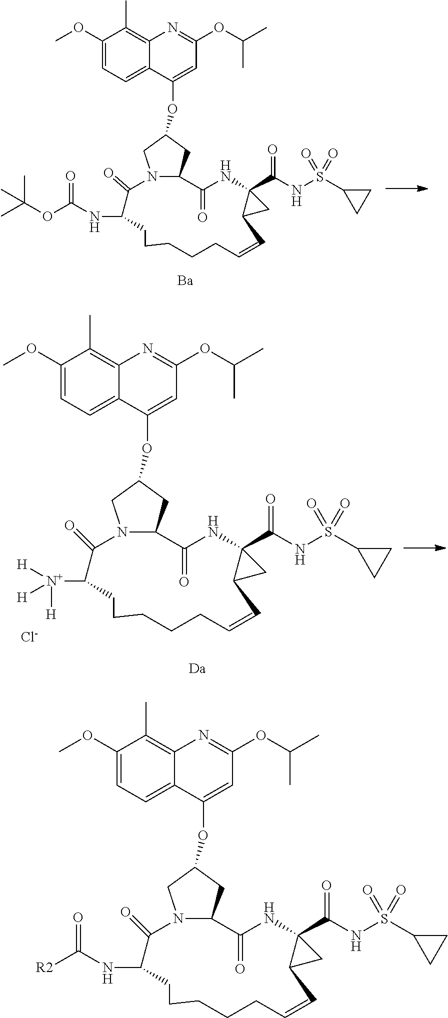

Boc protected macrocyclic amine Ba (400 mg, 0.50 mmole) is charged in a vial with a 4 M solution of HCl in dioxane (10 mL, 40 mmol, 80 equiv). The solution is stirred at RT for 2 h, after which the solution is evaporated to dryness to provide compound Da.

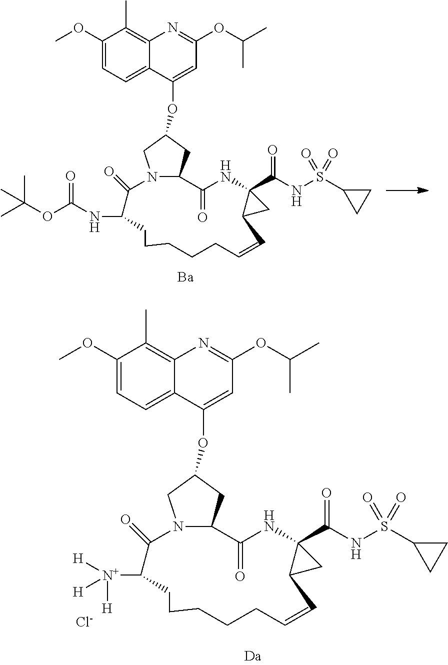

### Compound 1009

(2R,6S,12Z,13aS,14aR,16aS)-N-(cyclopropylsulfonyl)-2-{[7-methoxy-8-methyl-2-(propan-2-yloxy)quinolin-4-yl]oxy}-6-({[5-(methoxymethyl)thiophen-2-yl]carbonyl}amino)-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide

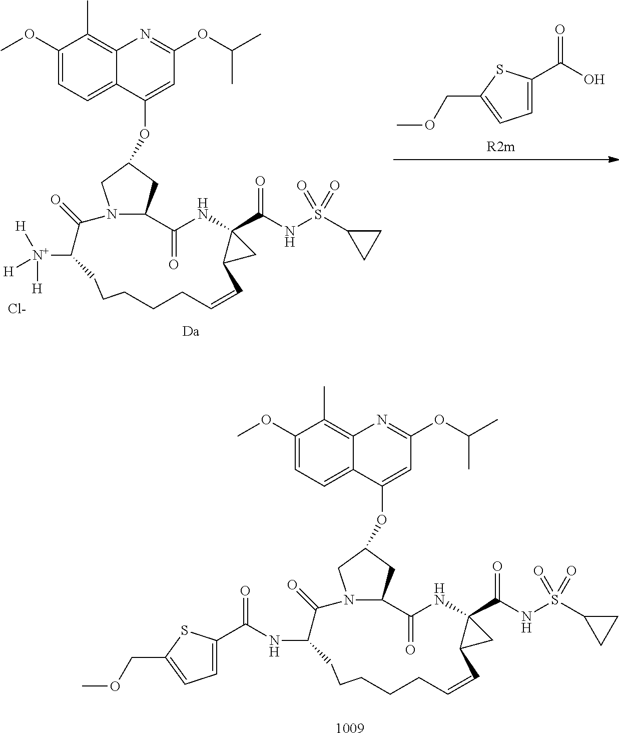

Macrocyclic amine hydrochloride Da (36.8 mg, 0.050 mmol) is dissolved in DMF (1.0 mL), then diisopropylethylamine (44 μL; 0.251 mmol), the acid R2m (9.7 mg; 0.056 mmol) and HATU (23.3 mg; 0.061 mmol) are added. The reaction is stirred at RT overnight. The resulting solution is filtered through a Millex filter and purified by prep HPLC (ammonium bicarbonate/MeOH). The pure fractions are combined, concentrated, frozen and lyophilized to provide compound 1009.

FIA M.S. (electrospray): 850.5 (M−H)−, 852.5 (M+H)⁺

Retention time (min): 5.8 min

¹H NMR (400 MHz, DMSO-d₆): δ 11.02 (s, 1H), 8.74 (s, 1H), 8.69 (d, 1H, J=5.5 Hz), 7.96 (d, 1H, J=9 Hz), 7.76 (d, 1H, J=3.7 Hz), 7.07 (d, 1H, J=9.2 Hz), 7.04 (d, 1H, J=3.9 Hz), 6.34 (s, 1H), 5.65-5.55 (m 1H), 5.51-5.48 (m, 1H), 5.42 (bs, 1H), 5.18-5.08 (m, 1H), 4.71 (d, 1H, J=11.1 Hz), 4.58 (s, 2H), 4.53-4.45 (m, 1H), 4.33-4.29 (m, 1H), 3.98-3.94 (m, 1H), 3.88 (s, 3H), 3.29 (s, 3H), 2.95-2.85 (m, 1H), 2.64-2.56 (m, 2H), 2.43 (s, 3H), 2.42-2.28 (m, 2H), 2.04-1.95 (m, 1H), 1.83-1.68 (m, 1H), 1.58-1.40 (m, 7H), 1.38 (d, 3H, J=5.1 Hz), 1.38 (d, 3H, J=5.1 Hz), 1.33-1.20 (m, 2H), 1.11-0.97 (m, 4H).

Compound 1011 (2R,6S,12Z,13aS,14aR,16aS)-N-(cyclopropylsulfonyl)-6-({[1-(2,2-difluoroethyl)-1H-pyrazol-3-yl]carbonyl}amino)-2-{[7-methoxy-8-methyl-2-(propan-2-yloxy)quinolin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide

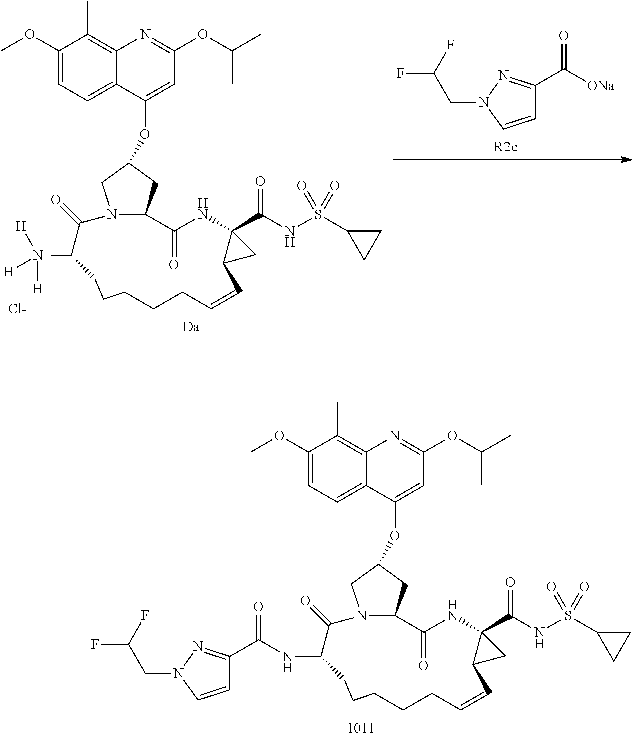

The crude intermediate R2e (27.0 mg, 0.14 mmol) is dissolved in DMF (1 mL) along with diisopropylethyl amine (71 μL, 0.41 mmol) and HATU (51.8 mg, 0.14 mmol). After stirring for 15 mins, the macrocyclic amine hydrochloride salt Da (50 mg, 0.068 mmol) is transferred as a 1 mL DMF solution and stirred at RT for 16 h. The crude mixture is filtered with a Millex filter and purified directly by prep HPLC (MeOH, ammonium formate). The appropriate fractions are combined, frozen and lyophilized to give compound 1011.

FIA M.S. (electrospray): 856.4 (M+H)⁺, 854.5 (M−H)⁻

Retention time (min): 5.8 min

¹H NMR (400 MHz, DMSO-d₆): δ 11.05 (bs, 1H), 8.79 (bs, 1H), 7.89 (bs, 1H), 7.86 (d, 1H, J=2.4 Hz), 7.82 (d, 1H, J=9.0 Hz), 7.07 (d, 1H, J=9.0 Hz), 6.69 (d, 1H, J=2.4 Hz), 6.42 (tt, 1H, ¹J_{1-F}=55.0 Hz, J=3.5 Hz), 6.35 (s, 1H), 5.66-5.57 (m, 1H), 5.49 (S, 1H, J=5.9 Hz), 5.46-5.41 (m, 1H), 5.17-5.11 (m, 1H), 4.70 (td, 2H, J=15.0, 3.5 Hz), 4.63-4.55 (m, 1H), 4.52 (d, 1H, J=11.4 Hz), 4.39-4.32 (m, 1H), 4.04-3.98 (m, 1H), 3.87 (s, 3H), 2.94-2.87 (m, 1H), 2.64-2.54 (m, 4H), 2.43 (s, 3H), 2.39-2.29 (m, 2H), 1.99-1.89 (m, 1H), 1.85-1.74 (m, 1H), 1.60-1.51 (m, 3H), 1.46-1.36 (m, 4H), 1.39 (d, 3H, J=5.9 Hz), 1.38 (d, 3H, J=5.9 Hz), 1.29-1.18 (m, 2H), 1.09-0.99 (m, 4H).

### Compound 1012

(2R,6S,12Z,13aS,14aR,16aS)-N-(cyclopropylsulfonyl)-2-{[7-methoxy-8-methyl-2-(propan-2-yloxy)quinolin-4-yl]oxy}-5,16-dioxo-6-({[1-(2,2,2-trifluoroethyl)-1H-pyrazol-3-yl]carbonyl}amino)-1,2,3,6,7,8,9,10,11,13a,14,15,16,16a-tetradecahydrocyclopropa[e]pyrrolo[1,2-a][1,4]diazacyclopentadecine-14a(5H)-carboxamide

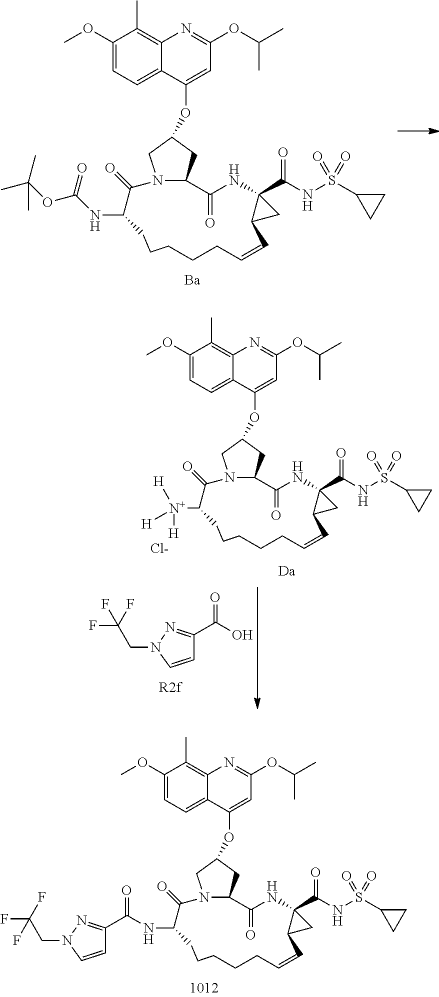

Boc Macrocycle Ba (200 mg, 0.25 mmol) is dissolved in DCM (1 mL) and a commercial solution of 4N HCl in dioxane (2 mL, 8.0 mmol, 32 equiv) is added. This mixture is stirred at RT for 60 mins, and then concentrated under reduced pressure to afford a residue corresponding to the unprotected amine Da.

The crude intermediate Da is dissolved in DMF (5 mL) along with TEA (0.18 mL, 1.25 mmol, 5 equiv), 1-(2,2,2-trifluoro-ethyl)-1H-pyrazole-3-carboxylic acid R2f (58 mg, 0.30 mmol, 1.2 equiv) and TBTU (121 mg, 0.38 mmol, 1.5 equiv). Upon stirring at RT for 3 h, H₂O and EtOAc are added. The layers are separated and the aqueous phase is extracted with EtOAc (2×). The organics are combined and dried by passing through an IST separator cartridge, and then concentrated. The mixture is purified by Combiflash (12 g column, eluent: Hex/EtOAc, 20% to 100% gradient). A second purification by Combiflash is performed (12 g column, eluent: Hex/EtOAc, 60% isocratic). The pure fractions are combined and concentrated. The product is then re-dissolved in acetonitrile and filtered through a Millex filter, frozen and lyophilized to provide compound 1012.

UPLC-MS (electrospray): 874.4 (M+H), 872.5 (M−H)

Retention time (min): 6.0 min

¹H NMR (400 MHz, DMSO-d₆): δ 11.05 (s, 1H), 8.79 (s, 1H), 7.97 (d, J=7.0 Hz, 1H), 7.93 (d, J=2.4 Hz, 1H), 7.84 (d, J=9.0 Hz, 1H), 7.07 (d, J=9.0 Hz, 1H), 6.76 (d, J=2.4 Hz, 1H), 6.36 (s, 1H), 5.65-5.58 (m, 1H), 5.49 (S, J=6.0 Hz, 1H), 5.44 (bs, 1H), 5.23 (q, J=9.0 Hz, 1H), 5.14 (dd, J=9.4, 9.8 Hz, 1H), 4.62-4.59 (m, 1H), 4.53 (d, J=11.6 Hz, 1H), 4.36 (dd, J=7.0, 9.8 Hz, 1H), 4.00 (dd, J=3.3, 11.6 Hz, 1H), 3.87 (s, 3H), 2.95-2.88 (m, 1H), 2.63-2.55 (m, 2H), 2.43 (s, 3H), 2.38-2.28 (m, 2H), 2.00-1.91 (m, 1H), 1.83-1.73 (m, 1H), 1.59-1.52 (m, 3H), 1.48-1.22 (m, 7H), 1.39 (d, J=5.9 Hz, 3H), 1.38 (d, J=5.9 Hz, 3H), 1.11-0.98 (m, 4H).

### Compound 1014

(2R,6S,12Z,13aS,14aR,16aS)-N-(cyclopropylsulfonyl)-6-({[5-(2-hydroxypropan-2-yl)thiophen-2-yl]carbonyl}amino)-2-{[7-methoxy-8-methyl-2-(propan-2-yloxy)quinolin-4-yl]oxy}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide

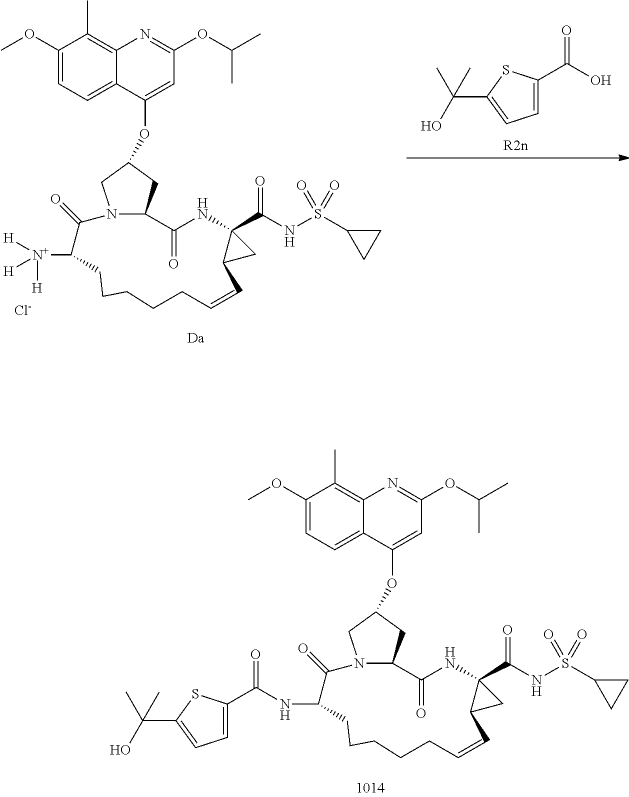

Acid R2n (18 mg, 0.098 mmol, 1.2 equiv) is dissolved in DMF (1 mL), then TEA (45 mL, 0.326 mmol, 5.0 equiv) is added followed by TBTU (30 mg, 0.094 mmol, 1.15 equiv). The solution is stirred for 15 mins, after which the amine hydrochloride Da (60 mg, 0.082 mmol) is added in DMF (1 mL). The resulting solution is stirred at RT for 16 h. Water (2 mL) is added and then the organic layer is extracted with EtOAc (3×5 mL). The solvent is evaporated and the residue is purified on prep HPLC (MeCN:H₂O, 0.1% TFA). The pure fractions are combined, concentrated, frozen and lyophilized to provide compound 1014.

FIA M.S. (electrospray): 866.4 (M+H)⁺; 864.5 (M−H)⁻

Retention time (min): 5.7 min

¹H NMR (400 MHz, DMSO-d₆): δ 11.01 (s, 1H), 8.72 (s, 1H), 8.58 (d, 1H, J=5.9 Hz), 8.04 (d, 1H, J=9.2 Hz), 7.69 (d, 1H, J=3.9 Hz), 7.15 (d, 1H, J=9.3 Hz), 6.91 (d, 1H, J=3.9 Hz), 6.34 (s, 1H), 5.66-5.57 (m 1H), 5.49 (S, 1H J=6.2 Hz), 5.44-5.40 (m, 1H), 5.12 (dd, 1H, J=9.9, 8.9 Hz), 4.77 (d, 1H, J=11.4 Hz), 4.51-4.44 (m, 1H), 4.28 (dd, 1H, J=10.0, 6.8 Hz), 3.93 (dd, 1H, J=11.3, 3.1 Hz), 3.89 (s, 3H), 2.93-2.86 (m, 1H), 2.71-2.55 (m, 2H), 2.44 (s, 3H), 2.40-2.26 (m, 2H), 2.07-1.95 (m, 1H), 1.79-1.68 (m, 1H), 1.33-1.19 (m, 2H), 1.59-1.35 (m, 19H), 1.12-0.97 (m, 4H).

### Compound 1018

(2R,6S,12Z,13aS,14aR,16aS)-N-(cyclopropylsulfonyl)-2-{[7-methoxy-8-methyl-2-(propan-2-yloxy)quinolin-4-yl]oxy}-6-[({1-[2-(methylamino)-2-oxoethyl]-1H-pyrazol-3-yl}carbonyl)amino]-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide

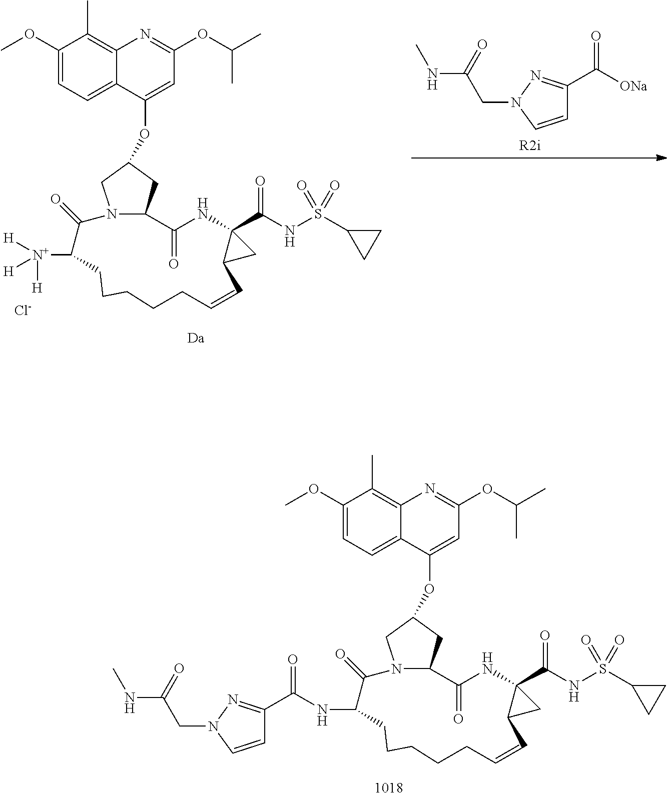

### Compound 1018 is made analogously to the procedure used for the preparation of compound 1014 using 50 mg (0.063 mmol) of Da and 25 mg of R2i.

FIA M.S. (electrospray): 863.3 (M+H)⁺

Retention time (min): 5.1 min

¹H NMR (400 MHz, DMSO-d₆): δ 11.04 (s, 1H), 8.77 (s, 1H), 8.09-8.05 (m, 1H), 7.83-7.80 (m, 1H), 7.78 (d, 1H, J=2.3 Hz), 7.72-7.60 (m, 1H), 7.08 (d, 1H, J=9.4 Hz), 6.63 (d, 1H, J=2.3 Hz), 6.35 (s, 1H), 5.65-5.56 (m, 1H), 5.49 (p, 1H, J=6.0 Hz), 5.44 (m, 1H), 5.17-5.11 (m, 1H), 4.85 (s, 2H), 4.62-4.50 (m, 2H), 4.39-4.30 (m, 1H), 4.04-3.98 (m, 1H), 3.88 (s, 3H), 2.94-2.88 (m, 1H), 2.62 (d, 3H, J=4.7 Hz), 2.60-2.54 (m, 1H), 2.43 (s, 3H), 2.39-2.30 (m, 2H), 1.99-1.90 (m, 1H), 1.83-1.73 (m, 1H), 1.59-1.52 (m, 3H), 1.46-1.20 (m, 7H), 1.38 (d, 3H, J=6.0 Hz), 1.37 (d, 3H, J=6.0 Hz), 1.10-0.98 (m, 4H).

### Synthesis of Compounds Prepared from Intermediate Ab

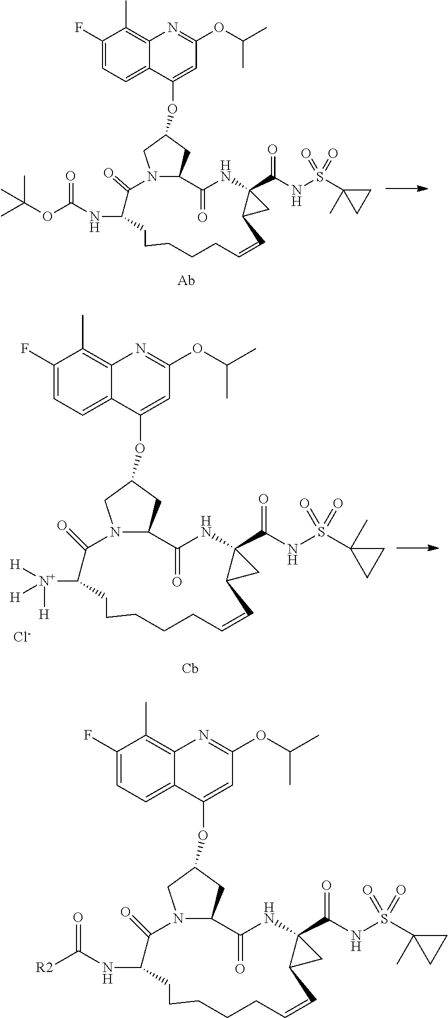

### Compound 1008

(2R,6S,12Z,13aS,14aR,16aS)-2-{[7-fluoro-8-methyl-2-(propan-2-yloxy)quinolin-4-yl]oxy}-N-[(1-methylcyclopropyl)sulfonyl]-6-{[(1-methyl-1H-pyrazol-3-yl)carbonyl]amino}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide

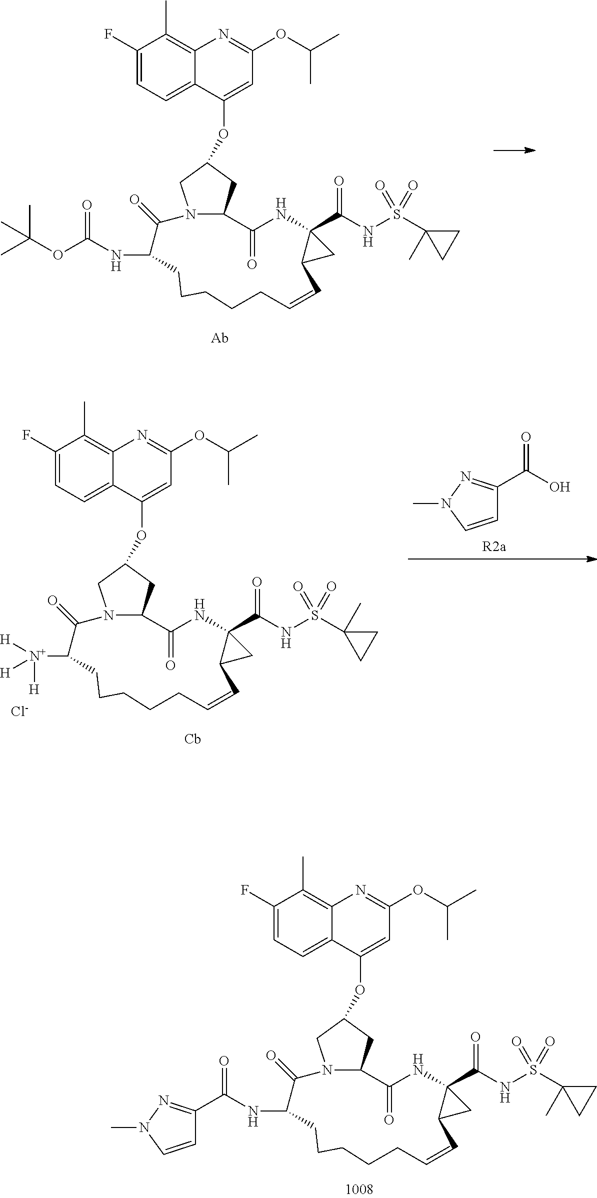

Boc protected amine macrocycle Ab (74 mg, 0.093 mmol) is charged in a vial, then a 4 M solution of HCl in dioxane (3 mL, 12 mmol) is added. The solution is stirred at RT for 1 h, after which a precipitate forms. The solution is then evaporated to dryness to afford Cb.

Acid R2a (14 mg, 0.111 mmol, 1.2 equiv) is dissolved in DMF (1 mL), then TEA (51.6 μL, 0.370 mmol) is added followed by TBTU (34.2 mg, 0.111 mmol, 1.2 equiv). The solution is stirred for 15 mins, after which the amine hydrochloride Cb is added in DMF (1 mL) and the solution is stirred at RT for 16 h. The resulting solution is filtered through a Millex filter and purified by prep HPLC (Sunfire column, 0.1% TFA) The pure fractions are combined, concentrated, frozen and lyophilized to provide compound 1008.

FIA M.S. (electrospray): 808.4 (M+H)⁺

Retention time (min): 6.7 min

¹H NMR (400 MHz, DMSO-d₆): δ 10.83 (s, 1H), 8.92 (s, 1H), 7.90 (dd, 1H, J=8.6, 6.6 Hz), 7.79 (d, 1H, J=6.3 Hz), 7.75 (d, 1H, J=1.9 Hz), 7.12 (t, 1H, J=9.4 Hz), (d, 1H, J=2.4 Hz), 6.52 (s, 1H), 5.65-5.58 (m 1H), 5.53-5.47 (m, 2H), 5.08-5.04 (m, 1H), 4.57-4.54 (m, 2H), 4.42-4.38 (m, 1H), 4.02 (m, 1H), 3.88 (s, 3H), 2.64-2.58 (m, 2H), 2.48 (s, 3H), 2.36-2.32 (m, 2H), 1.99-1.91 (m, 1H), 1.88-1.73 (bm, 1H), 1.59-1.49 (m, 3H), 1.41 (s, 3H), 1.39 (d, J=5.5 Hz, 6H). 1.39 (m, 4H), 1.30-1.23 (m, 4H), 0.88 (m, 2H)

### Synthesis of Compounds Prepared from Intermediate Ae

### Compound 1015

(2R,6S,12Z,13aS,14aR,16aS)-2-{[7-hydroxy-8-methyl-2-(propan-2-yloxy)quinolin-4-yl]oxy}-N-[(1-methylcyclopropyl)sulfonyl]-6-{[(1-methyl-1H-pyrazol-3-yl)carbonyl]amino}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide

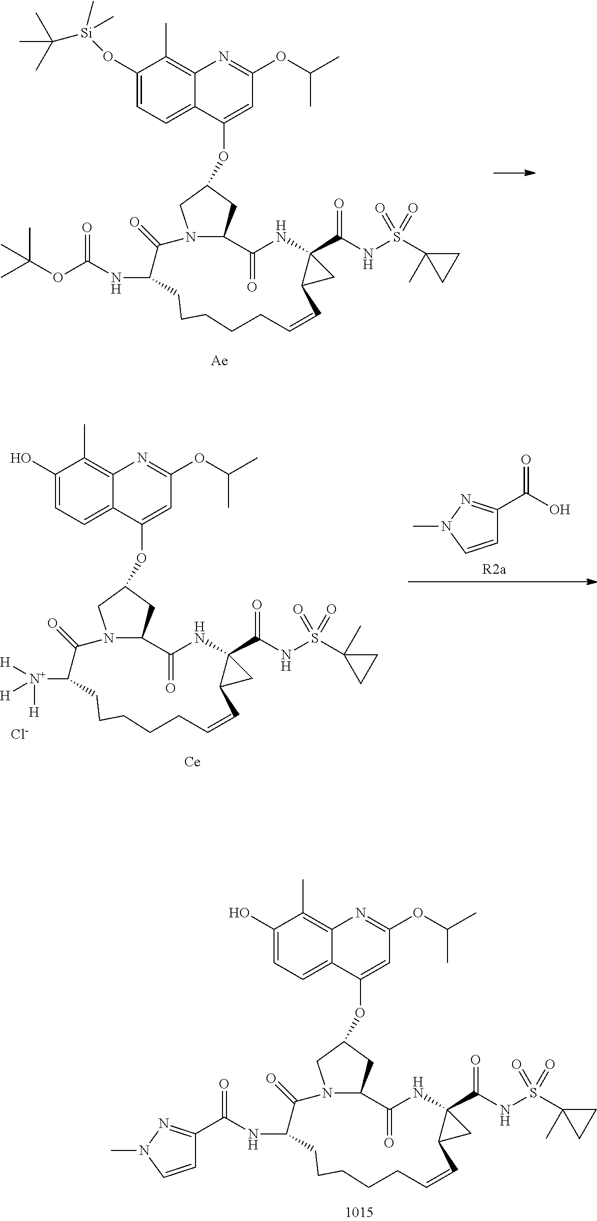

Starting material Ae (302 mg, 0.331 mmol) is dissolved in 1 N HCl/dioxane (3 mL) and this solution is stirred for 1 h at RT. The reaction mixture is concentrated in vacuo to give Ce which is used as such.

Acid R2a (9.3 mg, 0.074 mmol) is dissolved in DCM (2 mL), then TEA (41 μL, 0.295 mmol) is added followed by TBTU (21.7 mg, 0.068 mmol). This solution is stirred for 15 mins, after which the amine hydrochloride Ce (50 mg, 0.059 mmol) is added. The resulting solution is stirred at RT for 16 h and then concentrated. The residual is dissolved in DMSO. The resulting solution is filtered through a Millex filter and purified by prep HPLC (Ammonium formate/MeOH). The pure fractions are combined, concentrated redissolved in MeCN and water, frozen and lyophilized to provide compound 1015.

FIA M.S. (electrospray): 804.5 (M−H)⁻

Retention time (min): 5.2 min

¹H NMR (400 MHz, DMSO-d₆): δ 9.57 (s, 1H), 7.76 (d, 1H, J=2.4 Hz), 7.67 (bs, 1H). 7.64 (d, 1H, J=9 Hz), 6.88 (d, 1H, J=9 Hz), 6.59 (d, 1H, J=2.1 Hz), 6.24 (s, 1H), 5.47 (S, 1H, J=6.1 Hz), 5.41-5.31 (m, 3H) 4.81-4.73 (m, 1H), 4.46 (dd, 1H, J=7.8, 7.6 Hz), 4.30-4.13 (m, 2H) 3.90 (s, 3H), 2.46-2.41 (m, 2H), 2.38 (s, 3H), 2.01-1.69 (m, 4H), 1.59-1.53 (m, 3H), 1.48-1.33 (m, 15H), 1.24-1.12 (m, 4H), 0.64-0.60 (m, 2H)

### Synthesis of Compounds Prepared from Intermediate Af

### Compound 1003

(2R,6S,12Z,13aS,14aR,16aS)-2-{[7-chloro-8-methyl-2-(propan-2-yloxy)quinolin-4-yl]oxy}-N-[(1-methylcyclopropyl)sulfonyl]-6-{[(1-methyl-1H-pyrazol-3-yl)carbonyl]amino}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide

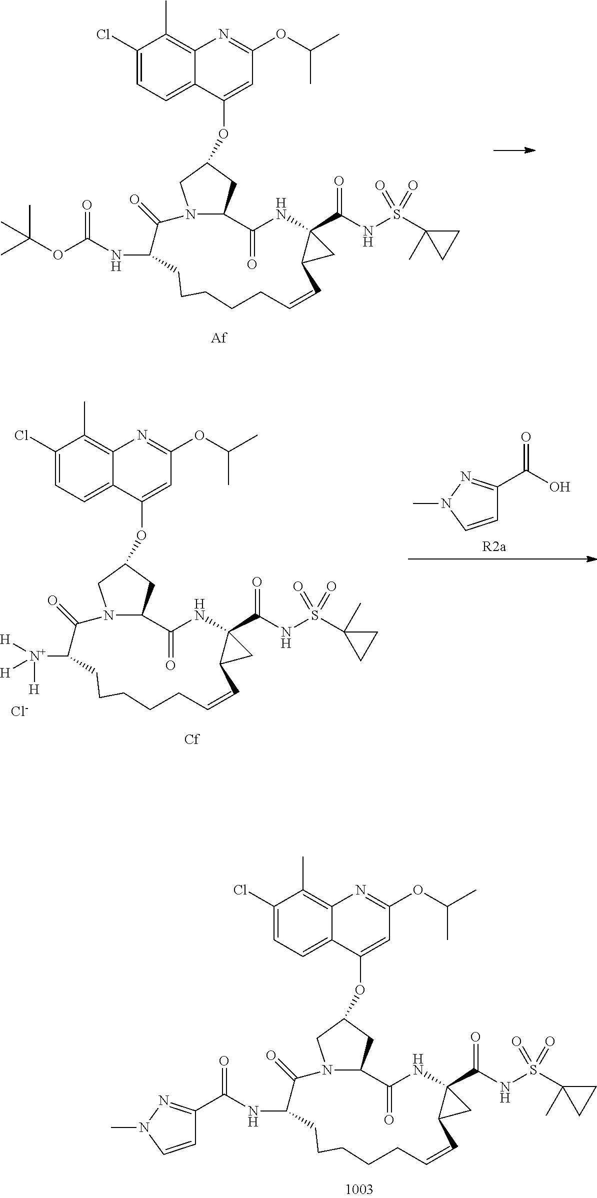

Boc protected macrocyclic amine Af (75 mg, 0.092 mmol) is charged in a vial with a 4 M solution of HCl in dioxane (3 mL). The solution is stirred at RT for 1 h, after which the solution is evaporated to dryness to afford intermediate Cf.

Acid R2a (13.9 mg; 0.110 mmol) is dissolved in DMF (2 mL) and TEA (51.2 μL; 0.367 mmol) and TBTU (34.0 mg; 0.110 mmol) are added and the mixture is stirred for 15 mins. The Boc de-protected macrocyclic amine hydrochloride Cf is dissolved in DMF (1.0 mL) and added to the acid solution and stirred at RT overnight. The resulting solution is filtered through a Millex filter and purified by prep HPLC (ammonium bicarbonate/MeOH). The pure fractions are combined, concentrated, frozen and lyophilized to provide compound 1003.

FIA M.S. (electrospray): 822.5/824.5 (M−H)−, 824.4/826.3 (M+H)⁺

Retention time (min): 7.0 min

¹H NMR (400 MHz, DMSO-d₆): δ 10.83 (s, 1H), 8.92 (s, 1H), 7.86 (d, 1H, J=8.6 Hz), 7.79 (d, 1H, J=5.1 Hz), 7.75 (d, 1H, J=2 Hz), 7.29 (d, 1H, J=8.6 Hz), 6.57 (s, 1H), 6.53 (d, 1H, J=2 Hz), 5.67-5.55 (m, 1H), 5.55-5.44 (m, 2H), 5.12-5.00(m, 1H), 4.62-4.50 (m, 2H), 4.45-4.37 (m, 1H), 4.05-3.95 (m, 1H), 3.88 (s, 3H), 2.66 (s, 3H), 2.40-2.25 (m, 2H), 2.03-1.87 (m, 1H), 1.87-1.72 (m, 1H), 1.60-1.47 (m, 3H), 1.47-1.15 (m, 19H), 0.95-0.78 (m, 2H).

### Synthesis of Compounds Prepared from Intermediate Ah

### Compound 1029

(2R,6S,12Z,13aS,14aR,16aS)-2-{[7-methoxy-8-methyl-2-(2,2,2-trifluoroethoxy)quinolin-4-yl]oxy}-N-[(1-methylcyclopropyl)sulfonyl]-6-{[(1-methyl-1H-pyrazol-3-yl)carbonyl]amino}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide

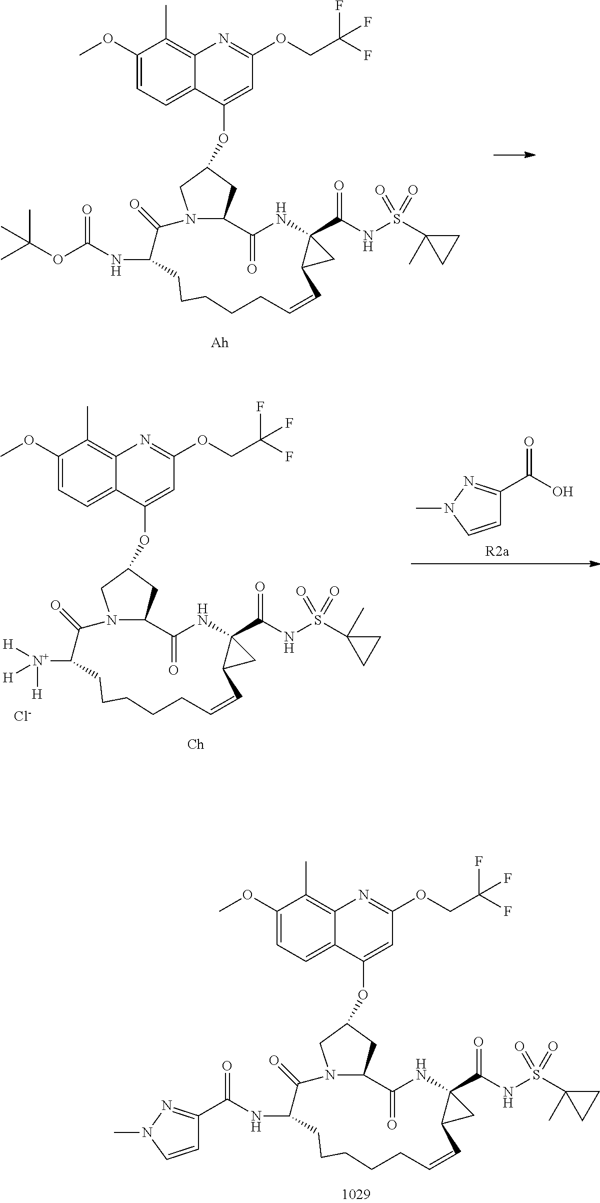

Boc protected macrocyclic amine Ah (50 mg, 0.060 mmol) is charged in a vial with a 4 M solution of HCl in dioxane (2 mL). The solution is stirred at RT for 1 h, after which the solution is evaporated to dryness to give Ch.

Acid R2a (9.03 mg; 0.072 mmol) is dissolved in DMF (2 mL) and TEA (33.3 μL; 0.072 mmol) and TBTU (23.0 mg; 0.072 mmol) are added and the mixture is stirred for 15 mins. The Boc de-protected macrocyclic amine hydrochloride Ch is dissolved in DMF (1.0 mL) and added to the acid solution and stirred at RT overnight. The resulting solution is filtered through a Millex filter and purified by prep HPLC (ammonium bicarbonate/MeOH). The pure fractions are combined, concentrated, frozen and lyophilized to provide compound 1029.

FIA M.S. (electrospray): 858.3 (M−H)⁻, 860.2 (M+H)⁺

Retention time (min): 6.9 min

¹H NMR (400 MHz, DMSO-d₆): δ 10.83 (s, 1H), 8.93 (s, 1H), 7.90 (d, 1H, J=9 Hz), 7.79 (d, 1H, J=7 Hz), 7.75 (d, 1H, J=2.4 Hz), 7.16 (d, 1H, J=9 Hz),), 6.61 (s, 1H), 6.57 (d, 1H, J=2.3 Hz), 5.65-5.58 (m 1H), 5.52 (m, 1H), 5.20-5.04 (m, 3H), 4.61-4.54 (m, 2H), 4.43-4.39 (m, 2H), 4.05-4.00 (m, 2H), 3.89 (d, 6H, J=5.8 Hz), 2.67-2.60 (m, 1H), 2.45 (s, 3H), 2.40-2.29 (m, 2H), 1.97-1.91 (m, 1H), 1.83-1.75 (m, 1H), 1.60-1.49 (m, 3H), 1.43-1.33 (m, 7H), 1.32-1.23 (m, 3H), 0.92-0.84 (m=2H).

### Synthesis of Compounds Prepared from Intermediate Ac

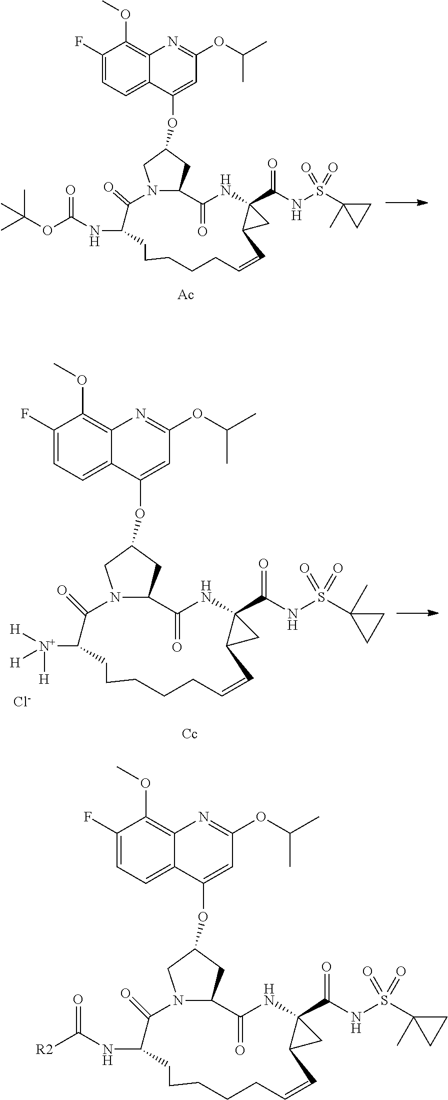

### Compound 1013

(2R,6S,12Z,13aS,14aR,16aS)-2-{[7-fluoro-8-methoxy-2-(propan-2-yloxy)quinolin-4-yl]oxy}-N-[(1-methylcyclopropyl)sulfonyl]-6-{[(1-methyl-1H-pyrazol-3-yl)carbonyl]amino}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide

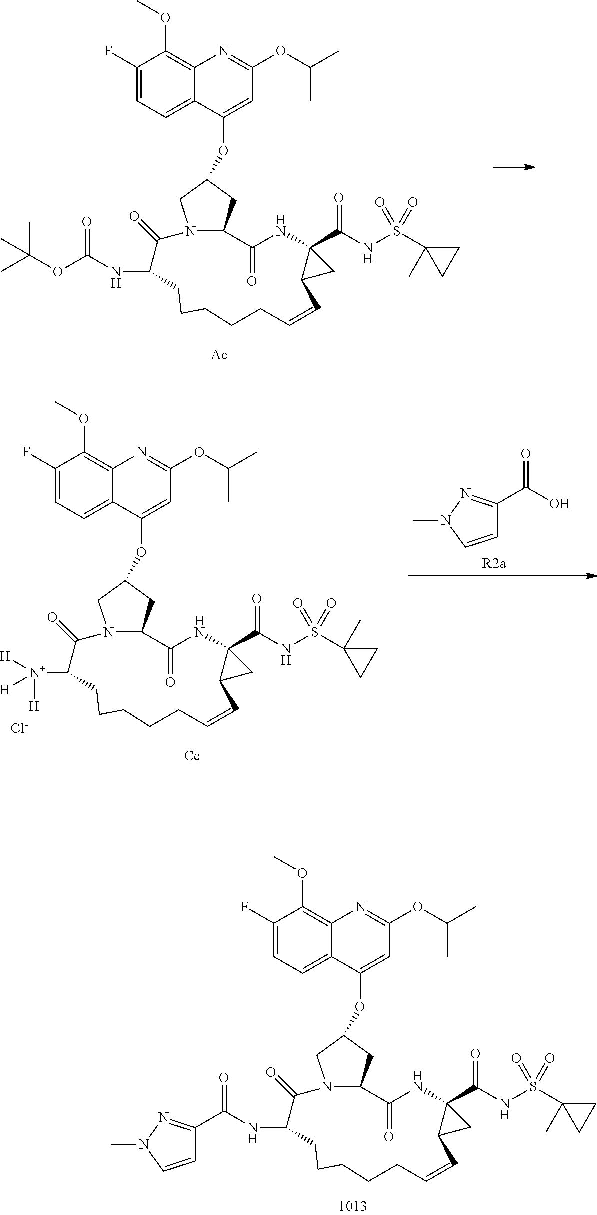

Boc protected macrocyclic amine Ac (84 mg, 0.103 mmol) is charged in a vial with a 4 M solution of HCl in dioxane (3 mL). The solution is stirred at RT for 1 h, after which the solution is evaporated to dryness to afford intermediate Cc.

Acid R2a (15.6 mg; 0.124 mmol, 1.20 equiv) is dissolved in DMF (2 mL) and TEA (57.4 μL; 0.412 mmol, 4.00 equiv) and TBTU (38.1 mg; 0.124 mmol, 1.20 equiv) are added and the mixture is stirred for 15 mins. The Boc de-protected macrocyclic amine hydrochloride Cc is dissolved in DMF (1.0 mL) and added to the acid solution. The reaction is stirred at RT overnight. The resulting solution is filtered through a Millex filter and purified by prep HPLC (ammonium bicarbonate/MeOH) The pure fractions are combined, concentrated, frozen and lyophilized to provide compound 1013.

FIA M.S. (electrospray): 822.5 (M−H)−, 824.4 (M+H)⁺

Retention time (min): 5.5 min

¹H NMR (400 MHz, DMSO-d₆): δ 10.91 (bs, 1H), 8.79 (bs, 1H), 7.81-7.76 (m, 3H), 7.24 (d, 1H, J=8.2 Hz), 6.60 (s, 1H), 6.44 (s, 1H), 5.57 (bs, 1H), 5.49-5.43 (m, 2H), 5.12 (bs, 1H), 4.55 (bs, 2H), 4.40-4.36 (m, 1H), 4.05-3.95 (m, 4H), 3.88 (s, 3H), 2.64-2.56 (m, 1H), 2.35-2.26 (m, 2H), 1.99-1.81 (m, 2H), 1.68-1.19 (m, 21H), 0.82 (bs, 2H).

### Compound 1026

(2R,6S,12Z,13aS,14aR,16aS)-2-{[7-fluoro-8-methoxy-2-(propan-2-yloxy)quinolin-4-yl]oxy}-N-[(1-methylcyclopropyl)sulfonyl]-5,16-dioxo-6-({[1-(2,2,2-trifluoroethyl)-1H-pyrazol-3-yl]carbonyl}amino)-1,2,3,6,7,8,9,10,11,13a,14,15,16,16a-tetradecahydrocyclopropa[e]pyrrolo[1,2-a][1,4]diazacyclopentadecine-14a(5H)-carboxamide

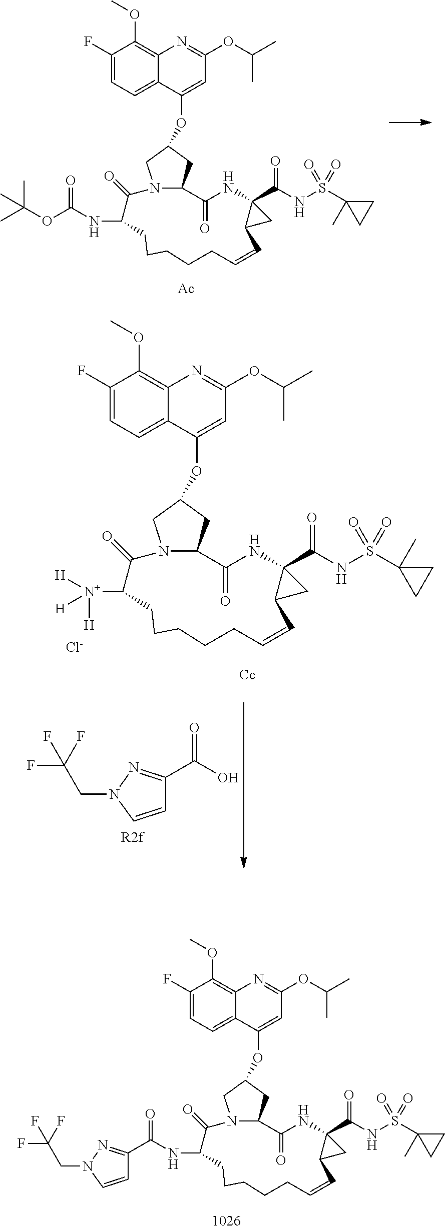

Boc protected macrocyclic amine Ac (722 mg, 0.885 mmol) is charged in a vial with a 4 M solution of HCl in dioxane (5 mL). The solution is stirred at RT for 1 h, after which the solution is evaporated to dryness to provide intermediate Cc.

Acid R2f (17.0 mg; 0.088 mmol, 1.2 equiv) is dissolved in DMF (1 mL) and TEA (40.7 μL; 0.292 mmol, 4.00 equiv) and TBTU (27.0 mg; 0.088 mmol, 1.20 equiv) are added and the mixture is stirred for 15 mins. The Boc de-protected macrocyclic amine hydrochloride Cc (50 mg, 0.075 mmol) is dissolved in DMF (1.0 mL) and added to the acid solution. The reaction is stirred at RT overnight. The resulting solution is filtered through a Millex filter and purified by prep HPLC (ammonium bicarbonate/MeOH) The pure fractions are combined, concentrated, frozen and lyophilized to provide compound 1026.

FIA M.S. (electrospray): 890.4 (M−H)−, 892.4 (M+H)⁺

Retention time (min): 6.0 min

¹H NMR (400 MHz, DMSO-d₆): δ 10.81 (s, 1H), 8.88 (s, 1H), 7.99 (d, 1H, J=6.3 Hz), 7.95 (d, 1H, J=2.4 Hz), 7.80 (d, 1H, J=12.1 Hz), 7.24 (d, 1H, J=8.3 Hz), 6.73 (d 1H, J=2.3 Hz), 6.46 (s, 1H), 5.75-5.55 (m, 1H), 5.50-5.45 (p, 1H, J=6.2 Hz), 5.45 (bs, 1H), 5.24-5.18 (m, 2H), 5.09-5.04 (m, 1H), 4.61 (d, 1H, J=10.9 Hz), 4.51 (m, 1H), 4.38 (dd, 1H, J=7.0 Hz, J=9.4 Hz), 3.95 (s, 3H), 2.67-2.59 (m, 2H), 2.34-2.29 (m, 2H), 2.03-1.91 (m, 1H), 1.77-1.76 (m, 1H), 1.58-1.24 (m, 21H), 0.89-0.84 (m, 2H).

### Compound 1027

(2R,6S,12Z,13aS,14aR,16aS)-6-({[1-(difluoromethyl)-1H-pyrazol-3-yl]carbonyl}amino)-2-{[7-fluoro-8-methoxy-2-(propan-2-yloxy)quinolin-4-yl]oxy}-N-[(1-methylcyclopropyl)sulfonyl]-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide

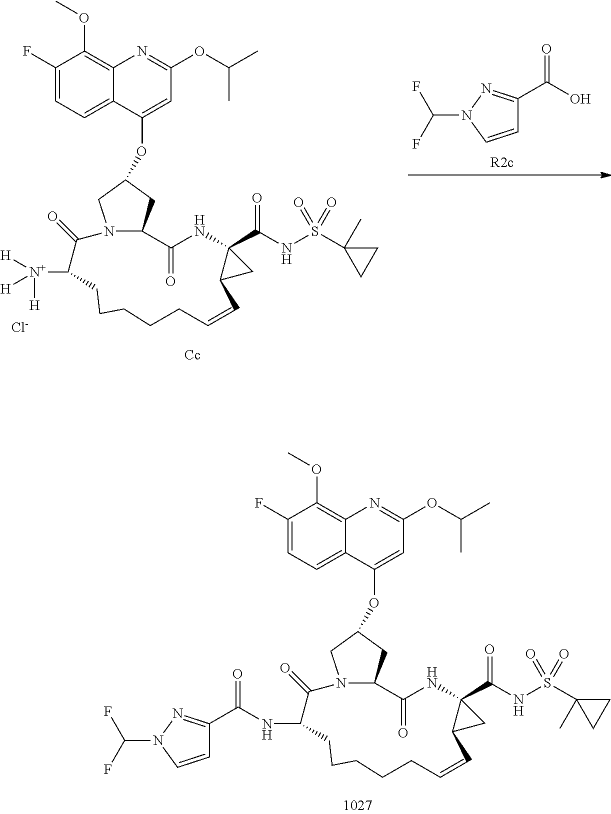

Acid R2c (14.2 mg; 0.088 mmol) is dissolved in DMF (1 mL) and TEA (40.8 μL; 0.292 mmol) and TBTU (27.0 mg; 0.088 mmol) are added and the mixture is stirred for 15 mins. The Boc de-protected macrocyclic amine hydrochloride Cc (55 mg; 0.073 mmol) is dissolved in DMF (1.0 mL) and added to the acid solution. The reaction is stirred at RT overnight. The resulting solution is filtered through a Millex filter and purified by prep HPLC (ammonium bicarbonate/MeOH) The pure fractions are combined, concentrated, frozen and lyophilized to provide compound 1027.

FIA M.S. (electrospray): 858.4 (M−H)−, 860.3 (M+H)⁺

Retention time (min): 5.9 min

¹H NMR (400 MHz, DMSO-d₆): δ 10.83 (s, 1H), 8.88 (s, 1H), 8.40-8.30 (m, 1H), 8.34 (d, 1H, J=2.7 Hz), 7.86 (s, 1H), 7.35-7.7.25 (m, 1H), 7.24 (d, 1H, J=8.2 Hz), 6.84 (d, 1H, J=2.3 Hz), 6.45 (s, 1H), 5.70-5.55 (m, 1H), 5.50-5.40 (m, 2H), 5.15-5.00 (m, 1H), 4.75-4.35 (m, 3H), 3.95 (s, 3H), 2.65-2.50 (m, 1H), 2.40-2.20 (m, 1H), 2.15-1.90 (m, 2H), 1.85-1.57 (m, 1H), 1.55-1.10 (m, 22H), 0.95-0.70 (m, 2H).

### Compound 1028

(2R,6S,12Z,13aS,14aR,16aS)-2-{[7-fluoro-8-methoxy-2-(propan-2-yloxy)quinolin-4-yl]oxy}-N-[(1-methylcyclopropyl)sulfonyl]-6-{[(5-methylthiophen-2-yl)carbonyl]amino}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide

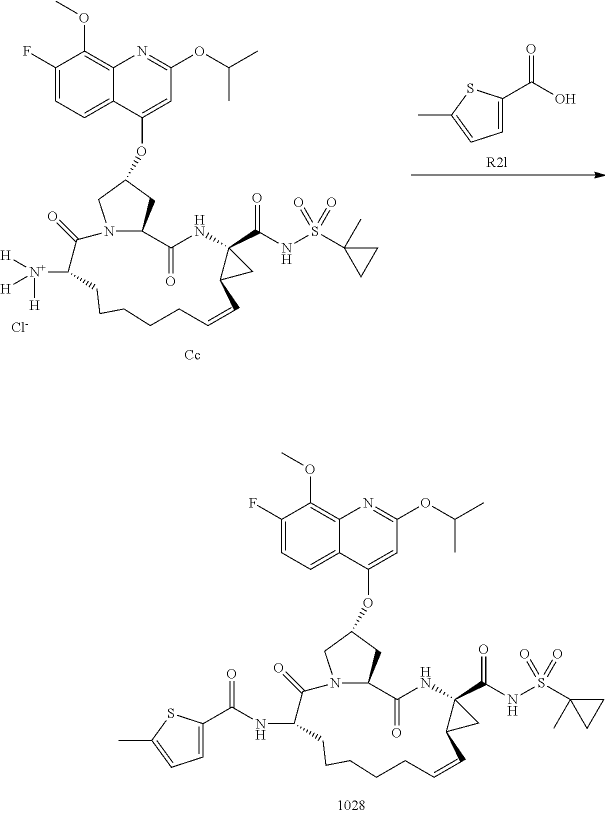

Acid R2l (12.4 mg; 0.088 mmol) is dissolved in DMF (1 mL) and TEA (40.8 μL; 0.292 mmol) and TBTU (27.0 mg; 0.088 mmol) are added and the mixture is stirred for 15 mins. The Boc de-protected macrocyclic amine hydrochloride Cc (55 mg; 0.073 mmol) is dissolved in DMF (1.0 mL) and added to the acid solution. The reaction is stirred at RT overnight. The resulting solution is filtered through a Millex filter and purified by prep HPLC (ammonium bicarbonate/MeOH). The pure fractions are combined, concentrated, frozen and lyophilized to provide compound 1028.

FIA M.S. (electrospray): 838.4 (M−H)−, 840.3 (M+H)⁺

Retention time (min): 6.0 min

¹H NMR (400 MHz, DMSO-d₆): δ 10.82 (s, 1H), 8.88 (s, 1H), 8.55 (s, 1H), 7.73 (d, 1H, J=11.7 Hz), 7.67 (d, 1H, J=3.6 Hz), 7.25 (d, 1H, J=8.2 Hz), 6.80 (d, 1H, J=2.7 Hz), 6.45 (s, 1H), 5.70-5.55 (m, 1H), 5.55-5.40 (m, 2H), 5.15-5.00 (m, 1H), 4.80-4.60 (m, 1H), 4.50-4.30 (m, 2H), 3.96 (s, 3H), 3.95-3.85 (m, 1H), 2.65-2.55 (m, 1H), 2.44 (s, 3H), 2.40-2.25 (m, 1H), 2.05-1.90 (m, 2H), 1.80-1.63 (m, 1H), 1.60-1.19 (m, 21H), 0.90-0.80 (m, 2H).

### Compound 1021

(2R,6S,12Z,13aS,14aR,16aS)-6-({[1-(2-fluoroethyl)-1H-pyrazol-3-yl]carbonyl}amino)-2-{[7-fluoro-8-methoxy-2-(propan-2-yloxy)quinolin-4-yl]oxy}-N-[(1-methylcyclopropyl)sulfonyl]-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide

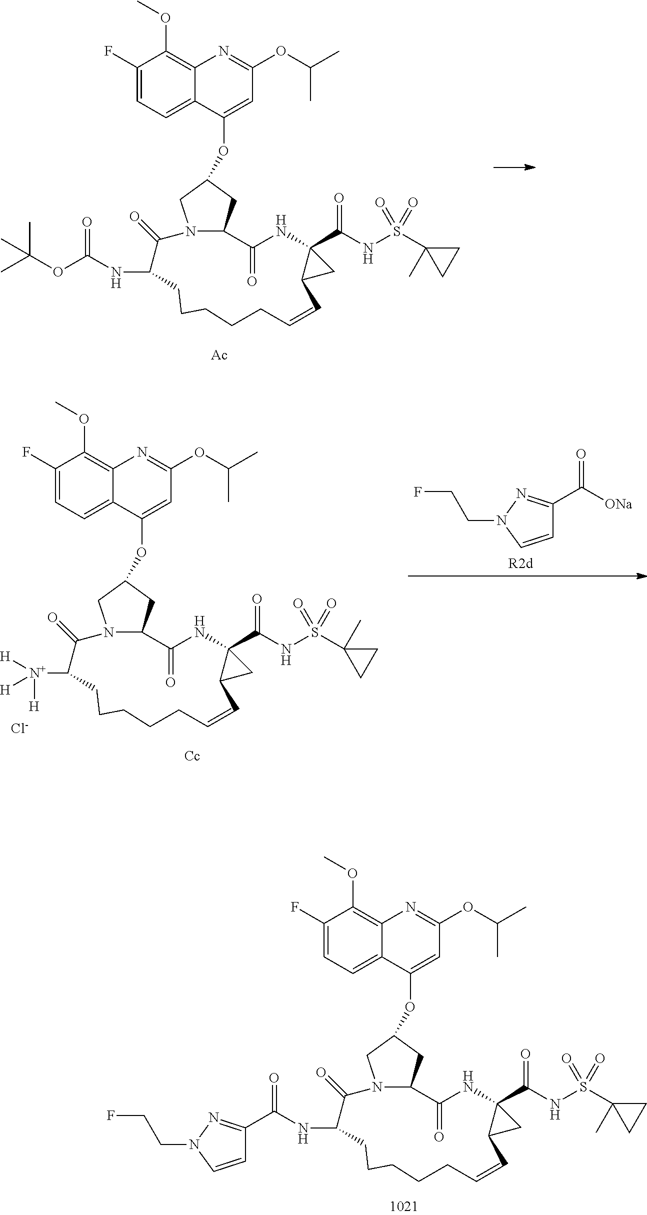

### Compound 1021 is made analogously to the procedure used for the preparation of compound 1028 by using 50 mg of Cc (0.066 mmol) with R2d (15 mg, 0.075 mmol).

FIA M.S. (electrospray): 856.3 (M+H)⁺

Retention time (min): 5.7 min

¹H NMR (400 MHz, DMSO-d₆): δ 10.81 (s, 1H), 8.88 (s, 1H), 7.93-7.88 (m, 1H), 7.84 (d, 1H, J=2.2 Hz), 7.83-7.80 (m, 1H), 7.24 (d, 1H, J=8.1 Hz), 6.64 (d, 1H, J=2.4

Hz), 6.45 (s, 1H), 5.65-5.57 (m, 1H), 5.49-5.41 (m, 2H), 5.11-5.03 (m, 1H), 4.81 (dt, 2H, J=47.3, 4.6 Hz), 4.65-4.61 (m, 1H), 4.55-4.42 (m, 1H), 4.49 (dt, 2H, J=27.7, 4.6 Hz), 4.41 (m, 1H), 4.00-3.91 (m, 1H), 3.95 (s, 3H), 2.65-2.55 (m, 1H), 2.38-2.26 (m, 2H), 1.99-1.91 (m, 1H), 1.82-1.71 (m, 1H), 1.63-1.20 (m, 15H), 1.36 (d, 3H, J=6.0 Hz), 1.34 (d, 3H, J=6.0 Hz), 0.91-0.82 (m, 2H).

### Compound 1022

(2R,6S,12Z,13aS,14aR,16aS)-6-({[1-(2,2-difluoroethyl)-1H-pyrazol-3-yl]carbonyl}amino)-2-{[7-fluoro-8-methoxy-2-(propan-2-yloxy)quinolin-4-yl]oxy}-N-[(1-methylcyclopropyl)sulfonyl]-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide

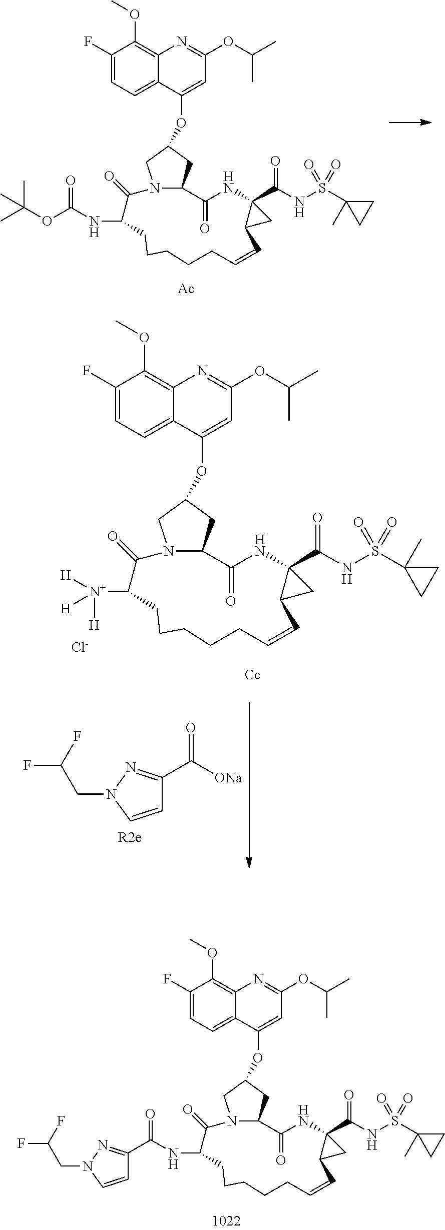

Compound 1022 is made analogously to the procedure used for the preparation of compound 1026 by using 50 mg (0.066 mmol) of Ac and acid R2e (17 mg, 0.080 mmol).

FIA M.S. (electrospray): 874.3 (M+H)⁺

Retention time (min): 5.8 min

¹H NMR (400 MHz, DMSO-d₆): δ 10.82 (s, 1H), 8.88 (s, 1H), 7.95-7.88 (m, 1H), 7.87 (d, 1H, J=2.2 Hz), 7.81-7.78 (m, 1H), 7.24 (d, 1H, J=8.1 Hz), 6.68 (d, 1H, J=2.4 Hz), 6.45 (s, 1H), 6.41 (tt, 1H, J=55.0, 3.6 Hz), 5.65-5.56 (m, 1H), 5.51-5.42 (m, 2H), 5.10-5.04 (m, 1H), 4.69 (td, 2H, J=15.0, 3.6 Hz), 4.64-4.59 (m, 1H), 4.55-4.48 (m, 1H), 4.42-4.35 (m, 1H), 4.00-3.93 (m, 1H), 3.95 (s, 3H), 2.65-2.55 (m, 1H), 2.36-2.27 (m, 2H), 1.99-1.90 (m, 1H), 1.82-1.71 (m, 1H), 1.57-1.19 (m, 15H), 1.36 (d, 3H, J=6.0 Hz), 1.34 (d, 3H, J=6.0 Hz), 0.91-0.82 (m, 2H).

### Compound 1023

(2R,6S,12Z,13aS,14aR,16aS)-2-{[7-fluoro-8-methoxy-2-(propan-2-yloxy)quinolin-4-yl]oxy}-N-[(1-methylcyclopropyl)sulfonyl]-5,16-dioxo-6-({[1-(propan-2-yl)-1H-pyrazol-3-yl]carbonyl}amino)-1,2,3,6,7,8,9,10,11,13a,14,15,16,16a-tetradecahydrocyclopropa[e]pyrrolo[1,2-a][1,4]diazacyclopentadecine-14a(5H)-carboxamide

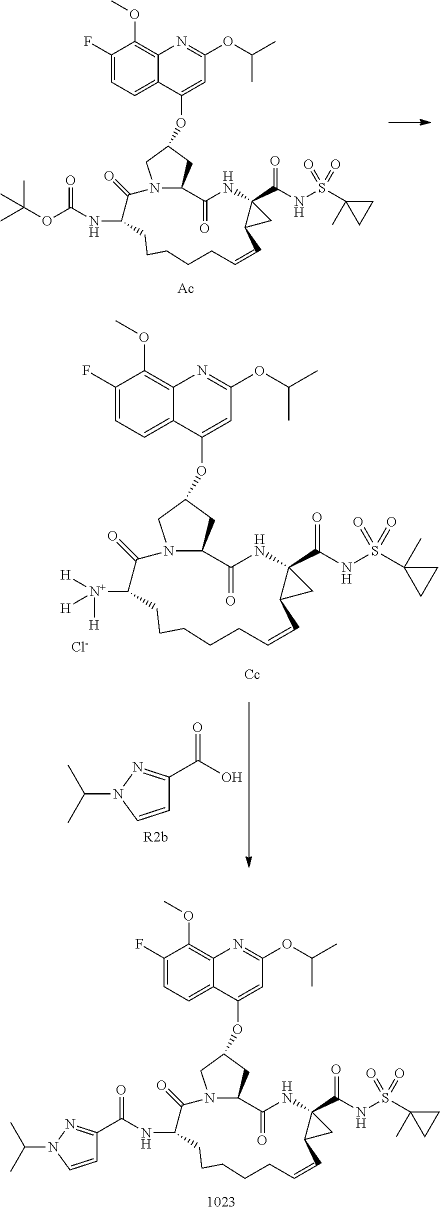

Compound 1023 is made analogously to the procedure used for the preparation of compound 1026 by using 50 mg (0.066 mmol) of Ac and acid R2b (18 mg, 0.080 mmol).

FIA M.S. (electrospray): 852.3 (M+H)⁺

Retention time (min): 6.0 min

¹H NMR (400 MHz, DMSO-d₆): δ 10.82 (s, 1H), 8.88 (s, 1H), 7.84 (d, 1H, J=2.5 Hz), 7.83-7.78 (m, 2H), 7.24 (d, 1H, J=7.9 Hz), 6.60 (d, 1H, J=2.4 Hz), 6.45 (s, 1H), 5.65-5.57 (m, 1H), 5.49-5.42 (m, 2H), 5.10-5.03 (m, 1H), 4.68-4.60 (m, 1H), 4.55 (p, 1H, J=6.7 Hz), 4.52-4.46 (m, 1H), 4.40-4.33 (m, 1H), 3.98-3.92 (m, 1H), 3.95 (s, 3H), 2.64-2.55 (m, 1H), 2.36-2.27 (m, 2H), 2.00-1.91 (m, 1H), 1.82-1.71 (m, 1H), 1.58-1.21 (m, 15H), 1.44 (d, 3H, J=6.7 Hz), 1.43 (d, 3H, J=6.7 Hz), 1.36 (d, 3H, J=6.0 Hz), 1.34 (d, 3H, J=6.0 Hz), 0.90-0.84 (m, 2H).

### Compound 1016

(2R,6S,12Z,13aS,14aR,16aS)-N-(cyclopropylsulfonyl)-2-{[7-fluoro-8-methoxy-2-(propan-2-yloxy)quinolin-4-yl]oxy}-6-{[(1-methyl-1H-pyrazol-3-yl)carbonyl]amino}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide

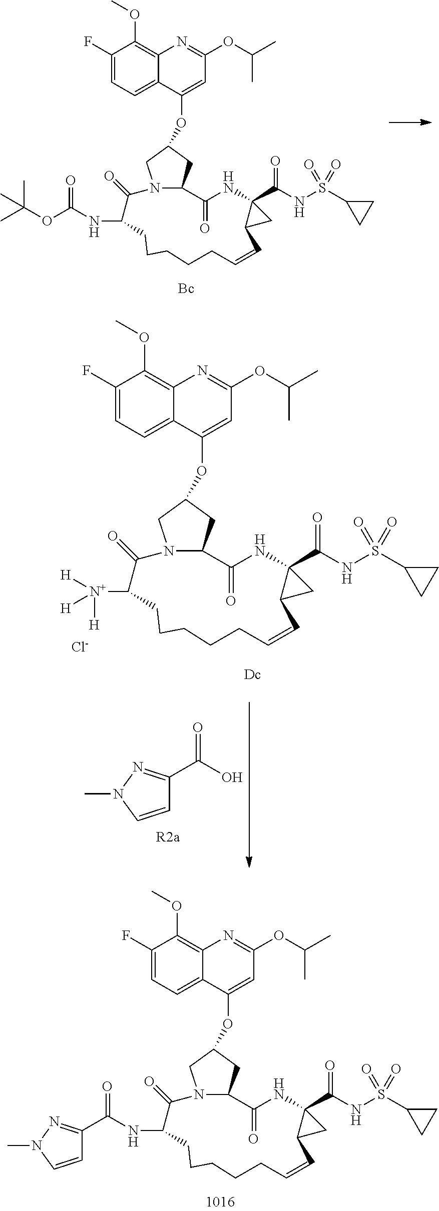

Compound Bc is prepared using Scheme 3, analogously to Compound Ac, but substituting sulfonamide M in place of sulfonamide N in Step 4.

Boc protected macrocyclic amine Bc (85 mg, 0.106 mmol) is charged in a vial and a 4 M solution of HCl in dioxane (3 mL) is added. The solution is stirred at RT for 1 h, after which the solution is evaporated to dryness to afford intermediate Dc.

The 1-methyl-1H-pyrazole-3-carboxylic acid R2a (16 mg, 0.127 mmol, 1.2 equiv) is dissolved in DMF (2 mL), then TEA (59.1 μL, 0.442 mmol, 4 equiv) followed by TBTU (39.2 mg, 0.127 mmol, 1.2 equiv). The reaction mixture is stirred for 15 mins, after which the amine hydrochloride Dc is added in DMF (1 mL). The resulting solution is stirred at RT for 16 h. The solution is then filtered through a Millex filter and purified by prep HPLC. (ammonium bicarbonate/MeOH). The pure fractions are combined, concentrated, frozen and lyophilized to provide compound 1016.

FIA M.S. (electrospray): 808.3 (M−H)−, 810.3 (M+H)⁺

Retention time (min): 5.4 min

¹H NMR (400 MHz, DMSO-d₆): δ 11.04 (s, 1H), 8.76 (s, 1H), 7.83-7.80 (m, 2H), 7.76 (d, 1H, J=2.0 Hz), 7.24 (d, 1H, J=7.8 Hz), 6.59 (d, 1H, J=2.3 Hz), 6.45 (s, 1H), 5.70-5.55 (m, 1H), 5.49-5.43 (p, 1H, J=6.3 Hz), 5.43 (s, 1H), 5.2-5.07 (m, 1H), 4.70-4.50 (m, 1H), 4.49-4.40 (m, 1H), 4.39-4.25 (m, 1H), 4.03-3.85 (m, 1H), 3.95 (s, 3H), 3.89 (s, 3H), 2.95-2.80 (m, 1H), 2.65-2.55 (m, 1H), 2.35-2.19 (m, 2H), 2.0-1.85 (m, 1H), 1.84-1.66 (m, 1H), 1.65-1.5 (m, 3H); 1.49-1.15 (m, 14H); 1.14-0.94 (m, 3H).

### Synthesis of Compounds Using Scheme 2

### Compound 1025

(2R,6S,12Z,13aS,14aR,16aS)-2-{[7,8-dichloro-2-(propan-2-yloxy)quinolin-4-yl]oxy}-N-[(1-methylcyclopropyl)sulfonyl]-6-{[(1-methyl-1H-pyrazol-3-yl)carbonyl]amino}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide

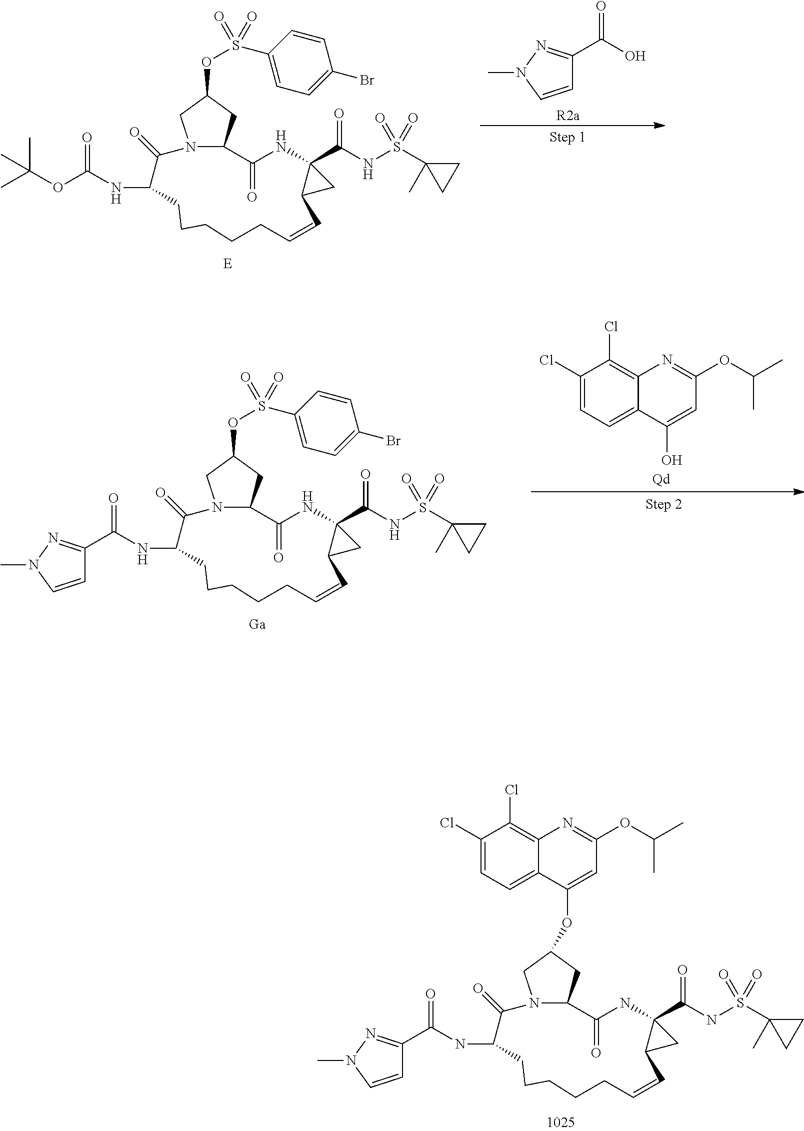

### Step 1: Coupling with R2a

Macrocyclic Brosylate E (1.01 g, 1.26 mmol) is dissolved in 4 N HCl/dioxanes (5 mL) then stirred for 45 mins and concentrated in vacuo. The residue is redissolved in DCM (10 mL), TEA (0.90 mL, 6.5 mmol), TBTU (485 mg, 1.51 mmol) and 1-methyl-1H-pyrazole-3-carboxylic acid (R2a, 206 mg, 1.64 mmol) are added. The reaction mixture is stirred for 4 h at RT. The reaction mixture is concentrated in vacuo and the resulting material purified by flash chromatography using DCM/MeOH (0-10%). The pure fractions are combined and concentrated in vacuo to give intermediate Ga.

### Step 2: Brosylate Displacement with Hydroxy Quinoline Qd

Intermediate Ga (100 mg, 0.123 mmol), and hydroxy quinoline Qd (37 mg, 0.136 mmol) are dissolved in NMP (2 mL), Cs₂CO₃ (120.7 mg, 0.37 mmol) is added and the mixture is heated to 80° C. for 16 h. The material is purified by prep HPLC (ammonium formate/MeOH). The product containing fractions are combined, concentrated in vacuo, redissolved in CH₃CN/H₂O, frozen and lyophilized to give compound 1025.

FIA M.S. (electrospray): 844.2 (M+H)⁺, 842.3 (M−H)⁻

Retention time (min): 7.1 min

¹H NMR (400 MHz, DMSO-d₆): δ 10.83 (bs, 1H), 8.92 (bs, 1H), 7.98 (d, 1H, J=9.0 Hz), 7.80 (bs, 1H), 7.74 (d, 1H, J=2.0 Hz), 7.46 (d, 1H, J=9.0 Hz), 6.68 (bs, 1H), 6.53-6.50 (m, 1H), 5.64-5.57 (m, 2H), 5.54 (S, 1H, J=5.9 Hz), 5.10-5.02 (m, 1H), 4.61-4.39 (m, 3H), 4.02-3.95 (m, 1H), 3.88 (s, 3H), 2.64-2.57 (m, 2H), 2.39-2.26 (m, 1H), 1.99-1.88 (m, 2H), 1.84-1.75 (m, 1H), 1.61-1.47 (m, 3H), 1.45-1.19 (m, 8H), 1.42 (d, 3H, J=5.9 Hz), 1.40 (d, 3H, J=5.9 Hz), 1.40 (s, 3H), 0.92-0.84 (m, 2H).

### Compound 1024

(2R,6S,12Z,13aS,14aR,16aS)-2-{[8-bromo-7-methyl-2-(propan-2-yloxy)quinolin-4-yl]oxy}-N-[(1-methylcyclopropyl)sulfonyl]-6-{[(1-methyl-1H-pyrazol-3-yl)carbonyl]amino}-5,16-dioxo-1,2,3,6,7,8,9,10,11,13a,14,15,16,16a-tetradecahydrocyclopropa[e]pyrrolo[1,2-a][1,4]diazacyclopentadecine-14a(5H)-carboxamide

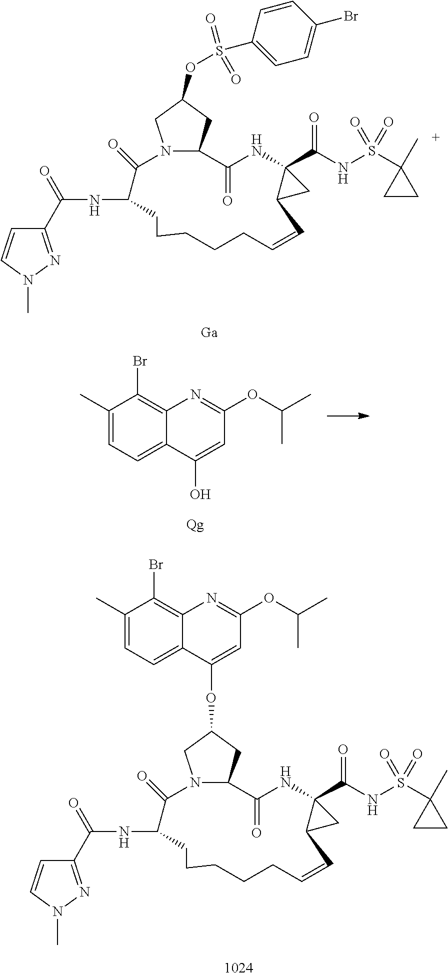

The macrocylic intermediate Ga (75.0 mg, 0.093 mmol) is dissolved in NMP (2 mL) along with hydroxylquinoline Qg (30.2 mg, 0.102 mmol) and cesium carbonate (90.5 mg, 0.28 mmol). The mixture is warmed at 80° C. for 20 h, then filtered with a Millex filter and purified directly by prep HPLC (MeOH, ammonium formate). The appropriate fractions are combined, frozen and lyophilized to give compound 1024.

FIA M.S. (electrospray): 868.2 (M+H)⁺, 866.3 (M−H)⁻.

Retention time (min): 7.0 min

¹H NMR (400 MHz, DMSO-d₆): δ 10.82 (bs, 1H), 8.91 (bs, 1H), 7.85 (d, 1H, J=8.2 Hz), 7.76 (bs, 1H), 7.75 (d, 1H, J=2.4 Hz), 7.25 (d, 1H, J=8.6 Hz), 6.56 (s, 1H), 6.54 (d, 1H, J=2.0 Hz), 5.59 (bs, 1H), 5.54 (S, 1H, J=6.0 Hz), 5.48 (bs, 1H), 5.10 (bs, 1H), 4.62-4.49 (m, 2H), 4.42 (dd, 1H, J=8.8, 7.2 Hz), 4.06-3.98 (m, 1H), 3.88 (s, 3H), 2.63-2.51 (m, 2H), 2.54 (s, 3H), 2.42-2.34 (m, 1H), 1.99-1.88 (m, 2H), 1.61-1.49 (m, 3H), 1.47-1.39 (m, 5H), 1.42 (d, 3H, J=6.0 Hz), 1.41 (d, 3H, J=6.0 Hz), 1.37 (s, 3H), 1.32-1.19 (m, 4H), 0.91-0.81 (m, 2H).

### HCV Replicon RNA Replication Assay (NS3 Protease Variants)

### HCV Replicons:

HCVPV1a and HCVPV1b are subgenomic replicons. HCVPV1a is genotype 1a (strain H77); HCVPV1b is genotype b (Con-1), see Lohman et al., 1999. Science 285: 110-113). Both subgenomic replicons contain a hybrid HCV-poliovirus (PV) 5′UTR, a modified luciferase reporter gene expressed as a luciferase-FMDV2A-neomycin phosphotransferase gene fusion and a NS2-NS5B subgenomic fragment with its 3′UTR. The replication of both HCV NS2-NS5B subgenomic replicons is enhanced by cell-culture adaptive mutations in the NS3 and the NS4B coding regions for the genotype 1a replicon and in the NS3, NS4A and NS5A coding regions for the genotype 1b, as described below. Stable replicon cell lines are established as described, for example, in Lohman et al., 1999. Science 285: 110-113. The amount of luciferase expressed by selected cells directly correlates with the level of HCV replication, as measured by real-time PCR.

SEQ ID NO: 1 is a nucleotide sequence representing the HCV genotype is subgenomic fragment NS2-NS3-NS4A-NS4B-NS5A-NS5B. SEQ ID NO: 1 is 6609 bases wherein nucleotide bases 1-651 of SEQ ID NO: 1 encode NS2, nucleotide bases 652-2544 encode NS3, nucleotide bases 2545-2706 encode NS4A, nucleotide bases 2707-3489 encode NS4B, nucleotide bases 3490-4833 encode NS5A, and nucleotide bases 4834-6606 encode NS5B. NS3 resistance mutation R155K is encoded by the codon of bases 1114-1116 of SEQ ID NO: 1. SEQ ID NO: 2 is the corresponding polypeptide. SEQ ID NO: 2 includes adaptive mutations over reference sequence (GenBank accession number AF009606, residues 811 to 3011 where 811 corresponds to residue 2 in SEQ ID NO: 2) in the NS3 and NS4B coding regions, namely at residues 471, 549, 622, 1000 and 1030 of SEQ ID NO: 2. SEQ ID NO: 2 further includes NS3 resistance mutation R155K which is residue 372.

SEQ ID NO: 3 is a nucleotide sequence representing HCV genotype 1b subgenomic fragment NS2-NS3-NS4A-NS4B-NS5A-NS5B. SEQ ID NO: 3 is 6615 bases wherein nucleotide bases 1-651 of SEQ ID NO: 3 encode NS2, nucleotide bases 652-2544 encode NS3, nucleotide bases 2545-2706 encode NS4A, nucleotide bases 2707-3489 encode NS4B, nucleotide bases 3490-4839 encode NS5A, and nucleotide bases 4840-6612 encode NS5B. NS3 resistance mutation D168V is encoded by the codon of bases 1153-1155 of SEQ ID NO: 3. SEQ ID NO: 4 is the corresponding polypeptide. SEQ ID NO: 4 includes adaptive mutations over reference sequence CON-1 (GenBank accession number AJ238799, residues 811 to 3010) in the NS3, NS4A and NS5A coding regions, namely at residues 326, 751, 882, 1184, 1233, 1346 and 1357 of SEQ ID NO: 4. SEQ ID NO: 4 further includes NS3 resistance mutation D168V which is residue 385 of SEQ ID NO: 4.

SEQ ID NO: 5 is a nucleotide sequence representing the HCV genotype is subgenomic fragment NS2-NS3-NS4A-NS4B-NS5A-NS5B. SEQ ID NO: 5 is 6609 bases, wherein bases 1-651 of SEQ ID NO: 5 encode NS2, nucleotide bases 652-2544 encode NS3, nucleotide bases 2545-2706 encode NS4A, nucleotide bases 2707-3489 encode NS4B, nucleotide bases 3490-4833 encode NS5A, and nucleotide bases 4834-6606 encode NS5B. NS3 resistance mutation D168V is encoded by the codon of bases 1153-1155 of SEQ ID NO: 5. SEQ ID NO: 6 is the corresponding polypeptide. SEQ ID NO: 6 includes adaptive mutations over reference sequence (GenBank accession number AF009606, residues 811 to 3011 where residue 811 corresponds to residue 2 in SEQ ID NO: 6) in the NS3 and NS4B coding regions, namely at residues 471, 549, 622, 1000 and 1030 of SEQ ID NO: 6. SEQ ID NO: 6 further includes NS3 resistance mutation D168V which is residue 385 of SEQ ID NO: 6.

All amino acid substitutions were generated by site-directed mutagenesis using Quick change (Stratagene, La Jolla, Calif.) according to the manufacturer's instructions.

HCV replicon RNA replication assay: To generate cell lines harboring the replicon containing the NS3 substitutions, Huh-7 cells are electroporated with 1-10 μg of purified in vitro transcripts and stable cell lines are selected in the presence of G418 (0.25 mg/ml).

The stable HCV replicon cells are maintained in Dulbecco's Modified Eagle Medium (DMEM) supplemented with 10% FBS and 0.25 mg/ml G418 (standard medium). During the assay, DMEM supplemented with 10% FBS, containing 0.5% DMSO and lacking neomycin are used as assay medium.

For the assay, the cell stocks are trypsinized and diluted in assay medium to distribute 70 μl (8,000 ells) in black 96-well plates. The plates are then incubated at 37° until compound addition. The test compound in 100% DMSO is first diluted in assay medium to a final DMSO concentration of 0.5%. Serial dilutions are prepared in assay medium to generate nine-concentration dose response curves. A fixed volume from each well of the compound dilution plate is transferred to a corresponding well of the cell culture plate. The cell culture plate is incubated at 37° C. with 5% CO₂ for 72 hours. Following the 72 h incubation period, the medium is aspirated from the 96-well assay plate and a volume of 50 μl of 1× Glo Lysis Buffer (Promega) is added to each well. The luciferase activity is determined using Bright-Glo luciferase substrate (Promega) according to the manufacturers instructions and the luminescence is detected on a Packard Topcount instrument. The luminescence (CPS) in each well of the culture plate is a measure of the amount of HCV RNA replication in the presence of various concentrations of inhibitor. The % inhibition is calculated for each inhibitor concentration and used to determine the concentration that results in 50% inhibition of HCV replication (EC₅₀).

Table 1 shows the EC₅₀ (nM) for the compounds of the invention when tested in the HCV replicon RNA replication assay for the R155K 1a, D168V 1b and D168V 1a resistance mutations and as well as the HCV replicon plasmids HCVPV1a and HCVPV1b (referred to as wild type 1a or WT1a, and wild type 1b or WT1b, respectively).

TABLE 1

| Compound | R155K 1a | D168V 1b | D168V 1a | WT 1a | WT 1b |
|---|---|---|---|---|---|
| # | Ec₅₀ (nM) | Ec₅₀ (nM) | Ec₅₀ (nM) | Ec₅₀ (nM) | Ec₅₀ (nM) |
| 1001 | 0.96 | 0.10 | 0.78 | 0.22 | 0.17 |
| 1002 | 1.5 | 0.12 | 2.8 | 0.38 | 0.32 |
| 1003 | 7.7 | 0.12 | 6.9 | 0.24 | 0.21 |
| 1004 | 1.3 | 0.15 | 1.8 | 0.24 | 0.27 |
| 1005 | 0.85 | 0.17 | 1.4 | 0.18 | 0.16 |
| 1006 | 2.2 | 0.17 | 9.6 | 0.19 | 0.18 |
| 1007 | 1.7 | 0.18 | 4.5 | 0.25 | 0.20 |
| 1008 | 1.9 | 0.20 | 6.6 | 0.24 | 0.35 |
| 1009 | 3.6 | 0.24 | 7.2 | 0.43 | 0.46 |
| 1010 | 4.6 | 0.26 | 5.6 | 0.30 | 0.37 |
| 1011 | 3.9 | 0.24 | 6.4 | 0.43 | 0.35 |
| 1012 | 2.1 | 0.31 | 1.9 | 0.29 | 0.24 |
| 1013 | 1.6 | 0.45 | 0.75 | 0.50 | 0.37 |
| 1014 | 8.5 | 0.82 | 6.3 | 1.2 | 1.2 |
| 1015 | 0.95 | 0.96 | 6.9 | 0.96 | 1.3 |
| 1016 | 7.9 | 1.0 | 3.9 | 1.5 | 1.1 |
| 1017 | 1.2 | 1.5 | 2.7 | 1.1 | 1.1 |
| 1018 | 4.2 | 2.4 | 9.8 | 2.7 | 3.3 |
| 1019 | 1.6 | 1.8 | 5.4 | 2.0 | 1.5 |
| 1020 | 2.4 | 0.39 | 3.7 | 0.86 | 0.66 |
| 1021 | 1.9 | 0.53 | 0.93 | 0.34 | 0.48 |
| 1022 | 1.3 | 0.62 | 0.97 | 0.42 | 0.41 |
| 1023 | 3.1 | 0.52 | 0.83 | 0.37 | 0.4 |
| 1024 | 9.3 | 0.09 | 1.9 | 0.10 | 0.13 |
| 1025 | 9.3 | 0.11 | 3.8 | 0.22 | 0.17 |
| 1026 | 1.6 | 0.34 | 0.70 | 0.52 | 0.31 |
| 1027 | 4.7 | 0.39 | 2.2 | 0.47 | 0.33 |
| 1028 | 6.5 | 0.52 | 5.5 | 1.1 | 0.69 |
| 1029 | 1.5 | 0.25 | 9.5 | | |

Table 2 shows the EC₅₀(nM) of three compounds currently in clinical trials, namely MK-7009, ITMN-191 and TMC435, when tested in the HCV replicon RNA replication assay described above for activity in each of R155K 1a, D168V 1b, and D168V 1a resistance mutations (using SEQ ID NOS: 1, 3 and 5, respectively) as well as the wildtype sequences for HCV genotypes 1a and 1b.

TABLE 2

| | R155K 1a | D168V 1b | D168V 1a | WT 1a | WT 1b |
|---|---|---|---|---|---|
| Compound | Ec₅₀ (nM) | Ec₅₀ (nM) | Ec₅₀ (nM) | Ec₅₀ (nM) | Ec₅₀ (nM) |
| 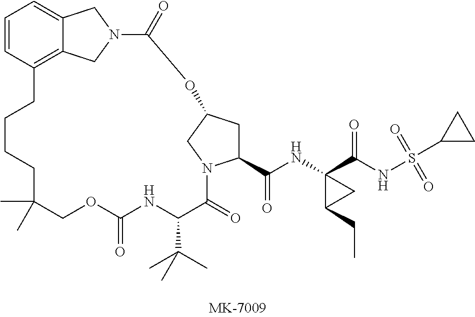 | 1200 | 4400 | 3900 | 0.4 | 1.2 |
| 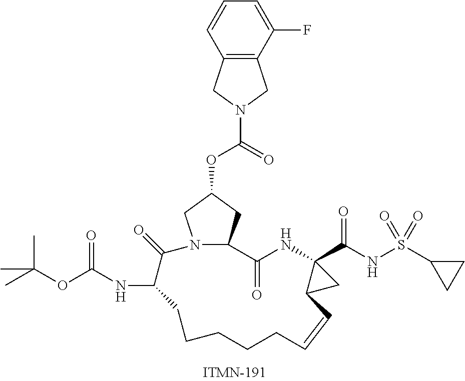 | 690 | 120 | 180 | 2.1 | 5.4 |
| 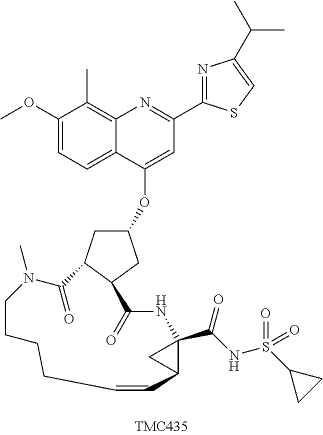 | 300 | 4400 | 3500 | 0.7 | 1.2 |

Each reference, including all patents, patent applications, and publications cited in the present application is incorporated herein by reference in its entirety, as if each of them is individually incorporated. Further, it would be appreciated that, in the above teaching of invention, the skilled in the art could make certain changes or modifications to the invention, and these equivalents would still be within the scope of the invention defined by the appended claims of the application.

What is claimed is:

1. A compound selected from the group consisting of:

| Compound # | Structure |
|---|---|
| 1001 | 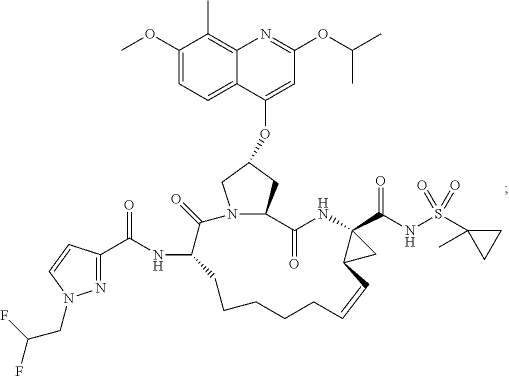 |
| 1002 | 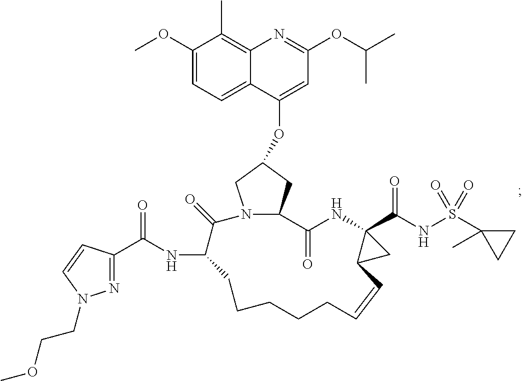 |
| 1003 | 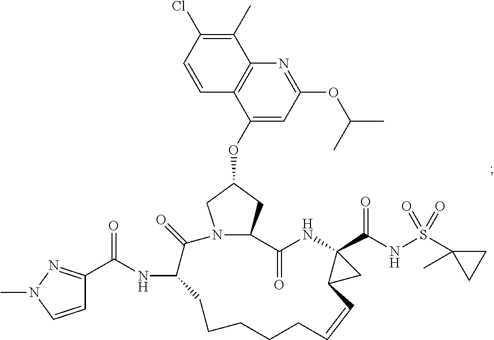 |
| 1004 | 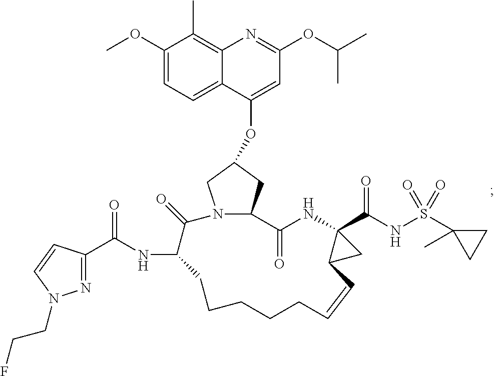 |
| 1005 | 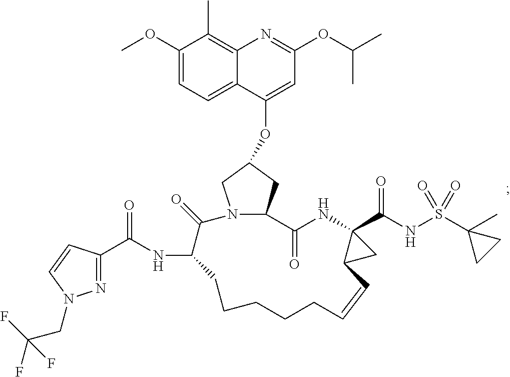 |
| 1006 | 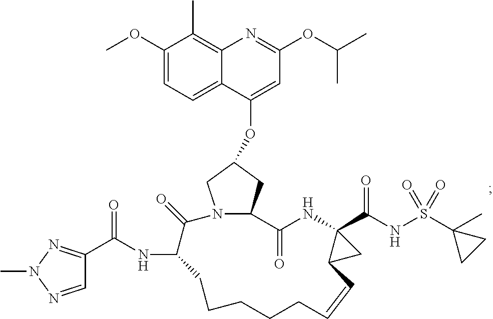 |
| 1007 | 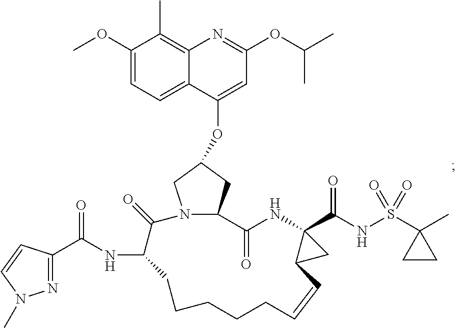 |
| 1008 | 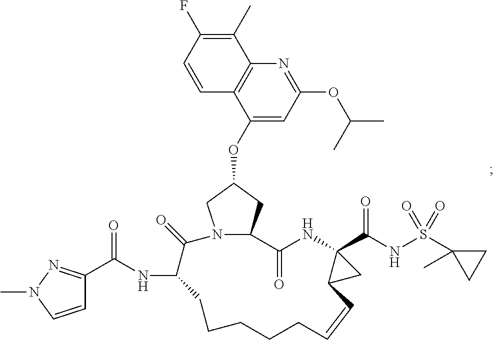 |
| 1009 | 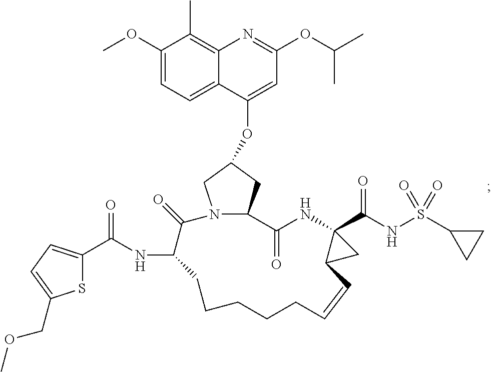 |
| 1010 | 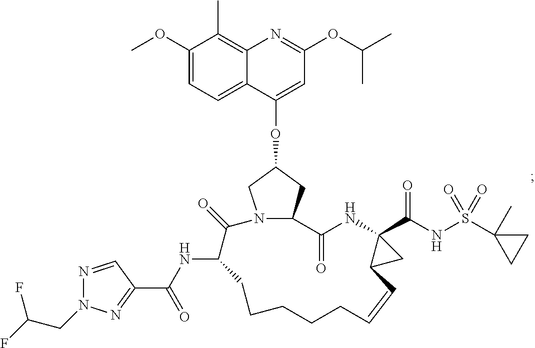 |
| 1011 | 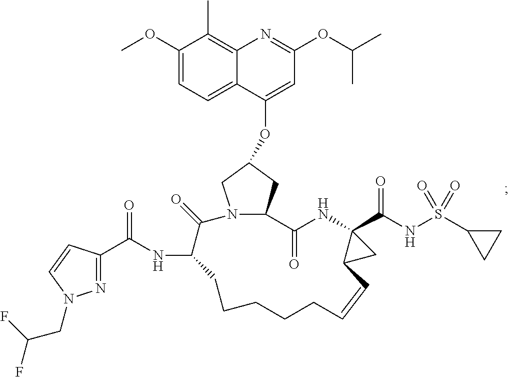 |
| 1012 | 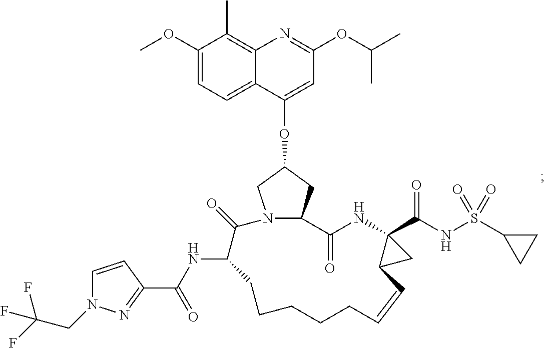 |
| 1013 | 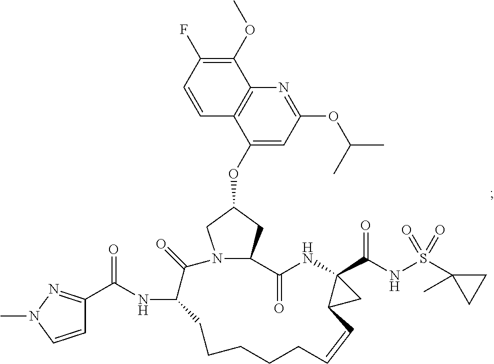 |
| 1014 | 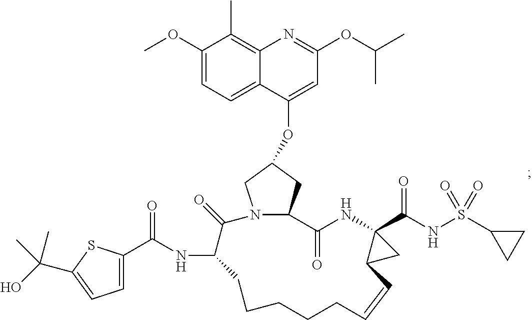 |
| 1015 | 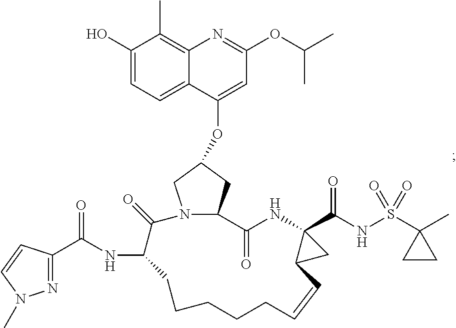 |
| 1016 | 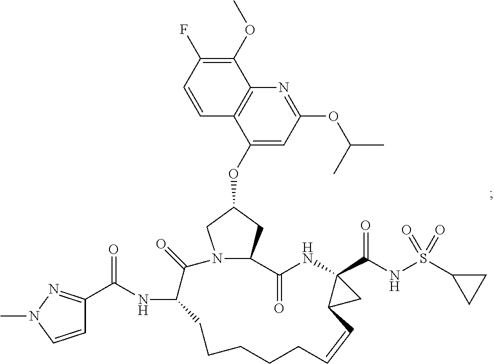 |
| 1017 | 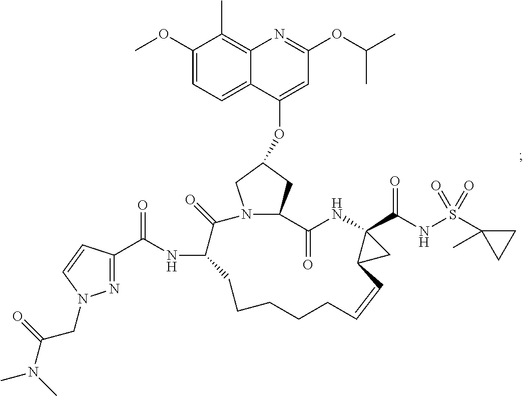 |
| 1018 | 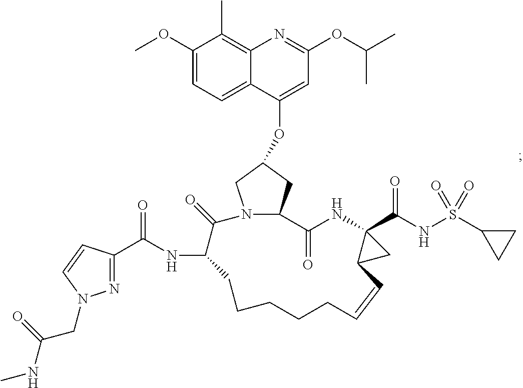 |
| 1019 | 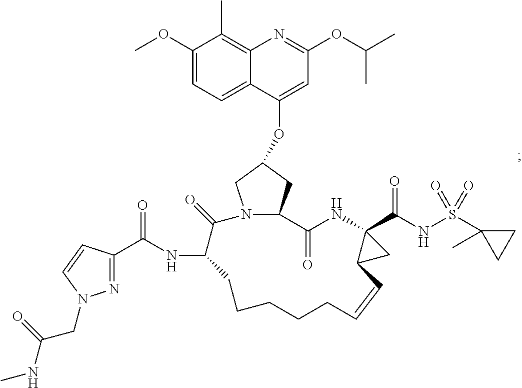 |
| 1020 | 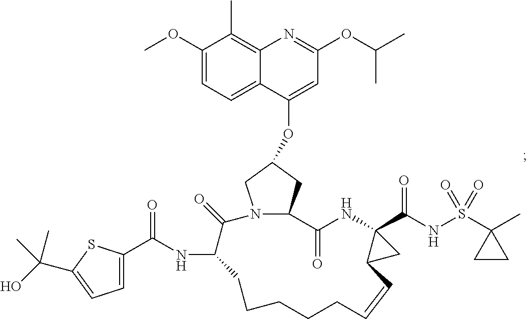 |
| 1021 | 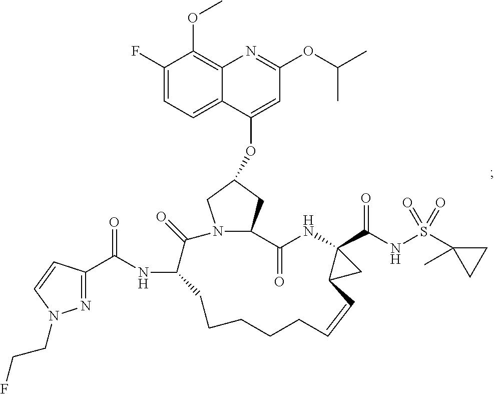 |
| 1022 | 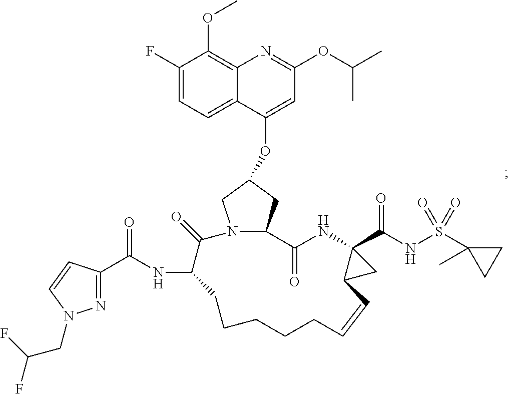 |
| 1023 | 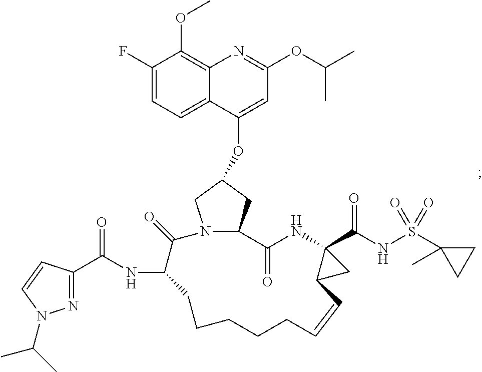 |
| 1024 | 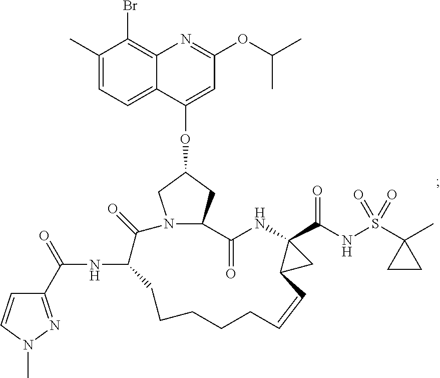 |
| 1025 | 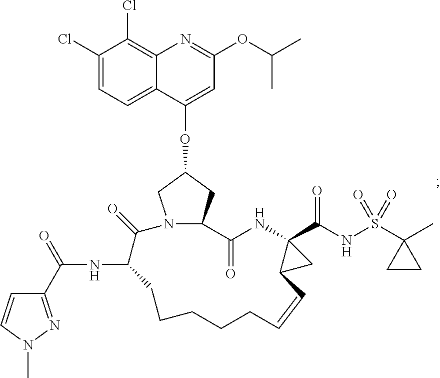 |
| 1026 | 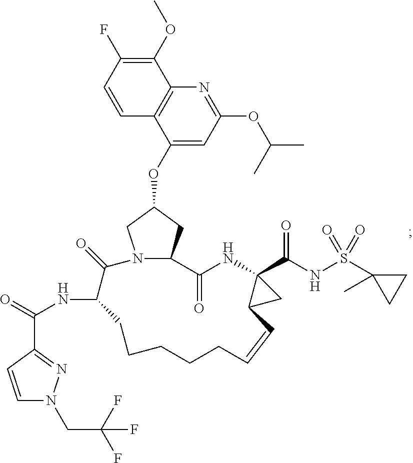 |
| 1027 | 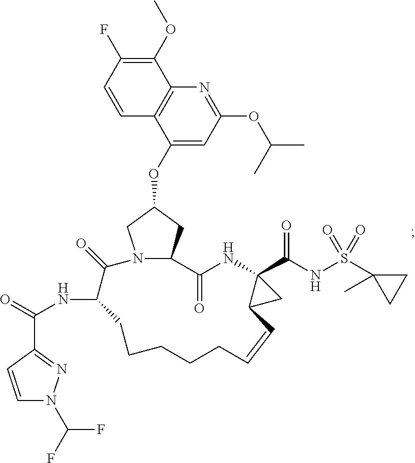 |
| 1028 | 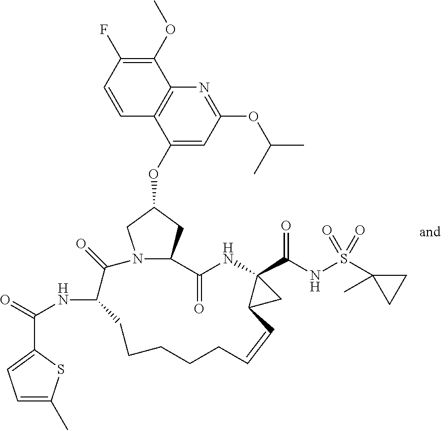 |
| 1029 | 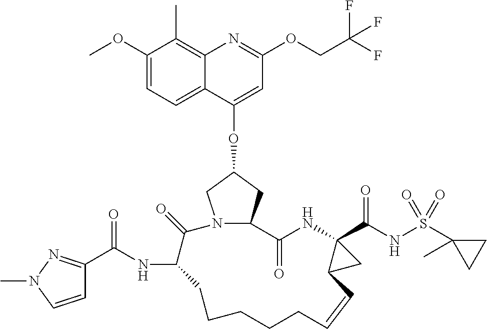 |

or a salt thereof.

2. A compound selected from the group consisting of:

| Compound # | Structure |
|---|---|
| 1001 | 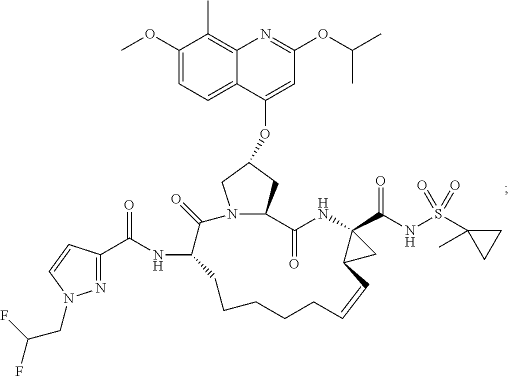 |
| 1002 | 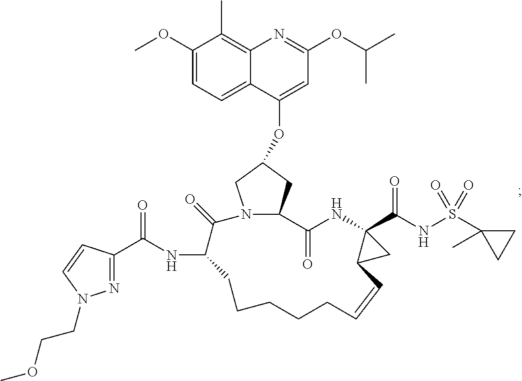 |
| 1004 | 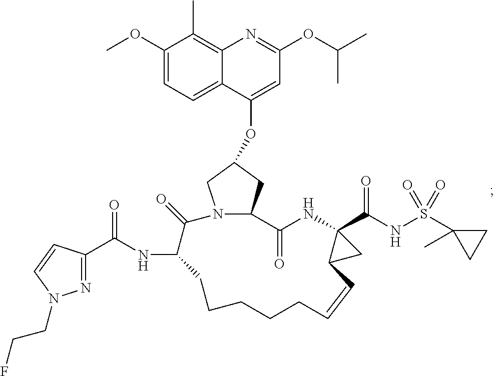 |
| 1005 | 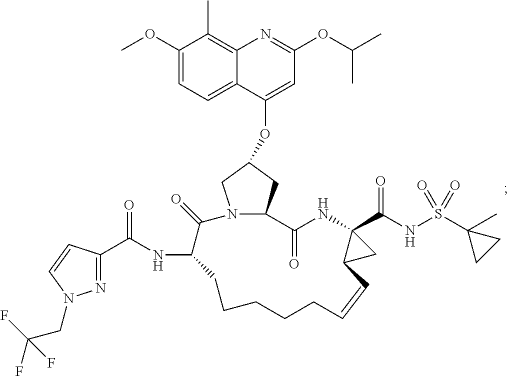 |
| 1006 | 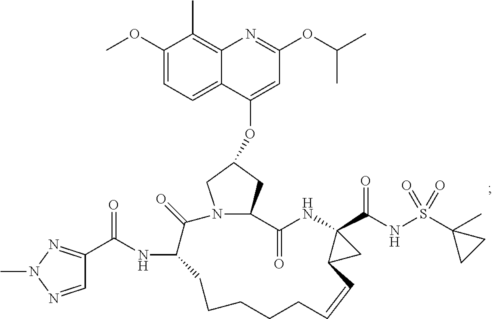 |
| 1007 | 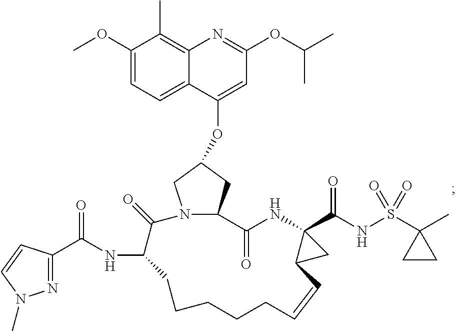 |
| 1010 | 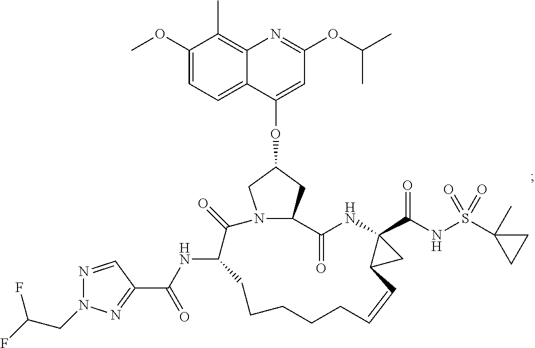 |
| 1011 | 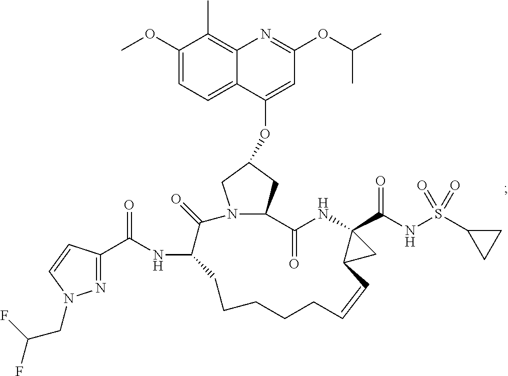 |
| 1012 | 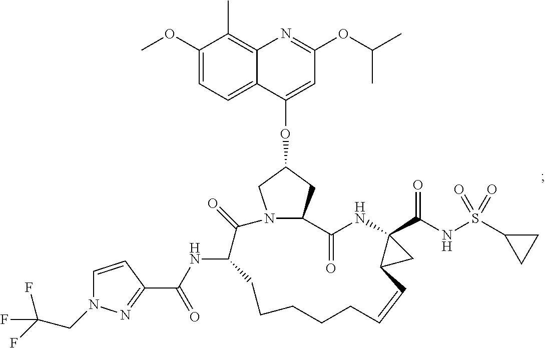 |
| 1017 | 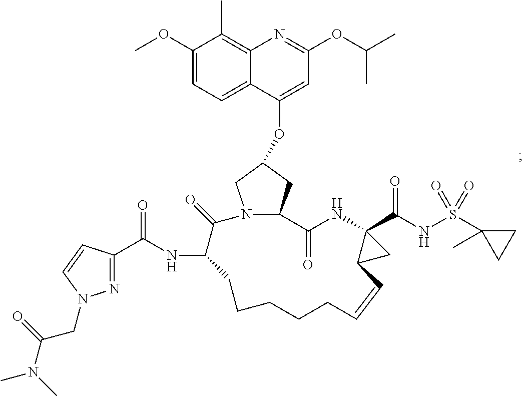 |
| 1018 | 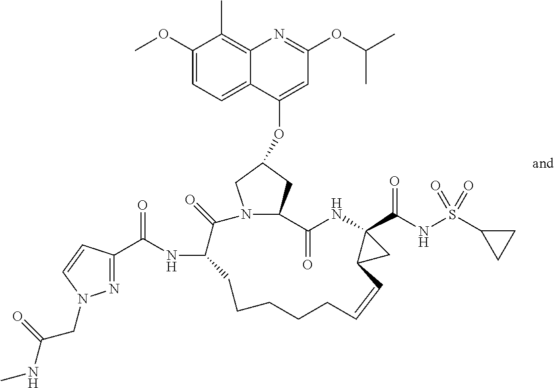 |
| 1019 | 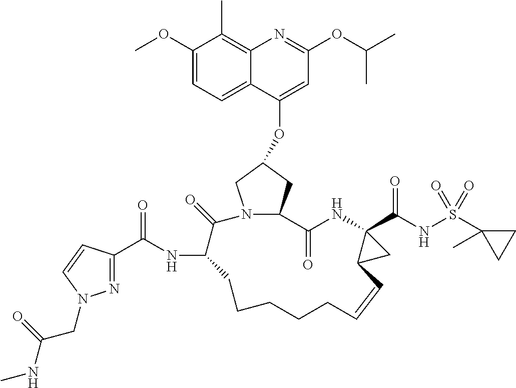 |

or a salt thereof.

3. A compound having the structure: 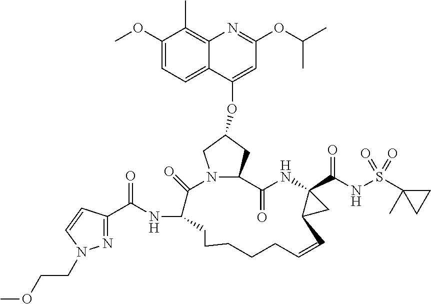
or a pharmaceutically acceptable salt thereof.

4. A compound having the structure: 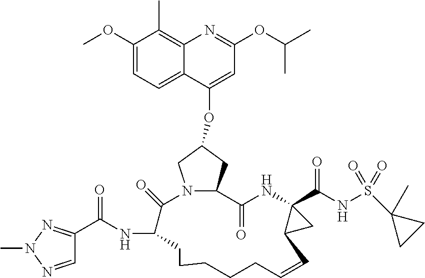
or a pharmaceutically acceptable salt thereof.

5. A compound having the structure: 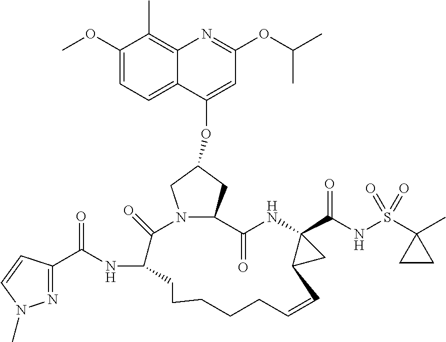
or a pharmaceutically acceptable salt thereof.

6. A compound having the structure: 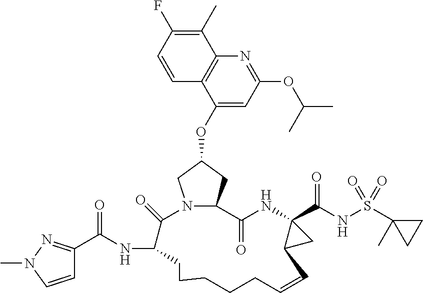
or a pharmaceutically acceptable salt thereof.

7. A compound having the structure: 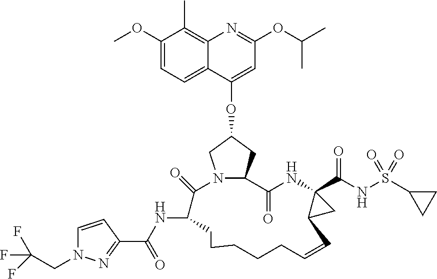
or a pharmaceutically acceptable salt thereof.

8. A compound having the structure: 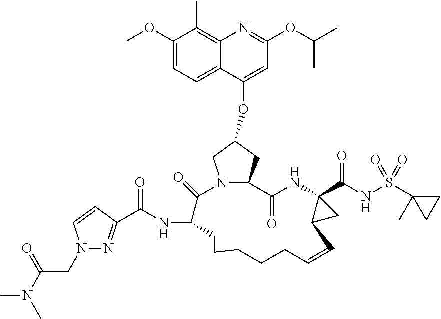
or a pharmaceutically acceptable salt thereof.

9. A compound having the structure: 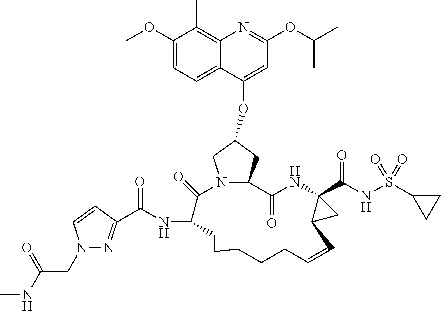
or a pharmaceutically acceptable salt thereof.

10. A compound having the structure: 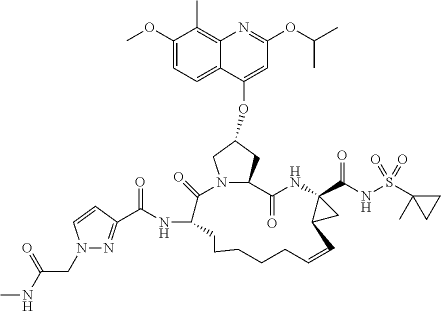
or a pharmaceutically acceptable salt thereof.

11. A compound having the structure: 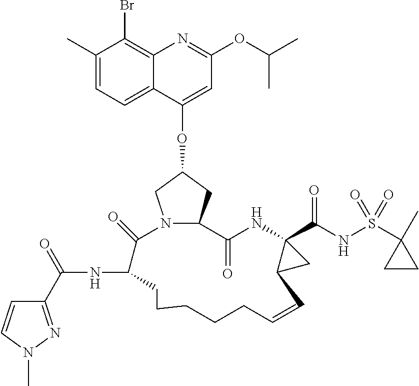
or a pharmaceutically acceptable salt thereof.

12. A pharmaceutical composition comprising an anti-hepatitis C virally effective amount of a compound according to claim 1, or a pharmaceutically acceptable salt thereof, in admixture with at least one pharmaceutically acceptable carrier medium or auxiliary agent.

13. The pharmaceutical composition according to claim 12 further comprising a therapeutically effective amount of at least one other antiviral agent.

14. A method for the treatment or prevention of hepatitis C viral infection in a human being comprising administering to said human being an effective amount of a a compound according to claim 1 or a pharmaceutically acceptable salt thereof.


SEQUENCE LISTING (6 sequences)

SEQ ID NO: 1 - DNA, 6609 bp, HCV
  CDS (1)...(6609)
atggacacggaggtggccgcgtcgtgtggcggcgttgttcttgtcgggttaatggcgctgactctgtcgccatattacaagcgctatatcagctggtgcatgtggtggcttcagtattttctgaccagagtagaagcgcaactgcacgtgtgggttccccccctcaacgtccggggggggcgcgatgccgtcatcttactcatgtgtgtagtacacccgaccctggtatttgacatcaccaaactactcctggccatcttcggacccctttggattcttcaagccagtttgcttaaagtcccctacttcgtgcgcgttcaaggccttctccggatctgcgcgctagcgcggaagatagccggaggtcattacgtgcaaatggccatcatcaagttaggggcgcttactggcacctatgtgtataaccatctcacccctcttcgagactgggcgcacaacggcctgcgagatctggccgtggctgtggaaccagtcgtcttctcccgaatggagaccaagctcatcacgtggggggcagataccgccgcgtgcggtgacatcatcaacggcttgcccgtctctgcccgtaggggccaggagatactgcttgggccagccgacggaatggtctccaaggggtggaggttgctggcgcccatcacggcgtacgcccagcagacgagaggcctcctagggtgtataatcaccagcctgactggccgggacaaaaaccaagtggagggtgaggtccagatcgtgtcaactgctacccaaaccttcctggcaacgtgcatcaatggggtatgctggactgtctaccacggggccggaacgaggaccatcgcatcacccaagggtcctgtcatccagatgtataccaatgtggaccaagaccttgtgggctggcccgctcctcaaggttcccgctcattgacaccctgtacctgcggctcctcggacctttacctggtcacgaggcacgccgatgtcattcccgtgcgccggcgaggtgatagcaggggtagcctgctttcgccccggcccatttcctacttgaaaggctcctcggggggtccgctgttgtgccccgcgggacacgccgtgggcctattcaaggccgcggtgtgcacccgtggagtggctaaagcggtggactttatccctgtggagaacctagggactaccatgagatccccggtgttcacggacaactcctctccaccagcagtgccccagagcttccaggtggcccacctgcatgctcccaccggcagcggtaagagcaccaaggtcccggctgcgtacgcagcccagggctacaaggtgttggtgctcaacccctctgttgctgcaacgctgggctttggtgcttacatgtccaaggcccatggggttgatcctaatatcagggccggggtgagaacaattaccactggcagccccatcacgtactccacctacggcaagttccttgccgacggcgggtgctcagggggtgcttatgacataataatttgtgacgagtgccactccacggatgccacatccatcttgggcatcggcactgtccttgaccaagcagagactgcgggggcgagactggttgtgctcgccactgctacccctccgggctccgtcactgtgccccatcctaacatcgaggaggttgctctgtccaccaccggagagatccccttttacggcaaggctatccccctcgaggtgatcaaggggggaagacatctcatcttctgccactcaaagaagaagtgcgacgagctcgccgcgaagctggtcgcattgggcatcaatgccgtggcctactaccgcggtcttgacgtgtctgtaatcccgaccagcggcaatgttgtcgtcgtgtcgaccgatgctctcatgactggctttaccggcgacttcgactctgtgatagactgcaacacgtgtgtcactcagacagtcgatttcagccttgaccctacctttaccattgagacaaccacgctcccccaggatgctgtctccaggactcaacgccggggcaggactggcagggggaagccaggcatctatagatttgtggcaccgggggagcgcccctccggcatgttcgactcgtccgtcctctgtgagtgctatgacgcgggctgtgcttggtatgagctcacgcccgccgagactacagttaggctacgagcgtacatgaacaccccggggcttcccgtgtgccaggaccatcttgaattttgggagggcgtttttacgggcctcactcatatagatgcccactttttatcccagacaaagcagagtggggagaactttccttacctggtagcgtaccaagccaccgtgtgcgctagggctcaagcccctcccccatcgtgggaccagatgtggaagtgtttgatccgccttaaacccaccctccatgggccaacacccctgctatacagactgggcgctgttcagaatgaagtcaccctgacgcacccaatcaccaaatacatcatgacatgcatgtcggccgacctggaggtcgtcacgagcacctgggtgctcgttggcggcgtcctggctgctctggccgcgtattgcctgtcaacaggctgcgtggtcatagtgggcaggatcgtcttgtccgggaagccggcaattatacctgacagggaggttctctaccaggagttcgatgagatggaagagtgctctcagcacttaccgtacatcgagcaagggatgatgctcgctgagcagttcaagcagaaggccctcggcctcctgcagaccgcgtcccgccatgcagaggttatcacccctgctgtccagaccaactggcagaaactcgaggtcttttgggcgaagcacatgtggaatttcatcagtgggatacaatacttggcgggcctgtcaacgctgcctggtaaccccgccattgcttcattgatggcttttacagctgccgtcaccagcccactgaccactggccaaaccctcctcctcaacatattgggggggtgggtggctgcccagctcgccgcccccggtgccgctactgcctttgtgggtgctggcctagctggcgccgccgtcggcagcgttggactggggaaggtcctcgtggacattcttgcagggtatggcgcgggcgtggcgggagctcttgtagcattcaagatcatgagcggtgaggtcccctccacggaggacctggtcaatctgctgcccgccatcctctcgcctggagcccttgtagtcggtgtggtctgcgcagcaatactgcgccggcacgttggcccgggcgagggggcagtgcaatggatgaaccggctaatagccttcgcctcccgggggaaccatgtttcccccacgcactacgtgccggagagcgatgcagccgcccgcgtcactgccatactcagcagcctcactgtaacccagctcctgaggcgactgcatcagtggataagctcggagtgtaccactccatgctccggttcctggctaagggacatctgggactggatatgcgaggtgctgagcgactttaagacctggctgaaagccaagctcatgccacaactgcctgggattccctttgtgtcctgccagcgcgggtataggggggtctggcgaggagacggcattatgcacactcgctgccactgtggagctgagatcactggacatgtcaaaaacgggacgatgaggatcgtcggtcctaggacgtgcaggaacatgtggagtgggacgttccccattaacgcctacaccacgggcccctgtactccccttcctgcgccgaactataagttcgcgctgtggagggtgtctgcagaggaatacgtggagataaggcgggtgggggacttccactacgtatcgggtatgactactgacaatcttaaatgcccgtgccagatcccatcgcccgaatttttcacagaattggacggggtgcgcctacacaggtttgcgcccccttgcaagcccttgctgcgggaggaggtatcattcagagtaggactccacgagtacccggtggggtcgcaattaccttgcgagcccgaaccggacgtagccgtgttgacgtccatgctcactgatccctcccatataacagcagaggcggccgggagaaggttggcgagagggtcacccccttctatggccagctcctcggctagccagctgtccgctccatctctcaaggcaacttgcaccgccaaccatgactcccctgacgccgagctcatagaggctaacctcctgtggaggcaggagatgggcggcaacatcaccagggttgagtcagagaacaaagtggtgattctggactccttcgatccgcttgtggcagaggaggatgagcgggaggtctccgtacctgcagaaattctgcggaagtctcggagattcgcccgggccctgcccgtctgggcgcggccggactacaaccccccgctagtagagacgtggaaaaagcctgactacgaaccacctgtggtccatggctgcccgctaccacctccacggtcccctcctgtgcctccgcctcggaaaaagcgtacggtggtcctcaccgaatcaaccctatctactgccttggccgagcttgccaccaaaagttttggcagctcctcaacttccggcattacgggcgacaatacgacaacatcctctgagcccgccccttctggctgcccccccgactccgacgttgagtcctattcttccatgccccccctggagggggagcctggggatccggatctcagcgacgggtcatggtcgacggtcagtagtggggccgacacggaagatgtcgtgtgctgctcaatgtcttattcctggacaggcgcactcgtcaccccgtgcgctgcggaagaacaaaaactgcccatcaacgcactgagcaactcgttgctacgccatcacaatctggtgtattccaccacttcacgcagtgcttgccaaaggcagaagaaagtcacatttgacagactgcaagttctggacagccattaccaggacgtgctcaaggaggtcaaagcagcggcgtcaaaagtgaaggctaacttgctatccgtagaggaagcttgcagcctgacgcccccacattcagccaaatccaagtttggctatggggcaaaagacgtccgttgccatgccagaaaggccgtagcccacatcaactccgtgtggaaagaccttctggaagacagtgtaacaccaatagacactaccatcatggccaagaacgaggttttctgcgttcagcctgagaaggggggtcgtaagccagctcgtctcatcgtgttccccgacctgggcgtgcgcgtgtgcgagaagatggccctgtacgacgtggttagcaagctccccctggccgtgatgggaagctcctacggattccaatactcaccaggacagcgggttgaattcctcgtgcaagcgtggaagtccaagaagaccccgatggggttctcgtatgatacccgctgttttgactccacagtcactgagagcgacatccgtacggaggaggcaatttaccaatgttgtgacctggacccccaagcccgcgtggccatcaagtccctcactgagaggctttatgttgggggccctcttaccaattcaaggggggaaaactgcggctaccgcaggtgccgcgcgagcggcgtactgacaactagctgtggtaacaccctcacttgctacatcaaggcccgggcagcctgtcgagccgcagggctccaggactgcaccatgctcgtgtgtggcgacgacttagtcgttatctgtgaaagtgcgggggtccaggaggacgcggcgagcctgagagccttcacggaggctatgaccaggtactccgccccccccggggaccccccacaaccagaatacgacttggagcttataacatcatgctcctccaacgtgtcagtcgcccacgacggcgctggaaagagggtctactaccttacccgtgaccctacaacccccctcgcgagagccgcgtgggagacagcaagacacactccagtcaattcctggctaggcaacataatcatgtttgcccccacactgtgggcgaggatgatactgatgacccatttctttagcgtcctcatagccagggatcagcttgaacaggctcttaactgtgagatctacggagcctgctactccatagaaccactggatctacctccaatcattcaaagactccatggcctcagcgcattttcactccacagttactctccaggtgaaatcaatagggtggccgcatgcctcagaaaacttggggtcccgcccttgcgagcttggagacaccgggcccggagcgtccgcgctaggcttctgtccagaggaggcagggctgccatatgtggcaagtacctcttcaactgggcagtaagaacaaagctcaaactcactccaatagcggccgctggccggctggacttgtccggttggttcacggctggctacagcgggggagacatttatcacagcgtgtctcatgcccggccccgctggttctggttttgcctactcctgctcgctgcaggggtaggcatctacctcctccccaaccgatga

SEQ ID NO: 2 - protein, 2202 aa, HCV
MDTEVAASCGGVVLVGLMALTLSPYYKRYISWCMWWLQYFLTRVEAQLHVWVPPLNVRGGRDAVILLMCVVHPTLVFDITKLLLAIFGPLWILQASLLKVPYFVRVQGLLRICALARKIAGGHYVQMAIIKLGALTGTYVYNHLTPLRDWAHNGLRDLAVAVEPVVFSRMETKLITWGADTAACGDIINGLPVSARRGQEILLGPADGMVSKGWRLLAPITAYAQQTRGLLGCIITSLTGRDKNQVEGEVQIVSTATQTFLATCINGVCWTVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFKAAVCTRGVAKAVDFIPVENLGTTMRSPVFTDNSSPPAVPQSFQVAHLHAPTGSGKSTKVPAAYAAQGYKVLVLNPSVAATLGFGAYMSKAHGVDPNIRAGVRTITTGSPITYSTYGKFLADGGCSGGAYDIIICDECHSTDATSILGIGTVLDQAETAGARLVVLATATPPGSVTVPHPNIEEVALSTTGEIPFYGKAIPLEVIKGGRHLIFCHSKKKCDELAAKLVALGINAVAYYRGLDVSVIPTSGNVVVVSTDALMTGFTGDFDSVIDCNTCVTQTVDFSLDPTFTIETTTLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENFPYLVAYQATVCARAQAPPPSWDQMWKCLIRLKPTLHGPTPLLYRLGAVQNEVTLTHPITKYIMTCMSADLEVVTSTWVLVGGVLAALAAYCLSTGCVVIVGRIVLSGKPAIIPDREVLYQEFDEMEECSQHLPYIEQGMMLAEQFKQKALGLLQTASRHAEVITPAVQTNWQKLEVFWAKHMWNFISGIQYLAGLSTLPGNPAIASLMAFTAAVTSPLTTGQTLLLNILGGWVAAQLAAPGAATAFVGAGLAGAAVGSVGLGKVLVDILAGYGAGVAGALVAFKIMSGEVPSTEDLVNLLPAILSPGALVVGVVCAAILRRHVGPGEGAVQWMNRLIAFASRGNHVSPTHYVPESDAAARVTAILSSLTVTQLLRRLHQWISSECTTPCSGSWLRDIWDWICEVLSDFKTWLKAKLMPQLPGIPFVSCQRGYRGVWRGDGIMHTRCHCGAEITGHVKNGTMRIVGPRTCRNMWSGTFPINAYTTGPCTPLPAPNYKFALWRVSAEEYVEIRRVGDFHYVSGMTTDNLKCPCQIPSPEFFTELDGVRLHRFAPPCKPLLREEVSFRVGLHEYPVGSQLPCEPEPDVAVLTSMLTDPSHITAEAAGRRLARGSPPSMASSSASQLSAPSLKATCTANHDSPDAELIEANLLWRQEMGGNITRVESENKVVILDSFDPLVAEEDEREVSVPAEILRKSRRFARALPVWARPDYNPPLVETWKKPDYEPPVVHGCPLPPPRSPPVPPPRKKRTVVLTESTLSTALAELATKSFGSSSTSGITGDNTTTSSEPAPSGCPPDSDVESYSSMPPLEGEPGDPDLSDGSWSTVSSGADTEDVVCCSMSYSWTGALVTPCAAEEQKLPINALSNSLLRHHNLVYSTTSRSACQRQKKVTFDRLQVLDSHYQDVLKEVKAAASKVKANLLSVEEACSLTPPHSAKSKFGYGAKDVRCHARKAVAHINSVWKDLLEDSVTPIDTTIMAKNEVFCVQPEKGGRKPARLIVFPDLGVRVCEKMALYDVVSKLPLAVMGSSYGFQYSPGQRVEFLVQAWKSKKTPMGFSYDTRCFDSTVTESDIRTEEAIYQCCDLDPQARVAIKSLTERLYVGGPLTNSRGENCGYRRCRASGVLTTSCGNTLTCYIKARAACRAAGLQDCTMLVCGDDLVVICESAGVQEDAASLRAFTEAMTRYSAPPGDPPQPEYDLELITSCSSNVSVAHDGAGKRVYYLTRDPTTPLARAAWETARHTPVNSWLGNIIMFAPTLWARMILMTHFFSVLIARDQLEQALNCEIYGACYSIEPLDLPPIIQRLHGLSAFSLHSYSPGEINRVAACLRKLGVPPLRAWRHRARSVRARLLSRGGRAAICGKYLFNWAVRTKLKLTPIAAAGRLDLSGWFTAGYSGGDIYHSVSHARPRWFWFCLLLLAAGVGIYLLPNR

SEQ ID NO: 3 - DNA, 6615 bp, HCV
  CDS (1)...(6615)
atggaccgggagatggcagcatcgtgcggaggcgcggttttcgtaggtctgatactcttgaccttgtcaccgcactataagctgttcctcgctaggctcatatggtggttacaatattttatcaccagggccgaggcacacttgcaagtgtggatcccccccctcaacgttcgggggggccgcgatgccgtcatcctcctcacgtgcgcgatccacccagagctaatctttaccatcaccaaaatcttgctcgccatactcggtccactcatggtgctccaggctggtataaccaaagtgccgtacttcgtgcgcgcacacgggctcattcgtgcatgcatgctggtgcggaaggttgctgggggtcattatgtccaaatggctctcatgaagttggccgcactgacaggtacgtacgtttatgaccatctcaccccactgcgggactgggcccacgcgggcctacgagaccttgcggtggcagttgagcccgtcgtcttctctgatatggagaccaaggttatcacctggggggcagacaccgcggcgtgtggggacatcatcttgggcctgcccgtctccgcccgcagggggagggagatacatctgggaccggcagacagccttgaagggcaggggtggcgactcctcgcgcctattacggcctactcccaacagacgcgaggcctacttggctgcatcatcaccagcctcacaggccgggacaggaaccaggtcgagggggaggtccaagtggtctccaccgcaacacaatctttcctggcgacctgcgtcaatggcgtgtgttggactgtctatcatggtgccggctcaaagacccttgccggcccaaagggcccaatcacccaaatgtacaccaatgtggaccaggacctcgtcggctggcaagcgccccccggggcgcgttccttgacaccatgcacctgcggcagctcggacctttacttggtcacgaagcatgccgatgtcattccggtgcgccggcggggcgacagcagggggagcctactctccccccggcccgtctcctacttgaagggctcttcgggcggtccactgctctgcccctcggggcacgctgtgggcatctttcgggctgccgtgtgcacccgaggggttgcgaaggcggtggtctttgtacccgtcgagtctatggaaaccactatgcggtccccggtcttcacggacaactcgtcccctccggccgtaccgcagacattccaggtggcccatctacacgcccctactggtagcggcaagagcactaaggtgccggctgcgtatgcagcccaagggtataaggtgcttgtcctgaacccgtccgtcgccgccaccctaggtttcggggcgtatatgtctaaggcacatggtatcgaccctaacatcagaaccggggtaaggaccatcaccacgggtgcccccatcacgtactccacctatggcaagtttcttgccgacggtggttgctctgggggcgcctatgacatcataatatgtgatgagtgccactcaactgactcgaccactatcctgggcatcggcacagtcctggaccaagcggagacggctggagcgcgactcgtcgtgctcgccaccgctacgcctccgggatcggtcaccgtgccacatccaaacatcgaggaggtggctctgtccagcactggagaaatccccttttatggcaaagccatccccatcgagaccatcaagggggggaggcacctcattttctgccattccaagaagaaatgcgatgagctcgccgcgaagctgtccggcctcggactcaatgctgtagcatattaccggggccttgatgtatccgtcataccaactagcggagacgtcattgtcgtagcaacggacgctctaatgacgggctttaccggcgatttcgactcagtgatcgactgcaatacatgtgtcacccagacagtcgacttcagcctggacccgaccttcaccattgagacgacgaccgtgccacaagacgcggtgtcacgctcgcagcggcgaggcaggactggtaggggcaggatgggcatttacaggtttgtgactccaggagaacggccctcgggcatgttcgattcctcggttctgtgcgagtgctatgacgcgggctgtgcttggtacgagctcacgcccgccgagacctcagttaggttgcgggcttacctaaacacaccagggttgcccgtctgccaggaccatctggagttctgggagggcgtctttacaggcctcacccacatagacgcccatttcttgtcccagactaagcaggcaggagacaacttcccctacctggtagcataccaggctacggtgtgcgccagggctcaggctccacctccatcgtgggaccaaatgtggaagtgtctcatacggctaaagcctacgctgcacgggccaacgcccctgctgtataggctgggagccgttcaaaacgaggttactaccacacaccccataaccaaatacatcatggcatgcatgtcggctgacctggaggtcgtcacgagcacctgggtgctggtaggcggagtcctagcagctctggctgcgtattgcctgacaacaggcagcgtggtcattgtgggcaggatcatcttgtccggaaggccggccatcattcccgacagggaagtcctttaccgggagttcgatgagatggaagagtgtgcctcacacctcccttacatcgaacagggaatgcagctcgccgaacaattcaaacagaaggcaatcgggttgctgcaaacagccaccaagcaagcggaggctgctgctcccgtggtggaatccaagtggcggaccctcgaagccttctgggcgaagcatatgtggaatttcatcagcgggatacaatatttagcaggcttgtccactctgcctggcaaccccgcgatagcatcactgatggcattcacagcctctatcaccagcccgctcaccacccaacataccctcctgtttaacatcctggggggatgggtggccgcccaacttgctcctcccagcgctgcttccgctttcgtaggcgccggcatcgctggagcggctgttggcagcataggccttgggaaggtgcttgtggatattttggcaggttatggagcaggggtggcaggcgcgctcgtggcctttaaggtcatgagcggcgagatgccctccaccgaggacctggttaacctactccctgctatcctctcccctggcgccctagtcgtcggggtcgtgtgcgcagcgatactgcgtcggcacgtgggcccaggggagggggctgtgcagtggatgaaccggctgatagcgttcgcttcgcggggtaaccacgtctcccccacgcactatgtgcctgagagcgacgctgcagcacgtgtcactcagatcctctctagtcttaccatcactcagctgctgaagaggcttcaccagtggatcaacgaggactgctccacgccatgctccggctcgtggctaagagatgtttgggattggatatgcacggtgttgactgatttcaaggcctggctccagtccaagctcctgccgcgattgccgggagtccccttcttctcatgtcaacgtgggtacaagggagtctggcggggcgacggcatcatgcaaaccacctgcccatgtggagcacagatcaccggacatgtgaaaaactgttccatgaggatcgtggggcctaggacctgtagtaacacgtggcatggaacattccccattaacgcgtacaccacgggcccctgcacgccctccccggcgccaaattattctagggcgctgtggcgggtggctgctgaggagtacgtggaggttacgcgagtgggggatttccactacgtgacgggcatgaccactgacaacgtaaagtgcccgtgtcaggttccggcccccgaattcttcacagaagtggatggggtgcggttgcacaggtacgctccagcgtgcaaacccctcctacgggaggaggtcacattcctggtcgggctcaatcaatacccggttgggtcacagctcccatgcgagcccgaactggacgtagcagtgctcacttccatgctcaccgacccctcccacattacggcggagacggctaagcgtaggctggccaggggatctcccccctccttggccagctcatcagctagccagctgtctgcgccttccttgaaggcaacatgcactacccgtcatgactccccggacgctgacctcatcgaggccaacctcctgtggcggcaggagatgggcgggaacatcacccgcgtggagtcagagaataaggtagtaattttggactctttcgagccgctccaagcggaggaggatgagagggaagtatccgttccggcggagatcctgcggaggtccaggaaattccctcgagcgatgcccatatgggcacgcccggattacaaccctccactgttagagtcctggaaggacccggactacgtccctccagtggtacacgggtgtccattgccgcctgccaaggcccctccgataccacctccacggaggaagaggacggttgtcctgtcagaatctaccgtgtcttctgccttggcggagctcgccacaaagaccttcggcagctccgaatcgtcggccgtcgacagcggcacggcaacggcctctcctgaccagccctccgacgacggcgacgcgggatccgacgttgagtcgtactcctccatgcccccccttgagggggagccgggggatcccgatctcagcgacgggtcttggtctaccgtaagcgaggagttaattaacgctagtgaggatatcgtctgctgctcgatgtcctacacatggacaggcgccctgatcacgccatgcgctgcggaggaaaccaagctgcccatcaatgcactgagcaactctttgctccgtcaccacaacttggtctatgctacaacatctcgcagcgcaagcctgcggcagaagaaggtcacctttgacagactgcaggtcctggacgaccactaccgggacgtgctcaaggagatgaaggcgaaggcgtccacagttaaggctaaacttctatccgtggaggaagcctgtaagctgacgcccccacattcggccagatctaaatttggctatggggcaaaggacgtccggaacctatccagcaaggccgttaaccacatccgctccgtgtggaaggacttgctggaagacactgagacaccaattgacaccaccatcatggcaaaaaatgaggttttctgcgtccaaccagagaaggggggccgcaagccagctcgccttatcgtattcccagatttgggggttcgtgtgtgcgagaaaatggccctttacgatgtggtctccaccctccctcaggccgtgatgggctcttcatacggattccaatactctcctggacagcgggtcgagttcctggtgaatgcctggaaagcgaagaaatgccctatgggcttcgcatatgacacccgctgttttgactcaacggtcactgagaatgacatccgtgttgaggagtcaatctaccaatgttgtgacttggcccccgaagccagacaggccataaggtcgctcacagagcggctttacatcgggggccccctgactaattctaaagggcagaactgcggctatcgccggtgccgcgcgagcggtgtactgacgaccagctgcggtaataccctcacatgttacttgaaggccgctgcggcctgtcgagctgcgaagctccaggactgcacgatgctcgtatgcggagacgaccttgtcgttatctgtgaaagcgcggggacccaagaggacgaggcgagcctacgggccttcacggaggctatgactagatactctgccccccctggggacccgcccaaaccagaatacgacttggagttgataacatcatgctcctccaatgtgtcagtcgcgcacgatgcatctggcaaaagggtgtactatctcacccgtgaccccaccaccccccttgcgcgggctgcgtgggagacagctagacacactccagtcaattcctggctaggcaacatcatcatgtatgcgcccaccttgtgggcaaggatgatcctgatgactcatttcttctccatccttctagctcaggaacaacttgaaaaagccctagattgtcagatctacggggcctgttactccattgagccacttgacctacctcagatcattcaacgactccacggccttagcgcattttcactccatagttactctccaggtgagatcaatagggtggcttcatgcctcaggaaacttggggtaccgcccttgcgagtctggagacatcgggccagaagtgtccgcgctaggctactgtcccagggggggagggctgccacttgtggcaagtacctcttcaactgggcagtaaggaccaagctcaaactcactccaatcccggctgcgtcccagttggatttatccagctggttcgttgctggttacagcgggggagacatatatcacagcctgtctcgtgcccgaccccgctggttcatgtggtgcctactcctactttctgtaggggtaggcatctatctactccccaaccgatga

SEQ ID NO: 4 - protein, 2204 aa, HCV
MDREMAASCGGAVFVGLILLTLSPHYKLFLARLIWWLQYFITRAEAHLQVWIPPLNVRGGRDAVILLTCAIHPELIFTITKILLAILGPLMVLQAGITKVPYFVRAHGLIRACMLVRKVAGGHYVQMALMKLAALTGTYVYDHLTPLRDWAHAGLRDLAVAVEPVVFSDMETKVITWGADTAACGDIILGLPVSARRGREIHLGPADSLEGQGWRLLAPITAYSQQTRGLLGCIITSLTGRDRNQVEGEVQVVSTATQSFLATCVNGVCWTVYHGAGSKTLAGPKGPITQMYTNVDQDLVGWQAPPGARSLTPCTCGSSDLYLVTKHADVIPVRRRGDSRGSLLSPRPVSYLKGSSGGPLLCPSGHAVGIFRAAVCTRGVAKAVVFVPVESMETTMRSPVFTDNSSPPAVPQTFQVAHLHAPTGSGKSTKVPAAYAAQGYKVLVLNPSVAATLGFGAYMSKAHGIDPNIRTGVRTITTGAPITYSTYGKFLADGGCSGGAYDIIICDECHSTDSTTILGIGTVLDQAETAGARLVVLATATPPGSVTVPHPNIEEVALSSTGEIPFYGKAIPIETIKGGRHLIFCHSKKKCDELAAKLSGLGLNAVAYYRGLDVSVIPTSGDVIVVATDALMTGFTGDFDSVIDCNTCVTQTVDFSLDPTFTIETTTVPQDAVSRSQRRGRTGRGRMGIYRFVTPGERPSGMFDSSVLCECYDAGCAWYELTPAETSVRLRAYLNTPGLPVCQDHLEFWEGVFTGLTHIDAHFLSQTKQAGDNFPYLVAYQATVCARAQAPPPSWDQMWKCLIRLKPTLHGPTPLLYRLGAVQNEVTTTHPITKYIMACMSADLEVVTSTWVLVGGVLAALAAYCLTTGSVVIVGRIILSGRPAIIPDREVLYREFDEMEECASHLPYIEQGMQLAEQFKQKAIGLLQTATKQAEAAAPVVESKWRTLEAFWAKHMWNFISGIQYLAGLSTLPGNPAIASLMAFTASITSPLTTQHTLLFNILGGWVAAQLAPPSAASAFVGAGIAGAAVGSIGLGKVLVDILAGYGAGVAGALVAFKVMSGEMPSTEDLVNLLPAILSPGALVVGVVCAAILRRHVGPGEGAVQWMNRLIAFASRGNHVSPTHYVPESDAAARVTQILSSLTITQLLKRLHQWINEDCSTPCSGSWLRDVWDWICTVLTDFKAWLQSKLLPRLPGVPFFSCQRGYKGVWRGDGIMQTTCPCGAQITGHVKNCSMRIVGPRTCSNTWHGTFPINAYTTGPCTPSPAPNYSRALWRVAAEEYVEVTRVGDFHYVTGMTTDNVKCPCQVPAPEFFTEVDGVRLHRYAPACKPLLREEVTFLVGLNQYPVGSQLPCEPELDVAVLTSMLTDPSHITAETAKRRLARGSPPSLASSSASQLSAPSLKATCTTRHDSPDADLIEANLLWRQEMGGNITRVESENKVVILDSFEPLQAEEDEREVSVPAEILRRSRKFPRAMPIWARPDYNPPLLESWKDPDYVPPVVHGCPLPPAKAPPIPPPRRKRTVVLSESTVSSALAELATKTFGSSESSAVDSGTATASPDQPSDDGDAGSDVESYSSMPPLEGEPGDPDLSDGSWSTVSEELINASEDIVCCSMSYTWTGALITPCAAEETKLPINALSNSLLRHHNLVYATTSRSASLRQKKVTFDRLQVLDDHYRDVLKEMKAKASTVKAKLLSVEEACKLTPPHSARSKFGYGAKDVRNLSSKAVNHIRSVWKDLLEDTETPIDTTIMAKNEVFCVQPEKGGRKPARLIVFPDLGVRVCEKMALYDVVSTLPQAVMGSSYGFQYSPGQRVEFLVNAWKAKKCPMGFAYDTRCFDSTVTENDIRVEESIYQCCDLAPEARQAIRSLTERLYIGGPLTNSKGQNCGYRRCRASGVLTTSCGNTLTCYLKAAAACRAAKLQDCTMLVCGDDLVVICESAGTQEDEASLRAFTEAMTRYSAPPGDPPKPEYDLELITSCSSNVSVAHDASGKRVYYLTRDPTTPLARAAWETARHTPVNSWLGNIIMYAPTLWARMILMTHFFSILLAQEQLEKALDCQIYGACYSIEPLDLPQIIQRLHGLSAFSLHSYSPGEINRVASCLRKLGVPPLRVWRHRARSVRARLLSQGGRAATCGKYLFNWAVRTKLKLTPIPAASQLDLSSWFVAGYSGGDIYHSLSRARPRWFMWCLLLLSVGVGIYLLPNR

SEQ ID NO: 5 - DNA, 6609 bp, HCV
  CDS (1)...(6609)
atggacacggaggtggccgcgtcgtgtggcggcgttgttcttgtcgggttaatggcgctgactctgtcgccatattacaagcgctatatcagctggtgcatgtggtggcttcagtattttctgaccagagtagaagcgcaactgcacgtgtgggttccccccctcaacgtccggggggggcgcgatgccgtcatcttactcatgtgtgtagtacacccgaccctggtatttgacatcaccaaactactcctggccatcttcggacccctttggattcttcaagccagtttgcttaaagtcccctacttcgtgcgcgttcaaggccttctccggatctgcgcgctagcgcggaagatagccggaggtcattacgtgcaaatggccatcatcaagttaggggcgcttactggcacctatgtgtataaccatctcacccctcttcgagactgggcgcacaacggcctgcgagatctggccgtggctgtggaaccagtcgtcttctcccgaatggagaccaagctcatcacgtggggggcagataccgccgcgtgcggtgacatcatcaacggcttgcccgtctctgcccgtaggggccaggagatactgcttgggccagccgacggaatggtctccaaggggtggaggttgctggcgcccatcacggcgtacgcccagcagacgagaggcctcctagggtgtataatcaccagcctgactggccgggacaaaaaccaagtggagggtgaggtccagatcgtgtcaactgctacccaaaccttcctggcaacgtgcatcaatggggtatgctggactgtctaccacggggccggaacgaggaccatcgcatcacccaagggtcctgtcatccagatgtataccaatgtggaccaagaccttgtgggctggcccgctcctcaaggttcccgctcattgacaccctgtacctgcggctcctcggacctttacctggtcacgaggcacgccgatgtcattcccgtgcgccggcgaggtgatagcaggggtagcctgctttcgccccggcccatttcctacttgaaaggctcctcggggggtccgctgttgtgccccgcgggacacgccgtgggcctattcagggccgcggtgtgcacccgtggagtggctaaagcggtggtctttatccctgtggagaacctagggactaccatgagatccccggtgttcacggacaactcctctccaccagcagtgccccagagcttccaggtggcccacctgcatgctcccaccggcagcggtaagagcaccaaggtcccggctgcgtacgcagcccagggctacaaggtgttggtgctcaacccctctgttgctgcaacgctgggctttggtgcttacatgtccaaggcccatggggttgatcctaatatcagggccggggtgagaacaattaccactggcagccccatcacgtactccacctacggcaagttccttgccgacggcgggtgctcagggggtgcttatgacataataatttgtgacgagtgccactccacggatgccacatccatcttgggcatcggcactgtccttgaccaagcagagactgcgggggcgagactggttgtgctcgccactgctacccctccgggctccgtcactgtgccccatcctaacatcgaggaggttgctctgtccaccaccggagagatccccttttacggcaaggctatccccctcgaggtgatcaaggggggaagacatctcatcttctgccactcaaagaagaagtgcgacgagctcgccgcgaagctggtcgcattgggcatcaatgccgtggcctactaccgcggtcttgacgtgtctgtaatcccgaccagcggcaatgttgtcgtcgtgtcgaccgatgctctcatgactggctttaccggcgacttcgactctgtgatagactgcaacacgtgtgtcactcagacagtcgatttcagccttgaccctacctttaccattgagacaaccacgctcccccaggatgctgtctccaggactcaacgccggggcaggactggcagggggaagccaggcatctatagatttgtggcaccgggggagcgcccctccggcatgttcgactcgtccgtcctctgtgagtgctatgacgcgggctgtgcttggtatgagctcacgcccgccgagactacagttaggctacgagcgtacatgaacaccccggggcttcccgtgtgccaggaccatcttgaattttgggagggcgtttttacgggcctcactcatatagatgcccactttttatcccagacaaagcagagtggggagaactttccttacctggtagcgtaccaagccaccgtgtgcgctagggctcaagcccctcccccatcgtgggaccagatgtggaagtgtttgatccgccttaaacccaccctccatgggccaacacccctgctatacagactgggcgctgttcagaatgaagtcaccctgacgcacccaatcaccaaatacatcatgacatgcatgtcggccgacctggaggtcgtcacgagcacctgggtgctcgttggcggcgtcctggctgctctggccgcgtattgcctgtcaacaggctgcgtggtcatagtgggcaggatcgtcttgtccgggaagccggcaattatacctgacagggaggttctctaccaggagttcgatgagatggaagagtgctctcagcacttaccgtacatcgagcaagggatgatgctcgctgagcagttcaagcagaaggccctcggcctcctgcagaccgcgtcccgccatgcagaggttatcacccctgctgtccagaccaactggcagaaactcgaggtcttttgggcgaagcacatgtggaatttcatcagtgggatacaatacttggcgggcctgtcaacgctgcctggtaaccccgccattgcttcattgatggcttttacagctgccgtcaccagcccactgaccactggccaaaccctcctcctcaacatattgggggggtgggtggctgcccagctcgccgcccccggtgccgctactgcctttgtgggtgctggcctagctggcgccgccgtcggcagcgttggactggggaaggtcctcgtggacattcttgcagggtatggcgcgggcgtggcgggagctcttgtagcattcaagatcatgagcggtgaggtcccctccacggaggacctggtcaatctgctgcccgccatcctctcgcctggagcccttgtagtcggtgtggtctgcgcagcaatactgcgccggcacgttggcccgggcgagggggcagtgcaatggatgaaccggctaatagccttcgcctcccgggggaaccatgtttcccccacgcactacgtgccggagagcgatgcagccgcccgcgtcactgccatactcagcagcctcactgtaacccagctcctgaggcgactgcatcagtggataagctcggagtgtaccactccatgctccggttcctggctaagggacatctgggactggatatgcgaggtgctgagcgactttaagacctggctgaaagccaagctcatgccacaactgcctgggattccctttgtgtcctgccagcgcgggtataggggggtctggcgaggagacggcattatgcacactcgctgccactgtggagctgagatcactggacatgtcaaaaacgggacgatgaggatcgtcggtcctaggacgtgcaggaacatgtggagtgggacgttccccattaacgcctacaccacgggcccctgtactccccttcctgcgccgaactataagttcgcgctgtggagggtgtctgcagaggaatacgtggagataaggcgggtgggggacttccactacgtatcgggtatgactactgacaatcttaaatgcccgtgccagatcccatcgcccgaatttttcacagaattggacggggtgcgcctacacaggtttgcgcccccttgcaagcccttgctgcgggaggaggtatcattcagagtaggactccacgagtacccggtggggtcgcaattaccttgcgagcccgaaccggacgtagccgtgttgacgtccatgctcactgatccctcccatataacagcagaggcggccgggagaaggttggcgagagggtcacccccttctatggccagctcctcggctagccagctgtccgctccatctctcaaggcaacttgcaccgccaaccatgactcccctgacgccgagctcatagaggctaacctcctgtggaggcaggagatgggcggcaacatcaccagggttgagtcagagaacaaagtggtgattctggactccttcgatccgcttgtggcagaggaggatgagcgggaggtctccgtacctgcagaaattctgcggaagtctcggagattcgcccgggccctgcccgtctgggcgcggccggactacaaccccccgctagtagagacgtggaaaaagcctgactacgaaccacctgtggtccatggctgcccgctaccacctccacggtcccctcctgtgcctccgcctcggaaaaagcgtacggtggtcctcaccgaatcaaccctatctactgccttggccgagcttgccaccaaaagttttggcagctcctcaacttccggcattacgggcgacaatacgacaacatcctctgagcccgccccttctggctgcccccccgactccgacgttgagtcctattcttccatgccccccctggagggggagcctggggatccggatctcagcgacgggtcatggtcgacggtcagtagtggggccgacacggaagatgtcgtgtgctgctcaatgtcttattcctggacaggcgcactcgtcaccccgtgcgctgcggaagaacaaaaactgcccatcaacgcactgagcaactcgttgctacgccatcacaatctggtgtattccaccacttcacgcagtgcttgccaaaggcagaagaaagtcacatttgacagactgcaagttctggacagccattaccaggacgtgctcaaggaggtcaaagcagcggcgtcaaaagtgaaggctaacttgctatccgtagaggaagcttgcagcctgacgcccccacattcagccaaatccaagtttggctatggggcaaaagacgtccgttgccatgccagaaaggccgtagcccacatcaactccgtgtggaaagaccttctggaagacagtgtaacaccaatagacactaccatcatggccaagaacgaggttttctgcgttcagcctgagaaggggggtcgtaagccagctcgtctcatcgtgttccccgacctgggcgtgcgcgtgtgcgagaagatggccctgtacgacgtggttagcaagctccccctggccgtgatgggaagctcctacggattccaatactcaccaggacagcgggttgaattcctcgtgcaagcgtggaagtccaagaagaccccgatggggttctcgtatgatacccgctgttttgactccacagtcactgagagcgacatccgtacggaggaggcaatttaccaatgttgtgacctggacccccaagcccgcgtggccatcaagtccctcactgagaggctttatgttgggggccctcttaccaattcaaggggggaaaactgcggctaccgcaggtgccgcgcgagcggcgtactgacaactagctgtggtaacaccctcacttgctacatcaaggcccgggcagcctgtcgagccgcagggctccaggactgcaccatgctcgtgtgtggcgacgacttagtcgttatctgtgaaagtgcgggggtccaggaggacgcggcgagcctgagagccttcacggaggctatgaccaggtactccgccccccccggggaccccccacaaccagaatacgacttggagcttataacatcatgctcctccaacgtgtcagtcgcccacgacggcgctggaaagagggtctactaccttacccgtgaccctacaacccccctcgcgagagccgcgtgggagacagcaagacacactccagtcaattcctggctaggcaacataatcatgtttgcccccacactgtgggcgaggatgatactgatgacccatttctttagcgtcctcatagccagggatcagcttgaacaggctcttaactgtgagatctacggagcctgctactccatagaaccactggatctacctccaatcattcaaagactccatggcctcagcgcattttcactccacagttactctccaggtgaaatcaatagggtggccgcatgcctcagaaaacttggggtcccgcccttgcgagcttggagacaccgggcccggagcgtccgcgctaggcttctgtccagaggaggcagggctgccatatgtggcaagtacctcttcaactgggcagtaagaacaaagctcaaactcactccaatagcggccgctggccggctggacttgtccggttggttcacggctggctacagcgggggagacatttatcacagcgtgtctcatgcccggccccgctggttctggttttgcctactcctgctcgctgcaggggtaggcatctacctcctccccaaccgatga

SEQ ID NO: 6 - protein, 2202 aa, HCV
MDTEVAASCGGVVLVGLMALTLSPYYKRYISWCMWWLQYFLTRVEAQLHVWVPPLNVRGGRDAVILLMCVVHPTLVFDITKLLLAIFGPLWILQASLLKVPYFVRVQGLLRICALARKIAGGHYVQMAIIKLGALTGTYVYNHLTPLRDWAHNGLRDLAVAVEPVVFSRMETKLITWGADTAACGDIINGLPVSARRGQEILLGPADGMVSKGWRLLAPITAYAQQTRGLLGCIITSLTGRDKNQVEGEVQIVSTATQTFLATCINGVCWTVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVVFIPVENLGTTMRSPVFTDNSSPPAVPQSFQVAHLHAPTGSGKSTKVPAAYAAQGYKVLVLNPSVAATLGFGAYMSKAHGVDPNIRAGVRTITTGSPITYSTYGKFLADGGCSGGAYDIIICDECHSTDATSILGIGTVLDQAETAGARLVVLATATPPGSVTVPHPNIEEVALSTTGEIPFYGKAIPLEVIKGGRHLIFCHSKKKCDELAAKLVALGINAVAYYRGLDVSVIPTSGNVVVVSTDALMTGFTGDFDSVIDCNTCVTQTVDFSLDPTFTIETTTLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENFPYLVAYQATVCARAQAPPPSWDQMWKCLIRLKPTLHGPTPLLYRLGAVQNEVTLTHPITKYIMTCMSADLEVVTSTWVLVGGVLAALAAYCLSTGCVVIVGRIVLSGKPAIIPDREVLYQEFDEMEECSQHLPYIEQGMMLAEQFKQKALGLLQTASRHAEVITPAVQTNWQKLEVFWAKHMWNFISGIQYLAGLSTLPGNPAIASLMAFTAAVTSPLTTGQTLLLNILGGWVAAQLAAPGAATAFVGAGLAGAAVGSVGLGKVLVDILAGYGAGVAGALVAFKIMSGEVPSTEDLVNLLPAILSPGALVVGVVCAAILRRHVGPGEGAVQWMNRLIAFASRGNHVSPTHYVPESDAAARVTAILSSLTVTQLLRRLHQWISSECTTPCSGSWLRDIWDWICEVLSDFKTWLKAKLMPQLPGIPFVSCQRGYRGVWRGDGIMHTRCHCGAEITGHVKNGTMRIVGPRTCRNMWSGTFPINAYTTGPCTPLPAPNYKFALWRVSAEEYVEIRRVGDFHYVSGMTTDNLKCPCQIPSPEFFTELDGVRLHRFAPPCKPLLREEVSFRVGLHEYPVGSQLPCEPEPDVAVLTSMLTDPSHITAEAAGRRLARGSPPSMASSSASQLSAPSLKATCTANHDSPDAELIEANLLWRQEMGGNITRVESENKVVILDSFDPLVAEEDEREVSVPAEILRKSRRFARALPVWARPDYNPPLVETWKKPDYEPPVVHGCPLPPPRSPPVPPPRKKRTVVLTESTLSTALAELATKSFGSSSTSGITGDNTTTSSEPAPSGCPPDSDVESYSSMPPLEGEPGDPDLSDGSWSTVSSGADTEDVVCCSMSYSWTGALVTPCAAEEQKLPINALSNSLLRHHNLVYSTTSRSACQRQKKVTFDRLQVLDSHYQDVLKEVKAAASKVKANLLSVEEACSLTPPHSAKSKFGYGAKDVRCHARKAVAHINSVWKDLLEDSVTPIDTTIMAKNEVFCVQPEKGGRKPARLIVFPDLGVRVCEKMALYDVVSKLPLAVMGSSYGFQYSPGQRVEFLVQAWKSKKTPMGFSYDTRCFDSTVTESDIRTEEAIYQCCDLDPQARVAIKSLTERLYVGGPLTNSRGENCGYRRCRASGVLTTSCGNTLTCYIKARAACRAAGLQDCTMLVCGDDLVVICESAGVQEDAASLRAFTEAMTRYSAPPGDPPQPEYDLELITSCSSNVSVAHDGAGKRVYYLTRDPTTPLARAAWETARHTPVNSWLGNIIMFAPTLWARMILMTHFFSVLIARDQLEQALNCEIYGACYSIEPLDLPPIIQRLHGLSAFSLHSYSPGEINRVAACLRKLGVPPLRAWRHRARSVRARLLSRGGRAAICGKYLFNWAVRTKLKLTPIAAAGRLDLSGWFTAGYSGGDIYHSVSHARPRWFWFCLLLLAAGVGIYLLPNR